SEQUENCE LISTING (114 sequences)

SEQ ID NO: 1 - protein, 93 aa, Homo sapiens
MIHLGHILFLLLLPVAAAQTTPGERSSLPAFYPGTSGSCSGCGSLSLPLLAGLVAADAVASLLIVGAVFLCARPRRSPAQEDGKVYINMPGRG

SEQ ID NO: 2 - protein, 75 aa, Homo sapiens
QTTPGERSSLPAFYPGTSGSCSGCGSLSLPLLAGLVAADAVASLLIVGAVFLCARPRRSPAQEDGKVYINMPGRG

SEQ ID NO: 3 - protein, 51 aa, Homo sapiens
QTTPGERSSLPAFYPGTSGSCSGCGSLSLPLLAGLVAADAVASLLIVGAVF

SEQ ID NO: 4 - protein, 71 aa, Homo sapiens
MIHLGHILFLLLLPVAAAQTTPGERSSLPAFYPGTSGSCSGCGSLSLPLLAGLVAADAVASLLIVGAVFLC

SEQ ID NO: 5 - protein, 53 aa, Homo sapiens
QTTPGERSSLPAFYPGTSGSCSGCGSLSLPLLAGLVAADAVASLLIVGAVFLC

SEQ ID NO: 6 - protein, 24 aa, Homo sapiens
LCARPRRSPAQEDGKVYINMPGRG

SEQ ID NO: 7 - protein, 45 aa, Homo sapiens
LLAGLVAADAVASLLIVGAVFLCARPRRSPAQEDGKVYINMPGRG

SEQ ID NO: 8 - protein, 21 aa, Homo sapiens
LLAGLVAADAVASLLIVGAVF

SEQ ID NO: 9 - protein, 113 aa, Homo sapiens
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 10 - protein, 92 aa, Homo sapiens
LRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 11 - protein, 52 aa, Homo sapiens
YFLGRLVPRGRGAAEAATRKQRITETESPYQELQGQRSDVYSDLNTQRPYYK

SEQ ID NO: 12 - protein, 21 aa, Homo sapiens
GVLAGIVMGDLVLTVLIALAV

SEQ ID NO: 13 - protein, 40 aa, Homo sapiens
LRPVQAQAQSDCSCSTVSPGVLAGIVMGDLVLTVLIALAV

SEQ ID NO: 14 - protein, 216 aa, Homo sapiens
MGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 15 - protein, 144 aa, Homo sapiens
IWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 16 - protein, 135 aa, Homo sapiens
LFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 17 - protein, 165 aa, Homo sapiens
PFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 18 - skipped

SEQ ID NO: 19 - protein, 5 aa, synthetic construct
GSGGG

SEQ ID NO: 20 - protein, 4 aa, synthetic construct
GSGG

SEQ ID NO: 21 - protein, 4 aa, synthetic construct
SGGG

SEQ ID NO: 22 - protein, 5 aa, synthetic construct
GGGGS

SEQ ID NO: 23 - protein, 20 aa, synthetic construct
GGGGSGGGGSGGGGSGGGGS

SEQ ID NO: 24 - protein, 15 aa, synthetic construct
GGGGSGGGGSGGGGS

SEQ ID NO: 25 - protein, 5 aa, synthetic construct
GPPGS

SEQ ID NO: 26 - protein, 4 aa, synthetic construct
GGGS

SEQ ID NO: 27 - protein, 5 aa, synthetic construct
GGGGS

SEQ ID NO: 28-29 - skipped (2 entries)

SEQ ID NO: 30 - protein, 5 aa, synthetic construct
SGGGG

SEQ ID NO: 31 - protein, 4 aa, synthetic construct
SGGG

SEQ ID NO: 32 - skipped

SEQ ID NO: 33 - protein, 4 aa, synthetic construct
SGSG

SEQ ID NO: 34 - skipped

SEQ ID NO: 35 - protein, 5 aa, synthetic construct
GGGGA

SEQ ID NO: 36 - protein, 4 aa, synthetic construct
GGGA

SEQ ID NO: 37 - protein, 5 aa, synthetic construct
EAAAK

SEQ ID NO: 38 - protein, 4 aa, synthetic construct
RRKR

SEQ ID NO: 39 - protein, 19 aa, synthetic construct
ATNFSLLKQAGDVEENPGP

SEQ ID NO: 40 - protein, 18 aa, synthetic construct
EGRGSLLTCGDVEENPGP

SEQ ID NO: 41 - protein, 23 aa, synthetic construct
SGSGATNFSLLKQAGDVEENPGP

SEQ ID NO: 42 - protein, 22 aa, synthetic construct
SGSGEGRGSLLTCGDVEENPGP

SEQ ID NO: 43 - protein, 27 aa, synthetic construct
RRKRSGSGATNFSLLKQAGDVEENPGP

SEQ ID NO: 44 - protein, 26 aa, synthetic construct
RRKRSGSGEGRGSLLTCGDVEENPGP

SEQ ID NO: 45 - protein, 22 aa, synthetic construct
VKQTLNFDLLKLAGDVESNPGP

SEQ ID NO: 46 - protein, 20 aa, synthetic construct
QCTNYALLKLAGDVESNPGP

SEQ ID NO: 47 - protein, 6 aa, synthetic construct
HHHHHH

SEQ ID NO: 48 - protein, 8 aa, synthetic construct
DYKDDDDK

SEQ ID NO: 49 - protein, 15 aa, synthetic construct
GLNDIFEAQKIEWHE

SEQ ID NO: 50 - protein, 14 aa, synthetic construct
GKPIPNPLLGLDST

SEQ ID NO: 51 - protein, 9 aa, synthetic construct
IPNPLLGLD

SEQ ID NO: 52 - protein, 10 aa, synthetic construct
EQKLISEEDL

SEQ ID NO: 53 - protein, 31 aa, synthetic construct
GLNDIFEAQKIEWHEGGHHHHHHDYKDDDDK

SEQ ID NO: 54 - protein, 31 aa, synthetic construct
DYKDDDDKHHHHHHGGGLNDIFEAQKIEWHE

SEQ ID NO: 55 - protein, 21 aa, synthetic construct
MALPVTALLLPLALLLHAARP

SEQ ID NO: 56 - protein, 42 aa, synthetic construct
KRGRKKLLYIFKQPFMRPVQTTQEEDGCSCRFPEEEEGGCEL

SEQ ID NO: 57 - protein, 48 aa, synthetic construct
QRRKYRSNKGESPVEPAEPCHYSCPREEEGSTIPIQEDYRKPEPACSP

SEQ ID NO: 58 - protein, 12 aa, Homo sapiens
EPKSCDKTHTCP

SEQ ID NO: 59 - protein, 45 aa, Homo sapiens
TTTPAPRPPTPAPTIASQPLSLRPEACRPAAGGAVHTRGLDFACD

SEQ ID NO: 60 - protein, 145 aa, synthetic construct
MIHLGHILFLLLLPVAAAQTTPGERSSLPAFYPGTSGSCSGCGSLSLPLLAGLVAADAVASLLIVGAVFLCARPRRSPAQEDGKVYINMPGRGYFLGRLVPRGRGAAEAATRKQRITETESPYQELQGQRSDVYSDLNTQRPYYK

SEQ ID NO: 61 - protein, 132 aa, synthetic construct
MALPVTALLLPLALLLHAARPDYKDDDDKQTTPGERSSLPAFYPGTSGSCSGCGSLSLPLLAGLVAADAVASLLIVGAVFYFLGRLVPRGRGAAEAATRKQRITETESPYQELQGQRSDVYSDLNTQRPYYK

SEQ ID NO: 62 - protein, 138 aa, synthetic construct
MALPVTALLLPLALLLHAARPDYKDDDDKEPKSCDKTHTCPLLAGLVAADAVASLLIVGAVFLCARPRRSPAQEDGKVYINMPGRGYFLGRLVPRGRGAAEAATRKQRITETESPYQELQGQRSDVYSDLNTQRPYYK

SEQ ID NO: 63 - protein, 171 aa, synthetic construct
MALPVTALLLPLALLLHAARPDYKDDDDKTTTPAPRPPTPAPTIASQPLSLRPEACRPAAGGAVHTRGLDFACDLLAGLVAADAVASLLIVGAVFLCARPRRSPAQEDGKVYINMPGRGYFLGRLVPRGRGAAEAATRKQRITETESPYQELQGQRSDVYSDLNTQRPYYK

SEQ ID NO: 64 - protein, 388 aa, synthetic construct
MIHLGHILFLLLLPVAAAQTTPGERSSLPAFYPGTSGSCSGCGSLSLPLLAGLVAADAVASLLIVGAVFLCARPRRSPAQEDGKVYINMPGRGYFLGRLVPRGRGAAEAATRKQRITETESPYQELQGQRSDVYSDLNTQRPYYKRRKRSGSGATNFSLLKQAGDVEENPGPMGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 65 - protein, 494 aa, synthetic construct
MALPVTALLLPLALLLHAARPDYKDDDDKQTTPGERSSLPAFYPGTSGSCSGCGSLSLPLLAGLVAADAVASLLIVGAVFYFLGRLVPRGRGAAEAATRKQRITETESPYQELQGQRSDVYSDLNTQRPYYKRRKRSGSGEGRGSLLTCGDVEENPGPMIHLGHILFLLLLPVAAAQTTPGERSSLPAFYPGTSGSCSGCGSLSLPLLAGLVAADAVASLLIVGAVFLCARPRRSPAQEDGKVYINMPGRGRRKRSGSGATNFSLLKQAGDVEENPGPMGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 66 - protein, 527 aa, synthetic construct
MIHLGHILFLLLLPVAAAQTTPGERSSLPAFYPGTSGSCSGCGSLSLPLLAGLVAADAVASLLIVGAVFLCARPRRSPAQEDGKVYINMPGRGYFLGRLVPRGRGAAEAATRKQRITETESPYQELQGQRSDVYSDLNTQRPYYKRRKRSGSGATNFSLLKQAGDVEENPGPMGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVRRKRSGSGEGRGSLLTCGDVEENPGPMIHLGHILFLLLLPVAAAQTTPGERSSLPAFYPGTSGSCSGCGSLSLPLLAGLVAADAVASLLIVGAVFLCKRGRKKLLYIFKQPFMRPVQTTQEEDGCSCRFPEEEEGGCEL

SEQ ID NO: 67 - protein, 538 aa, synthetic construct
MIHLGHILFLLLLPVAAAQTTPGERSSLPAFYPGTSGSCSGCGSLSLPLLAGLVAADAVASLLIVGAVFLCARPRRSPAQEDGKVYINMPGRGYFLGRLVPRGRGAAEAATRKQRITETESPYQELQGQRSDVYSDLNTQRPYYKRRKRSGSGATNFSLLKQAGDVEENPGPMGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVRRKRSGSGEGRGSLLTCGDVEENPGPMALPVTALLLPLALLLHAARPDYKDDDDKQTTPGERSSLPAFYPGTSGSCSGCGSLSLPLLAGLVAADAVASLLIVGAVFLCKRGRKKLLYIFKQPFMRPVQTTQEEDGCSCRFPEEEEGGCEL

SEQ ID NO: 68 - protein, 533 aa, synthetic construct
MIHLGHILFLLLLPVAAAQTTPGERSSLPAFYPGTSGSCSGCGSLSLPLLAGLVAADAVASLLIVGAVFLCARPRRSPAQEDGKVYINMPGRGYFLGRLVPRGRGAAEAATRKQRITETESPYQELQGQRSDVYSDLNTQRPYYKRRKRSGSGATNFSLLKQAGDVEENPGPMGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVRRKRSGSGEGRGSLLTCGDVEENPGPMIHLGHILFLLLLPVAAAQTTPGERSSLPAFYPGTSGSCSGCGSLSLPLLAGLVAADAVASLLIVGAVFLCQRRKYRSNKGESPVEPAEPCHYSCPREEEGSTIPIQEDYRKPEPACSP

SEQ ID NO: 69 - protein, 544 aa, synthetic construct
MIHLGHILFLLLLPVAAAQTTPGERSSLPAFYPGTSGSCSGCGSLSLPLLAGLVAADAVASLLIVGAVFLCARPRRSPAQEDGKVYINMPGRGYFLGRLVPRGRGAAEAATRKQRITETESPYQELQGQRSDVYSDLNTQRPYYKRRKRSGSGATNFSLLKQAGDVEENPGPMGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVRRKRSGSGEGRGSLLTCGDVEENPGPMALPVTALLLPLALLLHAARPDYKDDDDKQTTPGERSSLPAFYPGTSGSCSGCGSLSLPLLAGLVAADAVASLLIVGAVFLCQRRKYRSNKGESPVEPAEPCHYSCPREEEGSTIPIQEDYRKPEPACSP

SEQ ID NO: 70 - DNA, 435 bp, synthetic construct
  source 1..435: mol_type = other DNA
atgatccacctgggccacatcctgttcctgctgctgctgcccgtggccgctgcccagaccacccctggcgagcggagcagcctgcctgccttctaccctggcaccagcggcagctgcagcggctgcggcagcctgagcctgcccctgctggccggcctggtggccgccgacgccgtggccagcctgctgatcgtgggcgccgtgttcctgtgcgccaggcccaggcggagccctgcccaggaggacggcaaggtgtacatcaacatgcccggccggggctacttcctgggcaggctggtgcccaggggcaggggcgctgccgaggctgccacccggaagcagcggatcaccgagaccgagagcccctaccaggagctgcagggccagcggagcgacgtgtacagcgacctgaacacccagaggccctactacaag

SEQ ID NO: 71 - DNA, 396 bp, synthetic construct
  source 1..396: mol_type = other DNA
atggctctgcctgtgacagctctgctgctgcctctggctctgctgctgcacgccgctagacccgattataaggacgacgacgacaagcagaccacccctggcgagcggagcagcctgcctgccttctaccctggcaccagcggcagctgcagcggctgcggcagcctgagcctgcccctgctggctggcctggtggccgccgacgccgtggccagcctgctgatcgtgggcgccgtgttctacttcctgggcaggctggtgcccaggggcaggggcgctgccgaggctgccacccggaagcagcggatcaccgagaccgagagcccctaccaggagctgcagggccagcggagcgacgtgtacagcgacctgaacacccagaggccctactacaag

SEQ ID NO: 72 - DNA, 414 bp, synthetic construct
  source 1..414: mol_type = other DNA
atggctctgcctgtgacagctctgctgctgcctctggctctgctgctgcacgccgctagacccgattataaggacgacgacgacaaggagcccaagagctgcgacaagacacacacatgccctcttctggccggcctggtggccgccgacgccgtggccagcctgctgatcgtgggcgccgtgttcctgtgcgccaggcccaggcggagccctgcccaggaggacggcaaggtgtacatcaacatgcccggccggggctacttcctgggcaggctggtgcccaggggcaggggcgctgccgaggctgccacccggaagcagcggatcaccgagaccgagagcccctaccaggagctgcagggccagcggagcgacgtgtacagcgacctgaacacccagaggccctactacaag

SEQ ID NO: 73 - DNA, 513 bp, synthetic construct
  source 1..513: mol_type = other DNA
atggctctgcctgtgacagctctgctgctgcctctggctctgctgctgcacgccgctagacccgattataaggacgacgacgacaagaccacaacacctgctcctagacctcccacccctgctcccaccatcgccagccagcccctgagcctgagacccgaggcctgcagacccgctgctggcggcgctgtgcataccagaggcctggatttcgcctgcgaccttctggccggcctggtggccgccgacgccgtggccagcctgctgatcgtgggcgccgtgttcctgtgcgccaggcccaggcggagccctgcccaggaggacggcaaggtgtacatcaacatgcccggccggggctacttcctgggcaggctggtgcccaggggcaggggcgctgccgaggctgccacccggaagcagcggatcaccgagaccgagagcccctaccaggagctgcagggccagcggagcgacgtgtacagcgacctgaacacccagaggccctactacaag

SEQ ID NO: 74 - DNA, 1164 bp, synthetic construct
  source 1..1164: mol_type = other DNA
atgatccacctgggccacatcctgttcctgctgctgctgcccgtggccgctgcccagaccacccctggcgagcggagcagcctgcctgccttctaccctggcaccagcggcagctgcagcggctgcggcagcctgagcctgcccctgctggccggcctggtggccgccgacgccgtggccagcctgctgatcgtgggcgccgtgttcctgtgcgccaggcccaggcggagccctgcccaggaggacggcaaggtgtacatcaacatgcccggccggggctacttcctgggcaggctggtgcccaggggcaggggcgctgccgaggctgccacccggaagcagcggatcaccgagaccgagagcccctaccaggagctgcagggccagcggagcgacgtgtacagcgacctgaacacccagaggccctactacaagaggcggaaaaggtctgggagtggggctaccaatttctctctcctcaagcaagccggagacgttgaggaaaaccctggacccatgggctggatccggggacggaggagccggcacagctgggagatgagcgagttccacaactacaacctggacctgaagaagagcgacttcagcacccggtggcagaagcagcggtgccccgtggtgaagagcaagtgccgggagaacgccagccccttcttcttctgctgcttcatcgccgtggctatgggcatccggttcatcatcatggtggccatctggagcgccgtgttcctgaacagcctgttcaaccaggaggtgcagatccccctgaccgagagctactgcggcccctgccccaagaactggatctgctacaagaacaactgctaccagttcttcgacgagagcaagaactggtacgagagccaggccagctgcatgagccagaacgccagcctgctgaaggtgtacagcaaggaggaccaggacctgctgaagctggtgaagagctaccactggatgggcctggtgcacatccccaccaacggcagctggcagtgggaggacggcagcatcctgagccccaacctgctgaccatcatcgagatgcagaagggcgactgcgccctgtacgccagcagcttcaagggctacatcgagaactgcagcacccccaacacctacatctgcatgcagcggaccgtg

SEQ ID NO: 75 - DNA, 1482 bp, synthetic construct
  source 1..1482: mol_type = other DNA
atggctctgcctgtgacagctctgctgctgcctctggctctgctgctgcacgccgctagacccgattataaggacgacgacgacaagcagaccacccctggcgagcggagcagcctgcctgccttctaccctggcaccagcggcagctgcagcggctgcggcagcctgagcctgcccctgctggctggcctggtggccgccgacgccgtggccagcctgctgatcgtgggcgccgtgttctacttcctgggcaggctggtgcccaggggcaggggcgctgccgaggctgccacccggaagcagcggatcaccgagaccgagagcccctaccaggagctgcagggccagcggagcgacgtgtacagcgacctgaacacccagaggccctactacaagcggagaaagcgctccggctccggcgagggccgcggcagcctgctgacctgcggcgacgtggaagagaaccccggacccatgatccacctgggccacatcctgttcctgctgctgctgcccgtggccgctgcccaaacaacacccggcgagagatcctccttgcccgctttctatcccggaacatccggaagctgttccggatgtggatccctttctttgcctttgcttgctggattggtcgcagctgacgctgtcgcttccctccttattgtcggagctgtcttcctgtgcgccaggcccaggcggagccctgcccaggaggacggcaaggtgtacatcaacatgcccggccggggcaggcggaagcgctccgggagtggggctaccaatttctctctcctcaagcaagccggagacgttgaggaaaaccctggacccatgggctggatccggggacggaggagccggcacagctgggagatgagcgagttccacaactacaacctggacctgaagaagagcgacttcagcacccggtggcagaagcagcggtgccccgtggtgaagagcaagtgccgggagaacgccagccccttcttcttctgctgcttcatcgccgtggctatgggcatccggttcatcatcatggtggccatctggagcgccgtgttcctgaacagcctgttcaaccaggaggtgcagatccccctgaccgagagctactgcggcccctgccccaagaactggatctgctacaagaacaactgctaccagttcttcgacgagagcaagaactggtacgagagccaggccagctgcatgagccagaacgccagcctgctgaaggtgtacagcaaggaggaccaggacctgctgaagctggtgaagagctaccactggatgggcctggtgcacatccccaccaacggcagctggcagtgggaggacggcagcatcctgagccccaacctgctgaccatcatcgagatgcagaagggcgactgcgccctgtacgccagcagcttcaagggctacatcgagaactgcagcacccccaacacctacatctgcatgcagcggaccgtg

SEQ ID NO: 76 - DNA, 1581 bp, synthetic construct
  source 1..1581: mol_type = other DNA
atgatccacctgggccacatcctgttcctgctgctgctgcccgtggccgctgcccagaccacccctggcgagcggagcagcctgcctgccttctaccctggcaccagcggcagctgcagcggctgcggcagcctgagcctgcccctgctggccggcctggtggccgccgacgccgtggccagcctgctgatcgtgggcgccgtgttcctgtgcgccaggcccaggcggagccctgcccaggaggacggcaaggtgtacatcaacatgcccggccggggctacttcctgggcaggctggtgcccaggggcaggggcgctgccgaggctgccacccggaagcagcggatcaccgagaccgagagcccctaccaggagctgcagggccagcggagcgacgtgtacagcgacctgaacacccagaggccctactacaagaggcggaaaaggtctgggagtggggctaccaatttctctctcctcaagcaagccggagacgttgaggaaaaccctggacccatgggctggatccggggacggaggagccggcacagctgggagatgagcgagttccacaactacaacctggacctgaagaagagcgacttcagcacccggtggcagaagcagcggtgccccgtggtgaagagcaagtgccgggagaacgccagccccttcttcttctgctgcttcatcgccgtggctatgggcatccggttcatcatcatggtggccatctggagcgccgtgttcctgaacagcctgttcaaccaggaggtgcagatccccctgaccgagagctactgcggcccctgccccaagaactggatctgctacaagaacaactgctaccagttcttcgacgagagcaagaactggtacgagagccaggccagctgcatgagccagaacgccagcctgctgaaggtgtacagcaaggaggaccaggacctgctgaagctggtgaagagctaccactggatgggcctggtgcacatccccaccaacggcagctggcagtgggaggacggcagcatcctgagccccaacctgctgaccatcatcgagatgcagaagggcgactgcgccctgtacgccagcagcttcaagggctacatcgagaactgcagcacccccaacacctacatctgcatgcagcggaccgtgagaagaaagagaagcggcagcggcgagggcagaggcagcctgctgacctgcggcgacgtggaggagaaccccggacccatgattcatctcggacatattctctttctcttgctcttgcctgtcgctgccgctcaaacaactcccggagaaagatcttctctccccgctttttatcccggaacatctggatcttgttctggatgtggatctttgtctctccctctcctcgctggactcgtcgcagctgatgctgtcgcttctctcttgattgtcggagctgtctttttgtgtaagagaggcagaaagaagctgctgtacatcttcaagcagcccttcatgagacccgtgcagaccacccaggaggaggacggctgcagctgcagattccccgaggaggaggagggcggctgcgagctg

SEQ ID NO: 77 - DNA, 1614 bp, synthetic construct
  source 1..1614: mol_type = other DNA
atgatccacctgggccacatcctgttcctgctgctgctgcccgtggccgctgcccagaccacccctggcgagcggagcagcctgcctgccttctaccctggcaccagcggcagctgcagcggctgcggcagcctgagcctgcccctgctggccggcctggtggccgccgacgccgtggccagcctgctgatcgtgggcgccgtgttcctgtgcgccaggcccaggcggagccctgcccaggaggacggcaaggtgtacatcaacatgcccggccggggctacttcctgggcaggctggtgcccaggggcaggggcgctgccgaggctgccacccggaagcagcggatcaccgagaccgagagcccctaccaggagctgcagggccagcggagcgacgtgtacagcgacctgaacacccagaggccctactacaagaggcggaaaaggtctgggagtggggctaccaatttctctctcctcaagcaagccggagacgttgaggaaaaccctggacccatgggctggatccggggacggaggagccggcacagctgggagatgagcgagttccacaactacaacctggacctgaagaagagcgacttcagcacccggtggcagaagcagcggtgccccgtggtgaagagcaagtgccgggagaacgccagccccttcttcttctgctgcttcatcgccgtggctatgggcatccggttcatcatcatggtggccatctggagcgccgtgttcctgaacagcctgttcaaccaggaggtgcagatccccctgaccgagagctactgcggcccctgccccaagaactggatctgctacaagaacaactgctaccagttcttcgacgagagcaagaactggtacgagagccaggccagctgcatgagccagaacgccagcctgctgaaggtgtacagcaaggaggaccaggacctgctgaagctggtgaagagctaccactggatgggcctggtgcacatccccaccaacggcagctggcagtgggaggacggcagcatcctgagccccaacctgctgaccatcatcgagatgcagaagggcgactgcgccctgtacgccagcagcttcaagggctacatcgagaactgcagcacccccaacacctacatctgcatgcagcggaccgtgagaagaaagagaagcggcagcggcgagggcagaggcagcctgctgacctgcggcgacgtggaggagaaccccggacctatggctctgcctgtgacagctctgctgctgcctctggctctgctgctgcacgccgctagacccgattataaggacgacgacgacaagcaaacaactcccggagaaagatcttctctccccgctttttatcccggaacatctggatcttgttctggatgtggatctttgtctctccctctcctcgctggactcgtcgcagctgatgctgtcgcttctctcttgattgtcggagctgtctttttgtgtaagagaggcagaaagaagctgctgtacatcttcaagcagcccttcatgagacccgtgcagaccacccaggaggaggacggctgcagctgcagattccccgaggaggaggagggcggctgcgagctg

SEQ ID NO: 78 - DNA, 1599 bp, synthetic construct
  source 1..1599: mol_type = other DNA
atgatccacctgggccacatcctgttcctgctgctgctgcccgtggccgctgcccagaccacccctggcgagcggagcagcctgcctgccttctaccctggcaccagcggcagctgcagcggctgcggcagcctgagcctgcccctgctggccggcctggtggccgccgacgccgtggccagcctgctgatcgtgggcgccgtgttcctgtgcgccaggcccaggcggagccctgcccaggaggacggcaaggtgtacatcaacatgcccggccggggctacttcctgggcaggctggtgcccaggggcaggggcgctgccgaggctgccacccggaagcagcggatcaccgagaccgagagcccctaccaggagctgcagggccagcggagcgacgtgtacagcgacctgaacacccagaggccctactacaagaggcggaaaaggtctgggagtggggctaccaatttctctctcctcaagcaagccggagacgttgaggaaaaccctggacccatgggctggatccggggacggaggagccggcacagctgggagatgagcgagttccacaactacaacctggacctgaagaagagcgacttcagcacccggtggcagaagcagcggtgccccgtggtgaagagcaagtgccgggagaacgccagccccttcttcttctgctgcttcatcgccgtggctatgggcatccggttcatcatcatggtggccatctggagcgccgtgttcctgaacagcctgttcaaccaggaggtgcagatccccctgaccgagagctactgcggcccctgccccaagaactggatctgctacaagaacaactgctaccagttcttcgacgagagcaagaactggtacgagagccaggccagctgcatgagccagaacgccagcctgctgaaggtgtacagcaaggaggaccaggacctgctgaagctggtgaagagctaccactggatgggcctggtgcacatccccaccaacggcagctggcagtgggaggacggcagcatcctgagccccaacctgctgaccatcatcgagatgcagaagggcgactgcgccctgtacgccagcagcttcaagggctacatcgagaactgcagcacccccaacacctacatctgcatgcagcggaccgtgagaagaaagagaagcggcagcggcgagggcagaggcagcctgctgacctgcggcgacgtggaggagaaccccggacccatgattcatctcggacatattctctttctcttgctcttgcctgtcgctgccgctcaaacaactcccggagaaagatcttctctccccgctttttatcccggaacatctggatcttgttctggatgtggatctttgtctctccctctcctcgctggactcgtcgcagctgatgctgtcgcttctctcttgattgtcggagctgtctttttgtgtcagaggcggaagtaccggagcaacaagggcgagagccccgtggagcctgccgagccctgccactacagctgtccccgggaggaggagggcagcaccatccccatccaggaggactaccggaagcccgagcctgcctgcagcccc

SEQ ID NO: 79 - DNA, 1632 bp, synthetic construct
  source 1..1632: mol_type = other DNA
atgatccacctgggccacatcctgttcctgctgctgctgcccgtggccgctgcccagaccacccctggcgagcggagcagcctgcctgccttctaccctggcaccagcggcagctgcagcggctgcggcagcctgagcctgcccctgctggccggcctggtggccgccgacgccgtggccagcctgctgatcgtgggcgccgtgttcctgtgcgccaggcccaggcggagccctgcccaggaggacggcaaggtgtacatcaacatgcccggccggggctacttcctgggcaggctggtgcccaggggcaggggcgctgccgaggctgccacccggaagcagcggatcaccgagaccgagagcccctaccaggagctgcagggccagcggagcgacgtgtacagcgacctgaacacccagaggccctactacaagaggcggaaaaggtctgggagtggggctaccaatttctctctcctcaagcaagccggagacgttgaggaaaaccctggacccatgggctggatccggggacggaggagccggcacagctgggagatgagcgagttccacaactacaacctggacctgaagaagagcgacttcagcacccggtggcagaagcagcggtgccccgtggtgaagagcaagtgccgggagaacgccagccccttcttcttctgctgcttcatcgccgtggctatgggcatccggttcatcatcatggtggccatctggagcgccgtgttcctgaacagcctgttcaaccaggaggtgcagatccccctgaccgagagctactgcggcccctgccccaagaactggatctgctacaagaacaactgctaccagttcttcgacgagagcaagaactggtacgagagccaggccagctgcatgagccagaacgccagcctgctgaaggtgtacagcaaggaggaccaggacctgctgaagctggtgaagagctaccactggatgggcctggtgcacatccccaccaacggcagctggcagtgggaggacggcagcatcctgagccccaacctgctgaccatcatcgagatgcagaagggcgactgcgccctgtacgccagcagcttcaagggctacatcgagaactgcagcacccccaacacctacatctgcatgcagcggaccgtgagaagaaagagaagcggcagcggcgagggcagaggcagcctgctgacctgcggcgacgtggaggagaaccccggacctatggctctgcctgtgacagctctgctgctgcctctggctctgctgctgcacgccgctagacccgattataaggacgacgacgacaagcaaacaactcccggagaaagatcttctctccccgctttttatcccggaacatctggatcttgttctggatgtggatctttgtctctccctctcctcgctggactcgtcgcagctgatgctgtcgcttctctcttgattgtcggagctgtctttttgtgtcagaggcggaagtaccggagcaacaagggcgagagccccgtggagcctgccgagccctgccactacagctgtccccgggaggaggagggcagcaccatccccatccaggaggactaccggaagcccgagcctgcctgcagcccc

SEQ ID NO: 80 - protein, 20 aa, synthetic construct
NAVPNLRGDLQVLAQKVART

SEQ ID NO: 81 - protein, 25 aa, synthetic construct
MGWLCSGLLFPVSCLVLLQVASSGN

SEQ ID NO: 82 - protein, 107 aa, synthetic construct
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 83 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 84 - protein, 695 aa, synthetic construct
MGWSCIILFLVATATGVHSQIQLVQSGPELKKPGETVKISCKTSGYTFTDYSMHWVNQAPGKGLKWMGWINTETGEPTYTDDFKGRFAFSLETSASTAYLQINNLKNEDTATYFCARTAVYWGQGTTLTVSSGSTSGSGKPGSGEGSDIQMTQSPSSLSASLGERVSLTCRASQEISGSLSWLQQKPDGTIKRLIYAASTLNSGVPKRFSGRRSGSDYSLTISSLESEDFVDYYCLQYSSYPWSFGGGTKLEIKEPKSPDKTHTCPPCPSHTQPLGVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKFWVLVVVGGVLACYSLLVTVAFIIFWVARPRRSPAQEDGKVYINMPGRGGRLVPRGRGAAEAATRKQRITETESPYQELQGQRSDVYSDLNTQRPYYKRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 85 - protein, 22 aa, synthetic construct
ARPRRSPAQEDGKVYINMPGRG

SEQ ID NO: 86 - protein, 49 aa, synthetic construct
GRLVPRGRGAAEAATRKQRITETESPYQELQGQRSDVYSDLNTQRPYYK

SEQ ID NO: 87 - protein, 360 aa, Homo sapiens
MEDFNMESDSFEDFWKGEDLSNYSYSSTLPPFLLDAAPCEPESLEINKYFVVIIYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRYLVKFICLSIWGLSLLLALPVLLFRRTVYSSNVSPACYEDMGNNTANWRMLLRILPQSFGFIVPLLIMLFCYGFTLRTLFKAHMGQKHRAMRVIFAVVLIFLLCWLPYNLVLLADTLMRTQVIQETCERRNHIDRALDATEILGILHSCLNPLIYAFIGQKFRHGLLKILAIHGLISKDSLPKDSRPSFVGSSSGHTSTTL

SEQ ID NO: 88 - DNA, 1080 bp, synthetic construct
  source 1..1080: mol_type = other DNA
atggaggatttcaatatggagagcgactccttcgaggatttttggaagggcgaggacctgtctaactacagctatagctccacactgcccccttttctgctggatgccgccccttgtgagccagagtccctggagatcaacaagtacttcgtggtcatcatctatgccctggtgtttctgctgtctctgctgggcaatagcctggtcatgctggtcatcctgtactccagggtgggccgctctgtgaccgacgtgtatctgctgaatctggccctggccgatctgctgttcgcactgacactgccaatctgggcagcaagcaaggtgaacggctggatcttcggcacctttctgtgcaaggtggtgtctctgctgaaggaggtgaacttctacagcggcatcctgctgctggcctgtatctccgtggaccggtatctggccatcgtgcacgccaccaggacactgacccagaagcggtacctggtgaagttcatctgcctgagcatctggggactgtccctgctgctggccctgcctgtgctgctgtttaggcgcacagtgtactctagcaacgtgtctccagcctgttatgaggatatgggcaacaataccgccaattggaggatgctgctgcgcatcctgccacagagcttcggctttatcgtgcccctgctgatcatgctgttctgctacggctttacactgcggaccctgttcaaggcccacatgggccagaagcaccgggccatgagagtgatcttcgccgtggtgctgatctttctgctgtgctggctgccctataacctggtgctgctggccgacacactgatgcggacccaggtcatccaggagacatgcgagcggagaaaccacatcgacagagccctggatgccaccgagatcctgggcatcctgcactcctgtctgaatcctctgatctatgccttcatcggccagaagtttaggcacggcctgctgaagatcctggccatccacggcctgatctccaaggactctctgcccaaggatagccgcccttccttcgtgggctcctctagcggccacacctctaccacactg

SEQ ID NO: 89 - DNA, 8670 bp, synthetic construct
  source 1..8670: mol_type = other DNA
gatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgattgtgtgtatgtatgtttgtgtgtgattgtgtgtgtgtgattgtgcatgtgtgtgtgtgtgattgtgtttatgtgtatgattgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagaagccagtttgcatctgtcaggatcaatttcccattatgccagtcatattaattactagtcaattagttgatttttatttttgacatatacatgtgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgatccacctgggccacatcctgttcctgctgctgctgcccgtggccgctgcccagaccacccctggcgagcggagcagcctgcctgccttctaccctggcaccagcggcagctgcagcggctgcggcagcctgagcctgcccctgctggccggcctggtggccgccgacgccgtggccagcctgctgatcgtgggcgccgtgttcctgtgcgccaggcccaggcggagccctgcccaggaggacggcaaggtgtacatcaacatgcccggccggggctacttcctgggcaggctggtgcccaggggcaggggcgctgccgaggctgccacccggaagcagcggatcaccgagaccgagagcccctaccaggagctgcagggccagcggagcgacgtgtacagcgacctgaacacccagaggccctactacaagaggcggaaaaggtctgggagtggggctaccaatttctctctcctcaagcaagccggagacgttgaggaaaaccctggacccatgggctggatccggggacggaggagccggcacagctgggagatgagcgagttccacaactacaacctggacctgaagaagagcgacttcagcacccggtggcagaagcagcggtgccccgtggtgaagagcaagtgccgggagaacgccagccccttcttcttctgctgcttcatcgccgtggctatgggcatccggttcatcatcatggtggccatctggagcgccgtgttcctgaacagcctgttcaaccaggaggtgcagatccccctgaccgagagctactgcggcccctgccccaagaactggatctgctacaagaacaactgctaccagttcttcgacgagagcaagaactggtacgagagccaggccagctgcatgagccagaacgccagcctgctgaaggtgtacagcaaggaggaccaggacctgctgaagctggtgaagagctaccactggatgggcctggtgcacatccccaccaacggcagctggcagtgggaggacggcagcatcctgagccccaacctgctgaccatcatcgagatgcagaagggcgactgcgccctgtacgccagcagcttcaagggctacatcgagaactgcagcacccccaacacctacatctgcatgcagcggacagtgcggagaaagagatccggatctggagagggaagaggaagcctgctgacctgcggcgacgtggaggagaacccaggacccatggaggatttcaatatggagagcgactccttcgaggatttttggaagggcgaggacctgtctaactacagctatagctccacactgcccccttttctgctggatgccgccccttgtgagccagagtccctggagatcaacaagtacttcgtggtcatcatctatgccctggtgtttctgctgtctctgctgggcaatagcctggtcatgctggtcatcctgtactccagggtgggccgctctgtgaccgacgtgtatctgctgaatctggccctggccgatctgctgttcgcactgacactgccaatctgggcagcaagcaaggtgaacggctggatcttcggcacctttctgtgcaaggtggtgtctctgctgaaggaggtgaacttctacagcggcatcctgctgctggcctgtatctccgtggaccggtatctggccatcgtgcacgccaccaggacactgacccagaagcggtacctggtgaagttcatctgcctgagcatctggggactgtccctgctgctggccctgcctgtgctgctgtttaggcgcacagtgtactctagcaacgtgtctccagcctgttatgaggatatgggcaacaataccgccaattggaggatgctgctgcgcatcctgccacagagcttcggctttatcgtgcccctgctgatcatgctgttctgctacggctttacactgcggaccctgttcaaggcccacatgggccagaagcaccgggccatgagagtgatcttcgccgtggtgctgatctttctgctgtgctggctgccctataacctggtgctgctggccgacacactgatgcggacccaggtcatccaggagacatgcgagcggagaaaccacatcgacagagccctggatgccaccgagatcctgggcatcctgcactcctgtctgaatcctctgatctatgccttcatcggccagaagtttaggcacggcctgctgaagatcctggccatccacggcctgatctccaaggactctctgcccaaggatagccgcccttccttcgtgggctcctctagcggccacacctctaccacactgtgacagccactcgag

SEQ ID NO: 90 - protein, 774 aa, synthetic construct
MIHLGHILFLLLLPVAAAQTTPGERSSLPAFYPGTSGSCSGCGSLSLPLLAGLVAADAVASLLIVGAVFLCARPRRSPAQEDGKVYINMPGRGYFLGRLVPRGRGAAEAATRKQRITETESPYQELQGQRSDVYSDLNTQRPYYKRRKRSGSGATNFSLLKQAGDVEENPGPMGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVRRKRSGSGEGRGSLLTCGDVEENPGPMEDFNMESDSFEDFWKGEDLSNYSYSSTLPPFLLDAAPCEPESLEINKYFVVIIYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRYLVKFICLSIWGLSLLLALPVLLFRRTVYSSNVSPACYEDMGNNTANWRMLLRILPQSFGFIVPLLIMLFCYGFTLRTLFKAHMGQKHRAMRVIFAVVLIFLLCWLPYNLVLLADTLMRTQVIQETCERRNHIDRALDATEILGILHSCLNPLIYAFIGQKFRHGLLKILAIHGLISKDSLPKDSRPSFVGSSSGHTSTTL

SEQ ID NO: 91 - DNA, 2337 bp, synthetic construct
  source 1..2337: mol_type = other DNA
atgatccacctgggccacatcctgttcctgctgctgctgcccgtggccgctgcccagaccacccctggcgagcggagcagcctgcctgccttctaccctggcaccagcggcagctgcagcggctgcggcagcctgagcctgcccctgctggccggcctggtggccgccgacgccgtggccagcctgctgatcgtgggcgccgtgttcctgtgcgccaggcccaggcggagccctgcccaggaggacggcaaggtgtacatcaacatgcccggccggggctacttcctgggcaggctggtgcccaggggcaggggcgctgccgaggctgccacccggaagcagcggatcaccgagaccgagagcccctaccaggagctgcagggccagcggagcgacgtgtacagcgacctgaacacccagaggccctactacaagaggcggaaaaggtctgggagtggggctaccaatttctctctcctcaagcaagccggagacgttgaggaaaaccctggacccatgggctggatccggggacggaggagccggcacagctgggagatgagcgagttccacaactacaacctggacctgaagaagagcgacttcagcacccggtggcagaagcagcggtgccccgtggtgaagagcaagtgccgggagaacgccagccccttcttcttctgctgcttcatcgccgtggctatgggcatccggttcatcatcatggtggccatctggagcgccgtgttcctgaacagcctgttcaaccaggaggtgcagatccccctgaccgagagctactgcggcccctgccccaagaactggatctgctacaagaacaactgctaccagttcttcgacgagagcaagaactggtacgagagccaggccagctgcatgagccagaacgccagcctgctgaaggtgtacagcaaggaggaccaggacctgctgaagctggtgaagagctaccactggatgggcctggtgcacatccccaccaacggcagctggcagtgggaggacggcagcatcctgagccccaacctgctgaccatcatcgagatgcagaagggcgactgcgccctgtacgccagcagcttcaagggctacatcgagaactgcagcacccccaacacctacatctgcatgcagcggacagtgcggagaaagagatccggatctggagagggaagaggaagcctgctgacctgcggcgacgtggaggagaacccaggacccatggaggatttcaatatggagagcgactccttcgaggatttttggaagggcgaggacctgtctaactacagctatagctccacactgcccccttttctgctggatgccgccccttgtgagccagagtccctggagatcaacaagtacttcgtggtcatcatctatgccctggtgtttctgctgtctctgctgggcaatagcctggtcatgctggtcatcctgtactccagggtgggccgctctgtgaccgacgtgtatctgctgaatctggccctggccgatctgctgttcgcactgacactgccaatctgggcagcaagcaaggtgaacggctggatcttcggcacctttctgtgcaaggtggtgtctctgctgaaggaggtgaacttctacagcggcatcctgctgctggcctgtatctccgtggaccggtatctggccatcgtgcacgccaccaggacactgacccagaagcggtacctggtgaagttcatctgcctgagcatctggggactgtccctgctgctggccctgcctgtgctgctgtttaggcgcacagtgtactctagcaacgtgtctccagcctgttatgaggatatgggcaacaataccgccaattggaggatgctgctgcgcatcctgccacagagcttcggctttatcgtgcccctgctgatcatgctgttctgctacggctttacactgcggaccctgttcaaggcccacatgggccagaagcaccgggccatgagagtgatcttcgccgtggtgctgatctttctgctgtgctggctgccctataacctggtgctgctggccgacacactgatgcggacccaggtcatccaggagacatgcgagcggagaaaccacatcgacagagccctggatgccaccgagatcctgggcatcctgcactcctgtctgaatcctctgatctatgccttcatcggccagaagtttaggcacggcctgctgaagatcctggccatccacggcctgatctccaaggactctctgcccaaggatagccgcccttccttcgtgggctcctctagcggccacacctctaccacactgtgacagccactcgag

SEQ ID NO: 92 - protein, 329 aa, synthetic construct
MRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRMGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 93 - DNA, 990 bp, synthetic construct
  source 1..990: mol_type = other DNA
atgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgatgcattgcacatgcaggccctgccccctcgcatgggctggatccgcggccgcaggagccggcacagctgggagatgagcgagttccacaactacaacctggacctgaagaagagcgacttcagcacccggtggcagaagcagcggtgccccgtggtgaagagcaagtgccgggagaacgccagccccttcttcttctgctgcttcatcgccgtggctatgggcatccggtttataatcatggtggccatctggagcgccgtgttcctgaacagcctgttcaaccaggaggtgcagatccccctgaccgagagctactgcggcccctgccccaagaactggatctgctacaagaacaactgctaccagttcttcgacgagagcaagaactggtacgagagccaggccagctgcatgagccagaacgccagcctgctgaaggtgtacagcaaggaggaccaggacctgctgaagctggtgaagagctaccactggatgggcctggtgcacatccccaccaacggcagctggcagtgggaggacggcagcatcctgagccccaacctgctgaccatcatcgagatgcagaagggcgactgcgccctgtacgccagcagcttcaagggctacatcgagaactgcagcacccccaacacctacatctgcatgcagcggaccgtgtaa

SEQ ID NO: 94 - DNA, 1350 bp, synthetic construct
  source 1..1350: mol_type = other DNA
atgatccacctgggccacatcctgttcctgctgctgctgcccgtggccgctgcccagaccacccctggcgagcggagcagcctgcctgccttctaccctggcaccagcggcagctgcagcggctgcggcagcctgagcctgcccctgctggccggcctggtggccgccgacgccgtggccagcctgctgatcgtgggcgccgtgttcctgtgcgccaggcccaggcggagccctgcccaggaggacggcaaggtgtacatcaacatgcccggccggggcaggcggaaaaggtctgggagtggggctaccaatttctctctcctcaagcaagccggagacgttgaggaaaaccctggacccatgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgatgcattgcacatgcaggccctgccccctcgcatgggctggatccgcggccgcaggagccggcacagctgggagatgagcgagttccacaactacaacctggacctgaagaagagcgacttcagcacccggtggcagaagcagcggtgccccgtggtgaagagcaagtgccgggagaacgccagccccttcttcttctgctgcttcatcgccgtggctatgggcatccggtttataatcatggtggccatctggagcgccgtgttcctgaacagcctgttcaaccaggaggtgcagatccccctgaccgagagctactgcggcccctgccccaagaactggatctgctacaagaacaactgctaccagttcttcgacgagagcaagaactggtacgagagccaggccagctgcatgagccagaacgccagcctgctgaaggtgtacagcaaggaggaccaggacctgctgaagctggtgaagagctaccactggatgggcctggtgcacatccccaccaacggcagctggcagtgggaggacggcagcatcctgagccccaacctgctgaccatcatcgagatgcagaagggcgactgcgccctgtacgccagcagcttcaagggctacatcgagaactgcagcacccccaacacctacatctgcatgcagcggaccgtgtaa

SEQ ID NO: 95 - protein, 135 aa, Homo sapiens; identical to SEQ ID NO: 16

SEQ ID NO: 96 - protein, 144 aa, Homo sapiens; identical to SEQ ID NO: 15

SEQ ID NO: 97 - protein, 127 aa, synthetic construct
PLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 98 - protein, 23 aa, Mus musculus
VVRVLAIALAIRFTLNTLMWLAI

SEQ ID NO: 99 - protein, 37 aa, Mus musculus
KISPMFVVRVLAIALAIRFTLNTLMWLAIFKETFQPV

SEQ ID NO: 100 - protein, 215 aa, Rattus sp.
MSKCHNYDLKPAKWDTSQEHQKQRSALPTSRPGENGIIRRRSSIEELKISPLFVVRVLVAAMTIRFTVITLTWLAVFITLLCNKEVSVSSREGYCGPCPNDWICHRNNCYQFFNENKAWNQSQASCLSQNSSLLKIYSKEEQDFLKLVKSYHWMGLVQSPANGSWQWEDGSSLSPNELTLVKTPSGTCAVYGSSFKAYTEDCSNPNTYICMKRAV

SEQ ID NO: 101 - protein, 23 aa, Rattus sp.
LFVVRVLVAAMTIRFTVITLTWL

SEQ ID NO: 102 - protein, 440 aa, synthetic construct
MALPVTALLLPLALLLHAARPDYKDDDDKLRPVQAQAQSDCSCSTVSPGVLAGIVMGDLVLTVLIALAVYFLGRLVPRGRGAAEAATRKQRITETESPYQELQGQRSDVYSDLNTQRPYYKRRKRSGSGEGRGSLLTCGDVEENPGPMIHLGHILFLLLLPVAAAQTTPGERSSLPAFYPGTSGSCSGCGSLSLPLLAGLVAADAVASLLIVGAVFLCARPRRSPAQEDGKVYINMPGRGRRKRSGSGATNFSLLKQAGDVEENPGPMKISPMFVVRVLAIALAIRFTLNTLMWLAIFKETFQPVLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 103 - protein, 1320 aa, synthetic construct
ATGGCTCTGCCTGTGACAGCTCTGCTGCTGCCTCTGGCTCTGCTGCTGCACGCCGCTAGACCCGATTATAAGGACGACGACGACAAGCTGAGACCCGTGCAGGCCCAGGCCCAGAGCGACTGCAGCTGCAGCACCGTGAGCCCCGGCGTGCTGGCCGGCATCGTGATGGGCGACCTGGTGCTGACCGTGCTCATCGCCCTTGCCGTGTACTTCCTGGGCAGACTGGTCCCCAGGGGCAGAGGAGCTGCCGAGGCCGCTACCAGAAAGCAGAGGATCACCGAGACAGAGAGCCCCTACCAGGAGCTGCAGGGCCAGAGATCCGACGTGTACAGCGACCTCAACACCCAGAGACCCTATTACAAGAGGCGGAAGCGCTCCGGCTCCGGCGAGGGCCGCGGCAGCCTGCTGACCTGCGGCGACGTGGAAGAGAACCCCGGACCCATGATCCACCTGGGCCACATCCTGTTCCTGCTGCTGCTGCCCGTGGCCGCTGCCCAAACAACACCCGGCGAGAGATCCTCCTTGCCCGCTTTCTATCCCGGAACATCCGGAAGCTGTTCCGGATGTGGATCCCTTTCTTTGCCTTTGCTTGCTGGATTGGTCGCAGCTGACGCTGTCGCTTCCCTCCTTATTGTCGGAGCTGTCTTCCTGTGCGCCAGGCCCAGGCGGAGCCCTGCCCAGGAGGACGGCAAGGTGTACATCAACATGCCCGGCCGGGGCAGGCGGAAGCGCTCCGGGAGTGGGGCTACCAATTTCTCTCTCCTCAAGCAAGCCGGAGACGTTGAGGAAAACCCTGGACCCATGAAAATATCTCCAATGTTCGTTGTTCGAGTCCTTGCTATAGCCTTGGCAATTCGATTCACCCTTAACACATTGATGTGGCTTGCCATTTTCAAAGAGACGTTTCAGCCAGTACTGTTCAACCAGGAGGTGCAGATCCCCCTGACCGAGAGCTACTGCGGCCCCTGCCCAAAAAATTGGATCTGCTACAAGAACAACTGCTACCAGTTCTTCGACGAGAGCAAGAACTGGTACGAGAGCCAGGCCAGCTGCATGAGCCAGAACGCCAGCCTGCTGAAGGTGTACAGCAAGGAGGACCAGGACCTGCTGAAGCTGGTGAAGAGCTACCACTGGATGGGCCTGGTGCACATCCCCACCAACGGCAGCTGGCAGTGGGAGGACGGCAGCATCCTGAGCCCCAACCTGCTGACCATCATCGAGATGCAGAAGGGCGACTGCGCCCTGTACGCCAGCAGCTTCAAGGGCTACATCGAGAACTGCAGCACCCCCAACACCTACATCTGCATGCAGCGGACCGTG

SEQ ID NO: 104 - protein, 15 aa, Homo sapiens
EPKSCDKTHTCPPCP

SEQ ID NO: 105 - protein, 449 aa, synthetic construct
MIHLGHILFLLLLPVAAAQTTPGERSSLPAFYPGTSGSCSGCGSLSLPLLAGLVAADAVASLLIVGAVFLCARPRRSPAQEDGKVYINMPGRGRRKRSGSGATNFSLLKQAGDVEENPGPMRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRMGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 106 - protein, 4 aa, synthetic construct
GGGG

SEQ ID NO: 107 - protein, 5 aa, synthetic construct
GGGGG

SEQ ID NO: 108 - protein, 4 aa, synthetic construct
  REGION 1..4: note = The entire sequence of amino acids 1-4 can be repeated one or more times.
  REGION 1..2: note = The "GG" residues can be repeated one or more times.
  REGION 3..4: note = The "SS" residues can be repeated one or more times.
GGSS

SEQ ID NO: 109 - protein, 4 aa, synthetic construct
  REGION 1..4: note = The entire sequence of amino acids 1-4 can be repeated one or more times.
  REGION 1..2: note = The "SS" residues can be repeated one or more times.
  REGION 3..4: note = The "GG" residues can be repeated one or more times.
SSGG

SEQ ID NO: 110 - protein, 5 aa, synthetic construct
  REGION 1..5: note = The entire sequence of amino acids 1-5 can be repeated one or more times.
GSGGG

SEQ ID NO: 111 - protein, 4 aa, synthetic construct
  REGION 1..4: note = The entire sequence of amino acids 1-4 can be repeated one or more times.
SGGG

SEQ ID NO: 112 - protein, 5 aa, synthetic construct
  REGION 1..5: note = The entire sequence of amino acids 1-5 can be repeated 1 or 20 times.
GGGGS

SEQ ID NO: 113 - protein, 5 aa, synthetic construct
  REGION 1..5: note = The entire sequence of amino acids 1-5 can be repeated 1 or 20 times.
GPPGS

SEQ ID NO: 114 - protein, 5 aa, synthetic construct
  REGION 1..5: note = The entire sequence of amino acids 1-5 can be repeated one or more times.
EAAAK

(19) United States
(12) Patent Application Publication
(10) Pub. No.: US 2026/0335439 A1
(43) Pub. Date: Oct. 29, 2026

(54) COMPOSITIONS COMPRISING NKG2D, CXCR2, AND DAP10/DAP12 FUSION POLYPEPTIDES AND METHODS OF USE THEREOF

(71) Applicant: King's College London, Strand, London (GB)

(72) Inventors: John Maher, Strand, London (GB); David Marc Davies, Strand, London (GB); Daniel Larcombe-Young, Strand, London (GB)

(73) Assignee: King's College London, Strand, London (GB)

(21) Appl. No.: 19/115,718

(22) PCT Filed: Sep. 27, 2023
(86) PCT No.: PCT/EP2023/076759

§ 371 Date: Mar. 26, 2025

(30) Foreign Application Priority Data
Sep. 27, 2022 (GB) 2214120.4

(51) Int. Cl. A61K 40/35; A61K 40/11; A61K 40/15; A61P 35/00; C07K 14/705; C07K 14/715; C12N 5/0783

(52) U.S. Cl. CPC A61K 40/35; A61K 40/11; A61K 40/15; A61P 35/00; C07K 14/7056; C07K 14/7158; C12N 5/0636; C12N 2501/51; C12N 2501/515; C12N 2510/00

(57) ABSTRACT

This invention relates to methods of making immunoresponsive cells, immunoresponsive cells thereof, pharmaceutical compositions, kits, uses thereof and methods of treatment. In particular, there is provided a method of making an immunoresponsive cell, wherein the method comprises (a) genetically modifying an immune cell to express an NKG2D polypeptide; wherein the immune cell has been activated with anti-CD3 and anti-CD28 antibodies, or fragments thereof, conjugated to a nanomatrix; or (b) activating an immune cell, wherein the immune cell has been genetically modified to express an NKG2D polypeptide and wherein activation is with anti-CD3 and anti-CD28 antibodies, or fragments thereof, conjugated to a nanomatrix.

### TECHNICAL FIELD

This invention relates to methods of making immunoresponsive cells, immunoresponsive cells thereof, pharmaceutical compositions, kits, uses thereof and methods of treatment.

### BACKGROUND

Immunotherapy using chimeric antigen receptor (CAR)-engineered T-cells has proven transformative in the management of B-cell malignancy and multiple myeloma. However, application of this technology to solid tumour immunotherapy is impeded by the lack of tumour-selective targets. Most tumour antigens are intracellular and thus cannot easily be recognized by CAR T-cells. Consequently, most solid tumour directed CARs that are currently under development engage targets that are upregulated in tumour cells, but which are found at lower levels in normal tissues.

One of the few target groups that exhibits a high degree of tumour selectivity are the NKG2D ligands. In man, these comprise a group of 8 stress-induced proteins (MICA, MICB, ULBP1-6) that are aberrantly expressed on virtually all tumour cell types. Moreover, NKG2D ligands are also found on tumour associated stromal elements such as endothelium, regulatory T-cells and myeloid derived suppressor cells (Parihar, R., et al., 2019, Cancer Immunol. Res. 7 (3): 363-375; Schmiedel & Mandelboim, 2018, Front. Immunol. (9) 2040). Mice that are genetically deficient in NKG2D demonstrate impaired immunosurveillance for both epithelial and lymphoid malignancies. Evidence that NKG2D ligands are safe therapeutic targets is supported by the fact that they are not found in healthy tissues. Clinical trials using autologous NKG2D-targeted CARs have not revealed any significant safety issues (see https://pubmed.ncbi.nlm.nih.gov/30396908/).

The NKG2D receptor is naturally expressed by natural killer (NK) and some T-cell populations. Each NKG2D homodimer associates with two homodimeric DAP10 adaptor molecules via complementary charged amino acids within the plasma membrane. This interaction is required for cell surface expression and function of NKG2D. DAP10 resembles CD28 in its ability to provide co-stimulation via phosphatidylinositol 3-kinase but, importantly, it lacks a p56lck binding motif that promotes the unwanted recruitment of regulatory T-cells (Kofler, et al., 2011, Mol. Ther. 19:760-767). Potency of DAP10 co-stimulation is underscored by its continued ability to signal following internalization. However, since DAP10 lacks an immunoreceptor tyrosine-based activation motif (ITAM), NKG2D engagement does not lead to full T-cell activation.

A variety of CARs have been developed which use different methods to provide ITAM-dependent signal 1 in addition to co-stimulation (also known as signal 2), as both signal types are necessary to elicit full T-cell activation. The first NKG2D-targeted CAR was developed by Sentman et al. and consists of a fusion of NKG2D to CD37 (Zhang et al., 2005, Blood 106:1544-1551). Although nominally a first-generation CAR, it associates with endogenous DAP10 in T-cells, meaning that both signals 1 and 2 are provided. This CAR is currently undergoing clinical development by Celyad Oncology as Cyad-01. More recently, Chang et al. (2013, Cancer Res. 73:1777-1786) engineered NK cells to co-express an identical CAR in addition to exogenous DAP10. Two further NKG2D CARs have also been described that incorporate either 4-1BB (Song et al., 2013, Hum. Gene Ther. 24:295-305) or CD28 (Lehner et al., 2012, PLoS One 7:e31210) to provide alternative forms of co-stimulation instead of that provided by DAP10. All of these CARs have enabled T-cell mediated tumour cell killing accompanied by cytokine production while the CAR described by Chang et al. also demonstrated transient in vivo anti-tumour activity.

Ligands of the CXCR2 receptor include chemokines of the Cysteine-X-Cysteine (CXC) family that contain an (Glu-Leu-Arg) ELR motif, namely CXCL1-3 and CXCL5-8. Production of these chemokines within tumours is not only undertaken by malignant cells, but also by stromal elements such as fibroblasts and macrophages (Thuwajit et al., 2018, Med Res Rev. 38:1235-54; Thongchot et al, 2021, Int J Oncol. 58:14). Moreover, tumour cells can educate stromal cells to produce these factors, thereby enabling disease progression and resistance to cytotoxic chemotherapy (Le Naour, 2020, J Mol Cell Biol. 12:202-15).

The best studied member of the CXC family is CXCL8, also known as interleukin (IL)-8. Levels of circulating CXCL8 are elevated in patients with certain cancers, including ovarian tumours (Zhang, et al., 2019, Oncol Lett. 17:2365-9), malignant mesothelioma (Judge, et al. 2016, Ann Surg Oncol. 23:1496-500), pancreatic cancer (Hou, et al. 2018, J Clin Med. 7:502, breast cancer (Milovanovic, et al. 2019, Cytokine 118: 93-98; Autenshlyus, et al. 2021, 35: 20587384211034089), esophageal cancer (Huang, et al. Cancer Biomark. 29:139-149) and head and neck cancer (Rezaei, et al. 2019, 39: 727-739). In addition to CXCL8, high level production of the CXCR2 ligands CXCL1, CXCL3, and CXCL5 has also been described in several cancers.

### SUMMARY OF THE INVENTION

The present invention provides a method of making an immunoresponsive cell. The method comprises:
(a) genetically modifying an immune cell to express an NKG2D polypeptide; wherein the immune cell has been activated with anti-CD3 and anti-CD28 antibodies, or fragments thereof, conjugated to a nanomatrix; or
(b) activating an immune cell, wherein the immune cell has been genetically modified to express an NKG2D polypeptide and wherein activation is with anti-CD3 and anti-CD28 antibodies, or fragments thereof, conjugated to a nanomatrix.

Also provided is a method of making an immunoresponsive cell. The method comprises:
(a) activating an immune cell, wherein activating does not comprise phorbol 12-myristate 13-acetate (PMA), phytohaemagglutinin (PHA) or beads; and
(b) genetically modifying the immune cell to express the NKG2D polypeptide.

The present invention also provides an immunoresponsive cell obtainable by the method of either of the above aspects.

Further provided is an immunoresponsive cell genetically modified to express an NKG2D polypeptide, wherein the immunoresponsive cell has been activated with anti-CD3 and anti-CD28 antibodies, or fragments thereof, conjugated to a nanomatrix.

In addition, the present invention provides a pharmaceutical composition comprising the immunoresponsive cell of the invention and a pharmaceutically or physiologically acceptable diluent and/or carrier.

The present invention also provides a kit comprising the immunoresponsive cell or the pharmaceutical composition of the invention.

Also provided is the immunoresponsive cell or the pharmaceutical composition of the invention for use in the treatment or prevention of a disease, optionally wherein the disease is cancer.

In another aspect, there is provided use of the immunoresponsive cell or the pharmaceutical composition of the invention for (i) therapy or (ii) the treatment of cancer.

The invention also provides a method of treating or preventing cancer in a subject, wherein the method comprises administering to the subject the immunoresponsive cell or the pharmaceutical composition of the invention.

### BRIEF DESCRIPTION OF THE DRAWINGS

One or more embodiments of the invention will now be described, by way of example only, with reference to the accompanying drawings, in which:

FIG. 1 shows schematics of the CAR constructs N1012, N1012_CXCR2, NKG2D and CYAD-01_10. In CYAD-01_10, a replica of Cyad-01 has been co-expressed with additional DAP10.

FIG. 2 shows development over time of s.c. CFPac-1 pancreatic tumour xenografts in mice treated 28 days after tumour inoculation with PBS, high dose (1×10⁷) CAR T-cells (N1012, N1012_CXCR2, CYAD-01 replica or untransduced) or low dose (4×10⁶) CAR T-cells (N1012, or N1012_CXCR2). A shows the average tumour volume per treatment group. Survival of the mice is shown in B. C shows individual mouse plots for the averaged data presented in A.

FIG. 3 shows the improved ability of N1012 and N1012_CXCR2 T-cells to persist and proliferate following repeated rounds of stimulation on the triple-negative breast cancer (TNBC) cell lines MDA-MB_468 and MDA-MB_231 compared to controls.

FIG. 4 shows bioluminescence emission from firefly luciferase (ffLuc)-expressing BxPC3 tumour xenografts in mice treated with N1012_CXCR2, N1012 or untransduced T-cells activated in vitro with different activation stimuli (PHA, TransAct or immobilised anti-CD3 and anti-CD28 antibodies). T-cells were administered i.p. on day 12 after tumour inoculation (indicated by vertical dotted lines). Tumour development is measured as total flux (photons/sec).

FIG. 5 shows the bioluminescence results presented in FIG. 4 but with graphs to directly compare the anti-tumour activity of N1012, N1012_CXCR2 and untransduced T-cells when activated using the same stimulus (PHA, TransAct or immobilised anti-CD3 and anti-CD28 antibodies).

FIG. 6 shows individual plots of the results shown in FIGS. 4 and 5. An additional re-challenge was undertaken on day 64 in mice that had achieved a complete response. This re-challenge is designated by the second broken vertical line.

FIG. 7 shows the CD4 to CD8 ratio (as measured by flow cytometry) of N1012, N1012_CXCR2, untransduced, CYAD-01 and CYAD-01_10 T-cells at the end of the culture period (Day 10-12) following stimulation with either 5 ug/mL phytohaemagglutinin-L (PHA-L) or 10 uL TransAct reagent per 1×10⁶ PBMCs. Activation with TransAct shifted all T-cells to a higher CD4:CD8 ratio. This shift was most apparent for N1012 and N1012_CXCR2 T-cells.

FIG. 8 shows (A) Transduction efficacy of T-cells after activation with PHA or TransAct at the end of the ex vivo culture period (Day 10-12) as assessed by flow cytometry. Transduction was assessed as any cell that stained positive for NKG2D or CXCR2 and was assessed in CD4⁺ T-cells due to the lack of endogenous NKG2D expression; (B) Median fluorescence intensity (MFI) of NKG2D was assessed in CD4⁺ T-cells by flow cytometry at the end of the ex vivo culture period. Similar intensities were expressed per construct between those activated with PHA or TransAct reagent; (C) Median fluorescence intensity of CXCR2 in CD4⁺ T-cells was assessed by flow cytometry at the end of the ex vivo culture period. Similar intensities were expressed in N1012_CXCR2 T-cells regardless of whether they were activated by PHA or TransAct reagent.

FIG. 9 shows the fold expansion of T-cells activated with either 5 ug/ml phytohaemagglutinin-L (PHA-L) or 10 uL TransAct reagent per 1×10⁶ PBMCs. This was calculated by dividing the number of T-cells present at the end of the ex vivo culture period by the number of T-cells initially transduced.

FIG. 10 shows the median fluorescence intensity (MFI, A) and transduction percentage (B) of cell surface NKG2D expression in pan-γδ TCR⁺ CD3⁺ cells following transduction with retroviral vectors that encode N1012 or N1012_CXCR2 and 21 days of expansion. Results for untransduced T-cells are shown for comparison.

FIG. 11 shows (A) Fold γδ T-cell expansions calculated on the basis of % pan-γδ TCR⁺ CD3⁺ cells at day 0 (when cells were transduced with retroviral vectors encoding N1012 or N1012_CXCR2), day 7, day 14 and day 21. (B). % pan-γδ TCR⁺ CD3⁺ cells at day 0, day 7, day 14 and day 21 (n=11).

FIG. 12 shows the results from dose response assays measuring the cytotoxicity of untransduced (UT) and CAR⁺ (N1012 or N1012_CXCR2) γδ T-cells against the triple negative breast cancer cell line MDA-MB-468 (A) and the pancreatic cancer cell line BxPC-3 (B) at a range of effector to target ratios (n=3). Results were compared to untransduced cells.

FIG. 13 shows (A) Restimulation assay measuring cytotoxicity of untransduced (UT) and CAR⁺ (N1012 or N1012_CXCR2) γδ T-cells against the acute myeloid leukaemia (AML) cell THP-1-LT (n=2); (B) Restimulation assay measuring cytotoxicity of untransduced (UT) and CAR⁺ γδ T-cells against MDA-MB-468 cells (n=9); (C) Restimulation assay measuring cytotoxicity of untransduced (UT) and CAR⁺ γδ T-cells against BxPC-3 cells (n=8).

FIG. 14 shows (A) Purity of CAR⁺ γδ T-cells following restimulation on MDA-MB-468 cells; (B) Purity of CAR⁺ γδ T-cells following restimulation on BxPC-3 cells; (C) % of αβ T-cells following restimulation on MDA-MB-468 cells; and (D) % of αβ T-cells following restimulation on BxPC-3 cells. Percentages were calculated from flow cytometry dot plots to indicate marker expression.

FIG. 15 shows anti-tumour efficacy of N1012 or N1012_CXCR2 γδ T-cells in NSG mice engrafted with I.P. BxPC-3-LT tumours. Cryopreserved γδ T-cells were thawed and injected at a dose of 10 million I.P. at day 11 of tumour engraftment. Untransduced γδ T-cells and PBS were used as controls.

FIG. 16 is a survival curve of I.P. BxPC3 tumour xenograft-containing mice treated with PBS or 1×10⁷ CAR T-cells (N1012, N1012_CXCR2, or untransduced).

FIG. 17 is a survival curve showing pooled data from in vivo experiments for CFPac1, BxPC3, Kuramochi, Ovsaho, Mesothelioma, triple-negative breast cancer or SKOV3 tumour xenograft-containing mice treated with PBS or 1×10⁷ CAR T-cells (N1012, N1012_CXCR2, or untransduced).

FIG. 18 shows the transduction (A-B) and expansion (C) of primary human T-cells with NKG2D_CXCR2 following activation with differing concentrations of TransAct™. The expression of NKG2D expression was assessed in CD4⁺ T-cells by flow cytometry three and ten days after transduction and used as a marker of transduction efficiency. Untransduced (UT) CD4⁺ T-cells were used as a negative control for transduction.

FIG. 19 demonstrates the anti-tumour efficacy of N1012 and N1012_CXCR2 T-cells in NSG mice engrafted subcutaneously with either LS180 or SW620 metastatic colorectal carcinoma (mCRC) cells. The T-cells were injected intravenously at a dose of 1×10⁷ and tumour growth monitored by caliper measurement. The average growth per treatment group (A-B) or the tumour growth in individual mice in the SW620 model (C) was compared with untransduced (UT) T-cells or CYAD-01 T-cells.

### DETAILED DESCRIPTION

The present invention provides a method of making an immunoresponsive cell. The method comprises:
(a) genetically modifying an immune cell to express an NKG2D polypeptide; wherein the immune cell has been activated with anti-CD3 and anti-CD28 antibodies, or fragments thereof, conjugated to a nanomatrix; or
(b) activating an immune cell, wherein the immune cell has been genetically modified to express an NKG2D polypeptide and wherein activation is with anti-CD3 and anti-CD28 antibodies, or fragments thereof, conjugated to a nanomatrix.

The present inventors have found that activation of immune cells with anti-CD3 and anti-CD28 antibodies, or fragments thereof, conjugated to a nanomatrix and genetic modification of immune cells to express an NKG2D polypeptide results in immunoresponsive cells with unexpectedly improved functionality. In particular, the resulting immunoresponsive cells have unexpectedly improved anti-tumour activity.

In some embodiments, the method comprises:
(i) activating the immune cell with anti-CD3 and anti-CD28 antibodies, or fragments thereof, conjugated to a nanomatrix; and
(ii) genetically modifying the immune cell to express the NKG2D polypeptide.

Alternatively, the method may comprise:
(i) genetically modifying the immune cell to express the NKG2D polypeptide; and
(ii) activating the immune cell with anti-CD3 and anti-CD28 antibodies, or fragments thereof, conjugated to a nanomatrix.

Where embodiments comprise steps (i) and (ii), it will be appreciated that step (i) occurs before step (ii).

In some embodiments, the method comprises simultaneous activation of the immune cell with anti-CD3 and anti-CD28 antibodies, or fragments thereof, conjugated to a nanomatrix and genetic modification of the immune cell to express the NKG2D polypeptide.

Activation of the immune cell with anti-CD3 and anti-CD28 antibodies, or fragments thereof, conjugated to a nanomatrix, may be for a time period of from about 6 hours to about 504 hours. In some embodiments, activation of the immune cell with anti-CD3 and anti-CD28 antibodies, or fragments thereof, conjugated to a nanomatrix is for a time period of at least about 6 hours, at least about 12 hours, at least about 18 hours, at least about 24 hours, at least about 48 hours, at least about 72 hours, at least about 96 hours, at least about 120 hours, at least about 144 hours or at least about 168 hours. In some embodiments, activation of the immune cell with anti-CD3 and anti-CD28 antibodies, or fragments thereof, conjugated to a nanomatrix is for a time period of no more than about 504 hours, no more than about 480 hours, no more than about 456 hours, no more than about 432 hours, no more than about 408 hours, no more than about 384 hours, no more than about 360 hours, no more than about 336 hours, no more than about 312 hours, no more than about 288 hours, no more than about 264 hours, no more than about 240 hours, no more than about 216 hours, no more than about 192 hours, no more than about 168 hours, no more than about 144 hours, no more than about 120 hours or no more than about 96 hours.

Preferably, activation of the immune cell with anti-CD3 and anti-CD28 antibodies, or fragments thereof, conjugated to a nanomatrix is for a time period of at least about 12 hours. More preferably, activation of the immune cell with anti-CD3 and anti-CD28 antibodies, or fragments thereof, conjugated to a nanomatrix is for a time period of at least about 24 hours. Most preferably, activation of the immune cell with anti-CD3 and anti-CD28 antibodies, or fragments thereof, conjugated to a nanomatrix is for a time period of at least about 48 hours.

Preferably, activation of the immune cell with anti-CD3 and anti-CD28 antibodies, or fragments thereof, conjugated to a nanomatrix is for a time period of no more than about 120 hours. More preferably, activation of the immune cell with anti-CD3 and anti-CD28 antibodies, or fragments thereof, conjugated to a nanomatrix is for a time period of no more than about 96 hours. Most preferably, activation of the immune cell with anti-CD3 and anti-CD28 antibodies, or fragments thereof, conjugated to a nanomatrix is for a time period of no more than about 72 hours.

In some embodiments, activation of the immune cell with anti-CD3 and anti-CD28 antibodies, or fragments thereof, conjugated to a nanomatrix is for a time period of from about 12 hours to about 72 hours.

In some embodiments, activation of the immune cell with anti-CD3 and anti-CD28 antibodies, or fragments thereof, conjugated to a nanomatrix is for a time period of about 12 hours. In other embodiments, activation of the immune cell with anti-CD3 and anti-CD28 antibodies, or fragments thereof, conjugated to a nanomatrix is for a time period of about 24 hours. In some embodiments, activation of the immune cell with anti-CD3 and anti-CD28 antibodies, or fragments thereof, conjugated to a nanomatrix is for a time period of about 48 hours.

In some embodiments, the method comprises step (iii) comprising culturing of the immune cell in vitro. In some embodiments, the method comprises:
(i) activating the immune cell with anti-CD3 and anti-CD28 antibodies, or fragments thereof, conjugated to a nanomatrix;
(ii) genetically modifying the immune cell to express the NKG2D polypeptide; and
(iii) culturing the immune cell in vitro.

Culturing the immune cell in vitro may be for a time period of from about 24 hours to about 504 hours. In some embodiments, culturing the immune cell in vitro is for a time period of at least about 24 hours, at least about 48 hours, at least about 72 hours, at least about 96 hours, at least about 120 hours, at least about 144 hours or at least about 168 hours. In some embodiments, culturing the immune cell in vitro is for a time period of no more than about 504 hours, no more than about 480 hours, no more than about 456 hours, no more than about 432 hours, no more than about 408 hours, no more than about 384 hours or no more than about 360 hours.

Preferably, culturing the immune cell in vitro is for a time period of at least about 48 hours. More preferably, culturing the immune cell in vitro is for a time period of at least about 72 hours. Most preferably, culturing the immune cell in vitro is for a time period of at least about 96 hours.

Preferably, culturing the immune cell in vitro is for a time period of no more than about 336 hours. More preferably, culturing the immune cell in vitro is for a time period of no more than about 312 hours. Most preferably, culturing the immune cell in vitro is for a time period of no more than about 288 hours.

In some embodiments, culturing the immune cell in vitro is for a time period of about 168 hours. In other embodiments, culturing the immune cell in vitro is for a time period of about 192 hours. In some embodiments, culturing the immune cell in vitro is for a time period of about 216 hours.

Preferably, culture of the immune cell in vitro is in an appropriate medium. The medium may comprise RPMI-1640 medium or DMEM high glucose medium. Other suitable media will be known to the skilled person. The medium may be supplemented with, for example, antibiotic, IL-2, human AB serum, FBS (foetal bovine serum) or FCS (foetal calf serum) and/or amino acids. Preferably, the medium comprises IL-2. The IL-2 may be recombinant. The IL-2 may be human. In some embodiments the IL-2 is at a concentration in the medium of about 20 U/ml.

In some embodiments, culturing the immune cells in vitro comprises re-activating the immune cells in vitro. The re-activation may comprise activation of the immune cell with anti-CD3 and anti-CD28 antibodies, or fragments thereof, conjugated to a nanomatrix. Alternatively, the re-activation may comprise activation with phorbol 12-myristate 13-acetate (PMA), phytohaemagglutinin (PHA) or anti-CD3 and anti-CD28 antibodies conjugated to beads.

The term “beads”, as used herein, will be well known to the skilled person. Beads conjugated to anti-CD3 and anti-CD28 antibodies are typically inert and of a uniform diameter which is similar to that of antigen-presenting cells. Thus, beads typically have a diameter of from about 1 to about 10 μm in size. The beads are also typically spherical in shape with a non-porous, solid surface area. Such beads have previously commonly been used in in vitro cell culture to activate and expand T-cells. Various such beads are readily commercially available, including, but not limited to Dynabeads® (ThermoFisher Scientific, UK).

In some embodiments, the method does not comprise phorbol 12-myristate 13-acetate (PMA), phytohaemagglutinin (PHA) or beads.

In embodiments comprising steps (i) and (ii), steps (i) and (ii) may not comprise phorbol 12-myristate 13-acetate (PMA), phytohaemagglutinin (PHA) or beads.

### Nanomatrix

As used herein, a “matrix” is a 3D structure comprising a plurality of polymeric chains. The polymeric chains may be interlinked and/or arranged in a criss-cross structure.

In the context of the present invention, the nanomatrix is a flexible nanoscale polymer matrix. The nanomatrix has a non-solid surface area. Typically, the nanomatrix is porous, due to the inclusion of a plurality of polymeric chains. By “flexible”, this will be understood to mean that the nanomatrix is mobile, and thus able to bend. This means that the nanomatrix does not have a solid phase surface; its shape can differ depending on the surrounding environment. The nanomatrix of the invention thus effectively has a mesh or net-like structure.

Preferably, the nanomatrix comprises a matrix of mobile polymer chains conjugated to the anti-CD3 and anti-CD28 antibodies or fragments thereof. In some embodiments, the nanomatrix comprises at least two nanomatrices, with the anti-CD3 antibody or fragment thereof conjugated to a first nanomatrix and the anti-CD28 antibody or fragment thereof conjugated to a second nanomatrix.

The polymer chains may comprise collagen, purified proteins, purified peptides, polysaccharides, glycosaminoglycans, or extracellular matrix compositions.

In some embodiments, the polymer chains comprise polysaccharides. Exemplary polysaccharides may include, but not necessarily be limited to, cellulose, agarose, dextran, chitosan, hyaluronic acid, and alginate. In some embodiments, the polysaccharide comprises dextran. Thus, in some embodiments, the nanomatrix comprises dextran chains.

Other polymers may include polyesters, polyethers, polyanhydrides, polyalkyl cyanoacrylates, polyacrylamides, polyorthoesters, polyphosphazenes, polyvinyl acetates, block copolymers, polypropylene, polytetrafluorethylene (PTFE), or polyurethanes. The polymer may be lactic acid or a copolymer. A copolymer may comprise lactic acid and glycolic acid (PLGA).

The polymer chains may be hydrophilic.

In some embodiments, the nanomatrix has a diameter of from about 1 to about 500 nm.

In some embodiments, the nanomatrix has a diameter of at least about 1 nm, at least about 10 nm, at least about 20 nm, at least about 50 nm or at least about 70 nm. In some embodiments, the nanomatrix has a diameter of no more than about 1000 nm, no more than about 500 nm or no more than about 200 nm.

Preferably, the nanomatrix has a diameter of from about 50 nm to about 200 nm. More preferably, the nanomatrix has a diameter of from about 10 nm to about 200 nm. Most preferably, the nanomatrix has a diameter of about 100 nm.

Preferably, the nanomatrix is biodegradable. Also preferably, the nanomatrix is non-toxic to living cells. By non-toxic to living cells, this will be understood to mean that the nanomatrix does not detrimentally affect the cell viability.

In some embodiments, the nanomatrix is soluble or colloidal. In the context of the present invention, the term “soluble” will be understood to mean that the nanomatrix is dissolvable in a solvent. The solvent may be an aqueous solution, such as a media as described above. As the skilled person will appreciate, the term “colloidal” refers to the nanomatrix being insoluble but suspended throughout a solvent to form a mixture comprising the nanomatrix and the solvent.

The term “antibody”, as used herein, refers to polyclonal or monoclonal antibodies. An antibody fragment may comprise Fab, Fab′, F(ab′)2, Fv and single chain antibodies. These antibodies and antibody fragments can readily be generated by various methods known in the art. The antibody may be of any species, for example murine, sheep or human.

Various anti-CD3 antibodies, anti-CD28 antibodies and fragments thereof are known in the art and readily available. In the context of the present invention, any fragment of an anti-CD3 antibody or anti-CD-28 antibody is a functional fragment, in that it retains the functional activity of the anti-CD3 or anti-CD28 antibody.

In some embodiments, the anti-CD3 antibody or fragment thereof is humanized. In some embodiments, the anti-CD28 antibody or fragment thereof is humanized. In some embodiments, the anti-CD3 antibody or fragment thereof and the anti-CD28 antibody or fragment thereof are humanized. As the skilled person will appreciate, a humanized antibody is an antibody from a non-human species wherein the amino acid sequence of the antibody has been genetically modified to increase similarity to human antibodies, thereby reducing immunogenicity. Methods to humanize antibodies are well known in the art.

By “conjugation” or “conjugated” this will be understood to mean that the antibody (or other agent) is linked or coupled to the nanomatrix. The linkage or coupling may be covalent or non-covalent. Covalent linkages may comprise a linkage to a carboxyl group on a polymer chain. Non-covalent linkages may comprise a biotin-avidin interaction.

The nanomatrix may be conjugated to additional agents to the anti-CD3 and anti-CD28 antibodies, or fragments thereof. Various suitable additional agents will be known to the skilled person. Additional agents may include, but not necessarily be limited to polynucleotides and/or proteins. Proteins may include antibodies, fragments and derivatives thereof, fusion proteins and genetically modified proteins. Preferably, additional agents comprise cell co-stimulatory molecules. Exemplary co-stimulatory molecules include, but are not necessarily limited to anti-CD5, anti-CD4, anti-CD8, anti-MHCI, anti-MHCII, anti CTLA-4, anti-ICOS, anti-PD-1, anti-OX40, anti-CD27L (CD70), anti 4-1BBL, anti CD30L and anti-LIGHT antibodies, fragments or derivatives thereof.

Other additional agents may include cytokines, chemokines, cytokine receptors and/or chemokine receptors. Suitable cytokines may comprise IL-2, IFN-γ, IL-12, IL-17, IL-1 IL-15, IL-4, IL-10 and TNF-α. Exemplary chemokine receptors include, but are not necessarily limited to CCR1, CCR2, CCR3, CCR4, CCR5, and CXCR3.

In some embodiments, an additional agent may comprise any agent capable of binding to cellular adhesion molecules on T-cells such as mAbs, fusion proteins and the corresponding ligands or fractions thereof to adhesion molecules in the following categories: cadherins, ICAM, integrins, and selectins. Examples of adhesion molecules on T-cells are: CD44, CD31, CD18/CD11 a (LFA-1), CD29, CD54 (ICAM-1), CD62L (L-selectin), and CD29/CD49d (VLA-4).

In some embodiments, further agents may be embedded into the nanomatrix. For example, further agents may be embedded into the polymer chains. Such further agents may comprise magnetic agents or fluorescent agents. For example, metal oxide crystals may be embedded into the polymer chains. Preferably, iron oxide crystals are embedded into the polymer chains. Various other suitable magnetic and fluorescent agents are known to those skilled in the art.

By embedded, this will be understood to refer to the agent being located within one or more polymer chains of the nanomatrix. Thus, in such embodiments, the polymer chain comprises the further agent.

In some embodiments, the nanomatrix comprises iron oxide crystals embedded into a biocompatible polysaccharide matrix with a diameter of about 100 nm. In some embodiments, the nanomatrix is colloidal and comprises iron oxide crystals embedded into a biocompatible polysaccharide matrix with a diameter of about 100 nm.

In some embodiments the anti-CD3 and anti-CD28 antibodies, or fragments thereof, conjugated to a nanomatrix is the nanomatrix disclosed in WO2014/048920A1, which is herein incorporated in its entirety. This nanomatrix may be referred to as the MACS GMP TransAct CD3/CD28 Kit (Miltenyi Biotec GmbH, Order no. 170-076-140).

In some embodiments, the anti-CD3 and anti-CD28 antibodies, or fragments thereof, conjugated to a nanomatrix is at a concentration of at least about 0.1 uL per 1×10⁶ cells, at least about 0.5 uL per 1×10⁶ cells, at least about 1 uL per 1×10⁶ immune cells, at least about 2 uL per 1×10⁶ immune cells, at least about 5 uL per 1×10⁶ immune cells or at least about 10 uL per 1×10⁶ immune cells.

In some embodiments, the anti-CD3 and anti-CD28 antibodies, or fragments thereof, conjugated to a nanomatrix is at a concentration of less than about 50 uL per 1×10⁶ immune cells, less than about 40 uL per 1×10⁶ immune cells, less than about 30 uL per 1×10⁶ immune cells or less than about 20 uL per 1×10⁶ immune cells.

Preferably, the anti-CD3 and anti-CD28 antibodies, or fragments thereof, conjugated to a nanomatrix is at a concentration of about 10 uL per 1×10⁶ immune cells.

In some embodiments, the anti-CD3 and anti-CD28 antibodies, or fragments thereof, conjugated to a nanomatrix is at a concentration of about 5 uL per 1×10⁶ immune cells.

In some embodiments, the anti-CD3 and anti-CD28 antibodies, or fragments thereof, conjugated to a nanomatrix is at a concentration of about 1 uL per 1×10⁶ immune cells.

In some embodiments, the anti-CD3 and anti-CD28 antibodies, or fragments thereof, conjugated to a nanomatrix is at a concentration of about 1 to about 5 uL per 1×10⁶ immune cells.

In some embodiments one or more steps of the method are automated. As used herein, the term “automated” refers to the method being automated through use of devices and/or computers and computer softwares, such that once the automated step/method begins, there is no requirement for manual intervention. In some embodiments the method is automated.

In some embodiments one or more steps of the method are performed in a closed and sterile system. In some embodiments the method is performed in a closed and sterile system. By “closed and sterile system”, this will be understood to refer to a closed cell sample processing system which meets international standards for clinical cell processing.

In some embodiments, the immune cell is genetically modified to further express a DAP10/DAP12 fusion polypeptide. Genetic modification to further express a DAP10/DAP12 fusion polypeptide may be simultaneous, prior or after to genetic modification to express an NKG2D polypeptide.

In some embodiments, the method comprises:
(a) genetically modifying an immune cell to express an NKG2D polypeptide and a DAP10/DAP12 fusion polypeptide; wherein the immune cell has been activated with anti-CD3 and anti-CD28 antibodies, or fragments thereof, conjugated to a nanomatrix; or
(b) activating an immune cell, wherein the immune cell has been genetically modified to express an NKG2D polypeptide and a DAP10/DAP12 fusion polypeptide and wherein activation is with anti-CD3 and anti-CD28 antibodies, or fragments thereof, conjugated to a nanomatrix.

In some embodiments, the method comprises:
(i) genetically modifying the immune cell to express the NKG2D polypeptide and a DAP10/DAP12 fusion polypeptide; and
(ii) activating the immune cell with anti-CD3 and anti-CD28 antibodies, or fragments thereof, conjugated to a nanomatrix;
Preferably, the method comprises:
(i) activating the immune cell with anti-CD3 and anti-CD28 antibodies, or fragments thereof, conjugated to a nanomatrix;
(ii) genetically modifying the immune cell to express the NKG2D polypeptide and a DAP10/DAP12 fusion polypeptide.

The fusion polypeptide may have the formula, from N-terminus to C-terminus:

A-B-C-D-E,
wherein
A is an optional N-terminal sequence;
B is a DAP10 polypeptide;
C is an optional linker sequence;
D is a DAP12 polypeptide; and
E is an optional C-terminal sequence.

In some embodiments the fusion polypeptide does not comprise SEQ ID NO: 84. In some embodiments the fusion polypeptide does not comprise an anti-EpCAM peptide. In some embodiments the fusion polypeptide does not comprise SEQ ID NO: 85. In some embodiments the fusion polypeptide does not comprise SEQ ID NO: 86. In some embodiments, the fusion polypeptide does not comprise both SEQ ID NO: 85 and SEQ ID NO: 86.

### DAP10 Polypeptides and Functional Variants Thereof

The DAP10 polypeptide may be mammalian, for example human. Wild-type human DAP10 is encoded by the amino acid sequence having UniProt accession no: Q9UBK5 (SEQ ID NO: 1).

This is a 93 amino acid polypeptide. The first 18 amino acids are considered to be a signal/leader sequence, amino acids 19-48 the extracellular domain, amino acids 49-69 the transmembrane domain, and amino acids 70-93 the cytoplasmic/intracellular domain.

The term “mammal” as used herein includes both humans and non-humans and includes but is not limited to humans, non-human primates, canines, felines, murines, bovines, equines, and porcines.

In some embodiments, the DAP10 polypeptide is a functional variant of DAP10 comprising an amino acid sequence having at least about 80%, at least about 85%, at least about 90%, at least about 95%, at least about 97%, at least about 98% or at least about 99% sequence identity to the DAP10 polypeptide of SEQ ID NO: 1. In some embodiments, the DAP10 polypeptide comprises or consists of an amino acid sequence having the sequence of SEQ ID NO: 1.

The term percent “identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

For purposes herein, percent identity and sequence similarity is performed using the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

Truncated versions of a DAP10 polypeptide may also be used in fusion polypeptides of the invention. In some embodiments, the DAP10 polypeptide is a functional variant of a DAP10 polypeptide which is a truncated version of the polypeptide having the amino acid sequence of SEQ ID NO: 1. In some embodiments, a truncated version of DAP10 comprises only amino acids 19-93 of SEQ ID NO: 1 (i.e. lacking amino acids 1-18, the signal/leader sequence). Such a sequence is referred to as SEQ ID NO: 2 herein. Other truncated versions may comprise amino acids 19-69 of SEQ ID NO: 1, such a sequence comprising merely the extracellular and transmembrane domains of DAP10, and referred to herein as SEQ ID NO: 3. A further truncated version of DAP10 used in the invention may comprise amino acids 1-71 of SEQ ID NO: 1 (i.e. the signal/leader sequence, extracellular domain, transmembrane domain and 2 amino acids from the cytoplasmic/intracellular domain), referred to as SEQ ID NO: 4 herein. A further truncated version of DAP10 used in the invention may comprise amino acids 19-71 of SEQ ID NO: 1 (i.e. the extracellular domain, transmembrane domain and 2 amino acids from the cytoplasmic/intracellular domain), referred to as SEQ ID NO: 5 herein. A further truncated version of DAP10 used in the invention may comprise amino acids 70-93 of SEQ ID NO: 1 (i.e. the intracellular domain), referred to as SEQ ID NO: 6 herein. A yet further truncated version of DAP10 used in the invention may comprise amino acids 49-93 of SEQ ID NO: 1 (i.e. the transmembrane and cytoplasmic/intracellular domains), referred to as SEQ ID NO: 7 herein. A yet further truncated version of DAP10 used in the invention may comprise amino acids 49-69 of SEQ ID NO: 1 (i.e. the transmembrane domain), referred to as SEQ ID NO: 8 herein.

In some embodiments, the truncated version of DAP10 comprises or consists of amino acids 19-93, 19-69, 1-71, 19-71, 19-48, 49-69, 49-93, or 70-93 of SEQ ID NO: 1

Thus, in some embodiments, the DAP10 polypeptide comprises or consists of any one of SEQ ID NOS: 1-8.

As the skilled person will appreciate, a functional variant is a variant of a wild-type protein which retains the functional activity of the wild-type protein. By variant, this will be understood to refer to an amino acid sequence which is altered to that of the wild-type protein. The variant may have at least one, at least two, at least three, at least four, at least five, at least six, at least seven, at least eight, at least nine or at least ten or more point mutations relative to the wild type protein sequence. The point mutation(s) may comprise a substitution, deletion or addition of an amino acid.

In some embodiments, a functional variant of a DAP10 polypeptide retains at least 10% (for example 20%, 30%, 40%, 50%, 60%, 70%, 80%, 90%, 95%, 97%, 98%, 99% or more) of the activity of the wild type polypeptide shown in SEQ ID NO: 1. The activity may be measured by assessment of tyrosine phosphorylation of DAP10 and/or recruitment and activation of the p85 subunit of phosphatidylinositol 3-kinase and the downstream anti-apoptotic kinase, AKT.

### DAP12 Polypeptides and Functional Variants Thereof

The DAP12 polypeptide may be mammalian, for example human. Wild-type human DAP12 is encoded by the amino acid sequence having UniProt accession no: 043914 (SEQ ID NO: 9). The first 21 amino acids are considered to be a signal/leader sequence, amino acids 22-40 the extracellular domain, amino acids 41-61 the transmembrane domain, and amino acids 62-113 the cytoplasmic/intracellular domain.

In some embodiments, the DAP12 polypeptide is a functional variant of DAP12 comprising an amino acid sequence having at least about 80%, at least about 85%, at least about 90%, at least about 95%, at least about 97%, at least about 98% or at least about 99% sequence identity to the DAP12 polypeptide having the sequence of SEQ ID NO: 9. In some embodiments, the DAP12 polypeptide comprises or consists of an amino acid sequence having the sequence of SEQ ID NO: 9.

Truncated versions of a DAP12 polypeptide may also be used in fusion polypeptides of the invention. Thus, in some embodiments the DAP12 polypeptide is a truncated version of the DAP12 polypeptide having the amino acid sequence of SEQ ID NO: 9. An exemplary truncated version of DAP12 comprises only amino acids 22-113 of SEQ ID NO: 9 (i.e. lacking amino acids 1-21, the signal/leader sequence). Such a sequence is referred to as SEQ ID NO: 10 herein. Other truncated versions may comprise amino acids 62-113 of SEQ ID NO: 9, such a sequence comprising merely the cytoplasmic/intracellular domain of DAP12 and referred to herein as SEQ ID NO: 11. Other truncated versions may comprise amino acids 41-61 of SEQ ID NO: 9 (i.e. the transmembrane domain), referred to as SEQ ID NO: 12 herein. Another truncated version may comprise amino acids 22-61 of SEQ ID NO: 9 (i.e., the extracellular and transmembrane domains), referred to as SEQ ID NO: 13 herein.

In some embodiments, the truncated version of DAP12 comprises or consists of amino acids 22-113, 62-113, 22-61, or 41-61 of SEQ ID NO: 9.

In some embodiments, the DAP12 polypeptide comprises or consists of any one of SEQ ID NOs: 9-13.

A functional variant of a DAP12 polypeptide may retain at least 10% (for example 20%, 30%, 40%, 50%, 60%, 70%, 80%, 90%, 95%, 97%, 98%, 99% or more) of the activity of the wild type polypeptide shown in SEQ ID NO: 9. In some embodiments, the activity may be measured using functional assays, such as MTT and measuring cytokine secretion by ELISA.

In some embodiments, the fusion polypeptide comprises or consists of full length human DAP10 fused at its C-terminus to the endodomain of human DAP12 polypeptide, wherein the endodomain is encoded by amino acids 62-113 of human DAP12. In some embodiments the fusion polypeptide has the sequence of SEQ ID NO: 60.

### NKG2D Polypeptides and Functional Variants Thereof

The NKG2D polypeptide may be a mammalian polypeptide. In some embodiments the NKG2D polypeptide is a human polypeptide. Wild-type human NKG2D is encoded by the amino acid sequence having UniProt accession no: P26718 (SEQ ID NO: 14). The wild type NKG2D polypeptide is considered to comprise a cytoplasmic domain (amino acids 1-51), a transmembrane domain (amino acids 52-72) and an extracellular domain (amino acids 73-216).

In some embodiments, the NKG2D polypeptide is a functional variant of NKG2D. The functional variant of NKG2D may comprise an amino acid sequence having at least about 80%, at least about 85%, at least about 90%, at least about 95%, at least about 97%, at least about 98% or at least about 99% sequence identity to the NKG2D polypeptide having the sequence of SEQ ID NO: 14. In some embodiments, the NKG2D polypeptide comprises or consists of SEQ ID NO: 14.

The functional variant of NKG2D may comprise or consist of a truncated version of NKG2D. In some embodiments, the NKG2D polypeptide may be a truncated version of NKG2D comprising only amino acids 73-216 of SEQ ID NO: 14 (i.e., the extracellular domain. Such a sequence is referred to as SEQ ID NO: 15 herein. Other truncated versions may comprise amino acids 82-216 of SEQ ID NO: 14, such a sequence comprising part of the extracellular domain of NKG2D and referred to herein as SEQ ID NO: 16. A further truncated version comprises amino acids 52-216 of SEQ ID NO: 14 (i.e. the transmembrane and extracellular domains), referred to as SEQ ID NO: 17 herein.

A functional variant of a NKG2D polypeptide may retain at least 10% (for example 20%, 30%, 40%, 50%, 60%, 70%, 80%, 90%, 95%, 97%, 98%, 99% or more) of the activity of the wild type polypeptide shown in SEQ ID NO: 14. The activity may be measured using flow cytometry to confirm continued binding to NKG2D ligands and through various cell culture assays (such as MTT and ELISA) aimed at confirming target T-cell lysis, cytokine secretion and co-stimulation.

In some embodiments, the functional variant of the NKG2D polypeptide is a chimeric NKG2D polypeptide. The chimeric NKG2D polypeptide may be a human-murine chimeric polypeptide. The term “murine” refers to a rodent of the subfamily Murinae. The term “murine” comprises rat and mouse.

The term “chimeric NKG2D polypeptide” refers to an NKG2D receptor formed of domains from two or more different organisms. Chimeric NKG2D polypeptides are described in more detail in WO 2021/234163, the disclosure of which is herein incorporated by reference in its entirety.

In some embodiments, the chimeric NKG2D polypeptide comprises from N terminus to C terminus the murine NKG2D transmembrane domain or a variant thereof and a human NKG2D extracellular domain or a variant thereof.

### CXCR2 Polypeptides and Functional Variants Thereof.

In some embodiments, the immune cell is genetically modified to express a CXCR2 polypeptide. Genetic modification to express a CXCR2 polypeptide may be simultaneous, prior to or after genetic modification to express an NKG2D polypeptide.

In some embodiments, the method comprises:
(a) genetically modifying an immune cell to express an NKG2D polypeptide and a CXCR2 polypeptide; wherein the immune cell has been activated with anti-CD3 and anti-CD28 antibodies, or fragments thereof, conjugated to a nanomatrix; or
(b) activating an immune cell, wherein the immune cell has been genetically modified to express an NKG2D polypeptide and a CXCR2 polypeptide and wherein activation is with anti-CD3 and anti-CD28 antibodies, or fragments thereof, conjugated to a nanomatrix.

In some embodiments, the method comprises:
(i) genetically modifying the immune cell to express the NKG2D polypeptide and a CXCR2 polypeptide; and
(ii) activating the immune cell with anti-CD3 and anti-CD28 antibodies, or fragments thereof, conjugated to a nanomatrix;
Preferably, the method comprises:
(i) activating the immune cell with anti-CD3 and anti-CD28 antibodies, or fragments thereof, conjugated to a nanomatrix;
(ii) genetically modifying the immune cell to express the NKG2D polypeptide a CXCR2 polypeptide.

In some embodiments, the method comprises:
(a) genetically modifying an immune cell to express an NKG2D polypeptide, a DAP10/DAP12 fusion polypeptide and a CXCR2 polypeptide; wherein the immune cell has been activated with anti-CD3 and anti-CD28 antibodies, or fragments thereof, conjugated to a nanomatrix; or
(b) activating an immune cell, wherein the immune cell has been genetically modified to express an NKG2D polypeptide, a DAP10/DAP12 fusion polypeptide and a CXCR2 polypeptide and wherein activation is with anti-CD3 and anti-CD28 antibodies, or fragments thereof, conjugated to a nanomatrix.

In some embodiments, the method comprises:
(i) genetically modifying the immune cell to express the NKG2D polypeptide, a DAP10/DAP12 fusion polypeptide and a CXCR2 polypeptide; and
(ii) activating the immune cell with anti-CD3 and anti-CD28 antibodies, or fragments thereof, conjugated to a nanomatrix;
Preferably, the method comprises:
(i) activating the immune cell with anti-CD3 and anti-CD28 antibodies, or fragments thereof, conjugated to a nanomatrix;
(ii) genetically modifying the immune cell to express the NKG2D polypeptide, a DAP10/DAP12 fusion polypeptide and a CXCR2 polypeptide.

Preferably, the CXCR2 polypeptide is a mammalian polypeptide. More preferably, the CXCR2 polypeptide is a human polypeptide. Wild-type human CXCR2 is encoded by the amino acid sequence having UniProt accession no: P25025 (SEQ ID NO: 87).

In some embodiments, the CXCR2 polypeptide comprises a functional variant of the wildtype CXCR2 polypeptide. Thus, the CXCR2 polypeptide may be a functional variant of the CXCR2 polypeptide of SEQ ID NO: 87. The functional variant of the CXCR2 polypeptide may comprise an amino acid sequence having at least about 80%, at least about 85%, at least about 90%, at least about 95%, at least about 97%, at least about 98%, or at least about 99% sequence identity to the CXCR2 polypeptide having the sequence of SEQ ID NO: 87. In some embodiments, the CXCR2 polypeptide comprises or consists of amino acid sequence of SEQ ID NO: 87.

A functional variant of the CXCR2 polypeptide may retain at least 10% (for example, 20%, 30%, 40%, 50%, 60%, 70%, 80%, 90%, 95%, 97%, 98%, 99% or more) of the activity of wild-type human CXCR2 (SEQ ID NO: 87). Activity of the polypeptide may be measured using flow cytometry to confirm binding of CXCR2 to ligand (e.g., IL-8). In some embodiments, activity of the polypeptide is measured through cell culture assays known in the art.

### Linkers

The DAP10 and DAP12 polypeptides of the fusion polypeptide described herein can be directly bonded to each other in a contiguous polypeptide chain or may be indirectly bonded to each other through a suitable linker. Thus, the DAP10/12 fusion polypeptide may comprise a linker sequence.

The linker may be a peptide linker. Peptide linkers are commonly used in fusion polypeptides and methods for selecting or designing linkers are well-known. (See, e.g., Chen X et al., 2013, Adv. Drug Deliv. Rev. 65 (10): 135701369 and Wriggers W et al., 2005, Biopolymers 80:736-746.). Linkers may also be used to join the fusion polypeptide of the disclosure to another polypeptide (such as the NKG2D polypeptide and/or the CXCR2 polypeptide) in a chimeric construct as described further below.

Peptide linkers generally are categorized as i) flexible linkers, ii) helix forming linkers, and iii) cleavable linkers, and examples of each type are known in the art. In one example, a flexible linker is included in the fusion polypeptides described herein. Flexible linkers may contain a majority of amino acids that are sterically unhindered, such as glycine and alanine. The hydrophilic amino acid Ser is also conventionally used in flexible linkers. Examples of flexible linkers include, without limitation: polyglycines (e.g., (Gly)₄ and (Gly)₅), polyalanines poly(Gly-Ala), and poly(Gly-Ser) (e.g., (Glyₙ-Serₙ)ₙ or (Serₙ-Glyₙ)ₙ, wherein each n is independently an integer equal to or greater than 1).

Peptide linkers can be of a suitable length. The peptide linker sequence may be at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, or more amino acid residues in length. For example, a peptide linker can be from about 5 to about 50 amino acids in length; from about 10 to about 40 amino acids in length; from about 15 to about 30 amino acids in length; or from about 15 to about 20 amino acids in length. Variation in peptide linker length may retain or enhance activity, giving rise to superior efficacy in activity studies. The peptide linker sequence may be comprised of naturally or non-naturally occurring amino acids, or a mixture of both naturally and non-naturally occurring amino acids.

In some embodiments, the linker sequence comprises or consists of the amino acids glycine and serine. For example, the linker region may comprise sets of glycine repeats (GSG3)ₙ (SEQ ID NO: 18), where n is a positive integer equal to or greater than 1 (for example 1 to about 20). More specifically, the linker sequence may be GSGGG (SEQ ID NO: 19). The linker sequence may be GSGG (SEQ ID NO: 20). In certain other embodiments, the linker region orientation comprises sets of glycine repeats (SerGly₃)ₙ, where n is a positive integer equal to or greater than 1 (for example 1 to about 20) (SEQ ID NO: 21).

In other embodiments, a linker may contain glycine (G) and serine (S) in a random or a repeated pattern. For example, the linker can be (GGGGS)ₙ (SEQ ID NO: 22), wherein n is an integer ranging from 1 to 20, for example 1 to 4. In a particular example, n is 4 and the linker is GGGGSGGGGSGGGGSGGGGS (SEQ ID NO: 23). In another particular example, n is 3 and the linker is GGGGSGGGGSGGGGS (SEQ ID NO: 24).

In other embodiments, a linker may contain glycine (G), serine (S) and proline (P) in a random or repeated pattern. For example, the linker can be (GPPGS)ₙ, wherein n is an integer ranging from 1 to 20, for example 1-4. In a particular example, n is 1 and the linker is GPPGS (SEQ ID NO: 25).

In general, the linker is not immunogenic when administered in a patient, such as a human. Thus, linkers may be chosen such that they have low immunogenicity or are thought to have low immunogenicity.

The linkers described herein are exemplary, and the linker can include other amino acids, such as Glu and Lys, if desired. The peptide linkers may include multiple repeats of, for example, (G₃S) (SEQ ID NO: 26), (G₄S) (SEQ ID NO: 27), (GYS) (SEQ ID NO: 28), and/or (GlySer) (SEQ ID NO: 29), if desired. The peptide linkers may include multiple repeats of, for example, (SG₄) (SEQ ID NO: 30), (SG₃) (SEQ ID NO: 31), (SG₂) (SEQ ID NO: 32), (SG)₂ (SEQ ID NO: 33) or (SerGly) (SEQ ID NO: 34).

The peptide linkers may include combinations and multiples of repeating amino acid sequence units, such as (G₃S)+(G₄S)+(GlySer) (SEQ ID NO: 26+SEQ ID NO: 27+SEQ ID NO: 29). Alternatively, Ser can be replaced with Ala e.g., (G₄A) (SEQ ID NO: 35) or (G₃A) (SEQ ID NO: 36). In yet other embodiments, the linker comprises the motif (EAAAK)ₙ, where n is a positive integer equal to or greater than 1, for example 1 to about 20 (SEQ ID NO: 37). In certain embodiments, peptide linkers also include cleavable linkers.

The linkers may comprise further domains and/or features, such as a furin cleavage site (RRKR) (SEQ ID NO: 38), a P2A ribosomal skip peptide (ATNFSLLKQAGDVEENPGP) (SEQ ID NO: 39) and/or a T2A ribosomal skip peptide (EGRGSLLTCGDVEENPGP) (SEQ ID NO: 40). Examples of linkers comprising these domains include SGSG+a P2A ribosomal skip peptide (SGSGATNFSLLKQAGDVEENPGP) (SEQ ID NO: 41), SGSG+a T2A ribosomal skip peptide (SGSGEGRGSLLTCGDVEENPGP) (SEQ ID NO: 42), and versions also including a furin cleavage site, i.e. furin cleavage site+SGSG+a P2A ribosomal skip peptide (RRKRSGSGATNFSLLKQAGDVEENPGP) (SEQ ID NO: 43) and furin cleavage site+SGSG+a T2A ribosomal skip peptide (RRKRSGSGEGRGSLLTCGDVEENPGP) (SEQ ID NO: 44). Alternative ribosomal skip peptides that may be used in the invention include F2A (VKQTLNFDLLKLAGDVESNPGP) (SEQ ID NO: 45) and E2A (QCTNYALLKLAGDVESNPGP) (SEQ ID NO: 46).

### N-Terminal Sequences and C-Terminal Sequences

Various sequences may be attached to the N- or C-terminus of the fusion polypeptide, the NKG2D polypeptide and/or the CXCR2 polypeptide disclosed herein. These sequences may be functional, such as signal peptides, purification tags/sequences, or half-life extension moieties, or may simply comprise spacer sequences. Alternatively, they may comprise another function, such as a T-cell stimulatory function.

### Purification Tags and Markers

A variety of tags or markers may be attached to the N- or C-terminus of the fusion polypeptide, the NKG2D polypeptide and/or the CXCR2 polypeptide to assist with purification. For example, a tag may comprise an affinity tag. Examples of such affinity tags are a His-tag, a FLAG-tag, Arg-tag, T7-tag, Strep-tag, S-tag, aptamer-tag, V5 tag, AviTag™, myc epitope tag or any combination of these tags. In some embodiments the affinity tag is a His-tag (usually comprising 5-10 histidine residues), for example a 6His tag (i.e. HHHHHH) (SEQ ID NO: 47). In some embodiments the affinity tag is a FLAG tag (i.e. DYKDDDDK) (SEQ ID NO: 48). In some embodiments, the affinity tag is an AviTag™ (i.e. GLNDIFEAQKIEWHE) (SEQ ID NO: 49). In some embodiments, the affinity tag is a V5 tag (GKPIPNPLLGLDST) (SEQ ID NO: 50) or (IPNPLLGLD) (SEQ ID NO: 51). In some embodiments, the affinity tag is a myc epitope tag recognised by the 9e10 antibody (EQKLISEEDL) (SEQ ID NO: 52). Various other tags are suitable for use and well known in the art.

Combinations of such affinity tags may also be used, either comprising one or more tags at the N-terminus, one or more tags at the C-terminus, or one or more tags at each of the N-terminus and the C-terminus. Examples of such combinations include a His tag (H) combined with an AviTag (A), or a His tag (H) combined with both an AviTag (A) and a FLAG tag (F). The tags may be in either orientation, thus the AviTag/His tag may have the orientation N-AH-C or N-HA-C, while the Avi/His/FLAG tag may have the orientation N-AHF-C, N-FHA-C, etc.

For example, a fusion polypeptide may comprise an “AHF” tag having the sequence “GLNDIFEAQKIEWHEGGHHHHHHDYKDDDDK” (SEQ ID NO: 53). Alternatively, a fusion polypeptide may comprise an “FHA” tag having the sequence “DYKDDDDKHHHHHHGGGLNDIFEAQKIEWHE” (SEQ ID NO: 54).

The CD8a leader sequence (amino acids 1-21 of UniProt: P01732 or a shortened derivative comprising amino acids 1-18), is a commonly used T-cell sequence, and is referred to as SEQ ID NO: 55 herein. Thus, the fusion polypeptide, NKG2D polypeptide and/or CXCR2 polypeptide may comprise SEQ ID NO: 55. In some embodiments, the N terminus of the fusion polypeptide, NKG2D polypeptide and/or CXCR2 polypeptide comprises SEQ ID NO: 55.

### Co-Stimulatory Sequences

Various T-cell co-stimulatory activation sequences are known from previous work to engineer CAR-T-cells. The fusion polypeptide, NKG2D polypeptide and/or CXCR2 polypeptide may further comprise or be fused to a T-cell co-stimulatory activation sequence.

The 4-1BB endodomain (amino acids 214-255 of UniProt: Q07011) may also be used as an N- or C-terminal sequence. The 4-1BB endodomain is referred to as SEQ ID NO: 56 herein. The 4-1BB endodomain may act as a co-stimulatory domain.

The CD27 endodomain (amino acids 213-260 of UniProt: P26842) may also be used as an N- or C-terminal sequence. The CD27 endodomain is referred to as SEQ ID NO: 57 herein. The CD27 endodomain may act as a co-stimulatory domain.

The human IgG1 hinge (amino acids 218-229 or 218-232 of UniProt: PODOX5) may also be used as an N- or C-terminal sequence. The human IgG1 hinge is referred to as SEQ ID NO: 58 or SEQ ID NO: 104.

A truncated CD8a hinge (amino acids 138-182 of Uniprot: P01732) may also be used as an N- or C-terminal sequence. The truncated CD8a hinge is referred to as SEQ ID NO: 59.

In embodiments where the cell is genetically modified to further express a DAP10/12 fusion polypeptide the NKG2D polypeptide and the DAP10/12 fusion polypeptide may be genetically encoded as part of a contiguous chimeric construct. The fusion polypeptide and NKG2D polypeptide may then be separated during translation (e.g. using a ribosomal skip peptide) or by post translation cleavage (e.g. using a furin cleavage site). The fusion polypeptide and NKG2D polypeptide may therefore be joined by an optional linker. Such a linker may comprise a cleavage site to facilitate cleavage.

In embodiments where the cell is genetically modified to further express a CXCR2 polypeptide, the NKG2D polypeptide and the CXCR2 polypeptide may be genetically encoded as part of a contiguous chimeric construct. The CXCR2 polypeptide and NKG2D polypeptide may then be separated during translation (e.g. using a ribosomal skip peptide) or by post translation cleavage (e.g. using a furin cleavage site). The CXCR2 polypeptide and NKG2D polypeptide may therefore be joined by an optional linker. Such a linker may comprise a cleavage site to facilitate cleavage.

In embodiments where the cell is genetically modified to further express a CXCR2 polypeptide and a DAP10/12 fusion polypeptide, the NKG2D polypeptide, DAP10/12 fusion polypeptide and the CXCR2 polypeptide may be genetically encoded as part of a contiguous chimeric construct. The CXCR2 polypeptide, DAP10/12 fusion polypeptide and NKG2D polypeptide may then be separated during translation (e.g. using a ribosomal skip peptide) or by post translation cleavage (e.g. using a furin cleavage site).

### Exemplary Constructs

The present disclosure provides the following exemplary fusion polypeptide constructs in Table 1:

TABLE 1
Exemplary DAP10/DAP12 fusion polypeptide constructs

| Name | Sequence |
|---|---|
| DAP10 (full sequence- | MIHLGHILFLLLLPVAAAQTTPGERSSLPAFYPGTSGSCSGCGSLSLPLLA |
| aa1-93)-DAP12 | GLVAADAVASLLIVGAVFLCARPRRSPAQEDGKVYINMPGRGYFLGRLVP |
| endodomain (aa62- | RGRGAAEAATRKQRITETESPYQELQGQRSDVYSDLNTQRPYYK |
| 113) (SEQ ID NO: | |
| 60) | |
| CD8α leader-FLAG- | MALPVTALLLPLALLLHAARPDYKDDDDKQTTPGERSSLPAFYPGTSGSC |
| DAP10 (extracellular | SGCGSLSLPLLAGLVAADAVASLLIVGAVFYFLGRLVPRGRGAAEAATRK |
| and TM-aa19-69)- | QRITETESPYQELQGQRSDVYSDLNTQRPYYK |
| DAP12 endodomain | |
| (aa62-113) (SEQ ID | |
| NO: 61) | |
| CD8α leader-FLAG- | MALPVTALLLPLALLLHAARPDYKDDDDKEPKSCDKTHTCPLLAGLVAAD |
| human IgG1 hinge | AVASLLIVGAVFLCARPRRSPAQEDGKVYINMPGRGYFLGRLVPRGRGAA |
| (aa 218-229 Uniprot | EAATRKQRITETESPYQELQGQRSDVYSDLNTQRPYYK |
| ref P0DOX5)-DAP10 | |
| (TM and endodomain | |
| aa 49-93)-DAP12 | |
| endodomain (aa 62- | |
| 113) (SEQ ID NO: | |
| 62) | |
| CD8α leader- | MALPVTALLLPLALLLHAARPDYKDDDDKTTTPAPRPPTPAPTIASQPLSLR |
| truncated CD8a | PEACRPAAGGAVHTRGLDFACDLLAGLVAADAVASLLIVGAVFLCARPRR |
| hinge (aa 138-182 | SPAQEDGKVYINMPGRGYFLGRLVPRGRGAAEAATRKQRITETESPYQEL |
| Uniprot reference | QGQRSDVYSDLNTQRPYYK |
| P01732)-DAP10 (TM | |
| and endodomain-aa | |
| 49-93)-DAP12 | |
| endodomain (aa 62- | |
| 113) (SEQ ID NO: | |
| 63) | |

In embodiments comprising fusion polypeptides, as explained above, the fusion polypeptides may be expressed as a single chimeric construct with a NKG2D and optionally a CXCR2 polypeptide, for translation-associated or post-translational cleavage. In such constructs, following expression, the translated polypeptides are cleaved to create the separate polypeptides which then self-associate to form a CAR. In some embodiments, a fusion polypeptide is cleaved from a NKG2D polypeptide. Examples of such constructs are shown in Table 2:

TABLE 2
Exemplary chimeric constructs

| Name | Sequence |
|---|---|
| DAP10 (aa1-93)- | MIHLGHILFLLLLPVAAAQTTPGERSSLPAFYPGTSGSCSGCGSLSLPLLA |
| DAP12 endodomain | GLVAADAVASLLIVGAVFLCARPRRSPAQEDGKVYINMPGRGYFLGRLVP |
| (aa62-113)-Furin | RGRGAAEAATRKQRITETESPYQELQGQRSDVYSDLNTQRPYYKRRKRS |
| cleavage-linker-P2A- | GSGATNFSLLKQAGDVEENPGPMGWIRGRRSRHSWEMSEFHNYNLDLK |
| NKG2D (aa1-216) | KSDFSTRWQKQRCPVVKSKCRENASPFFFCCFIAVAMGIRFIIMVAIWSA |
| (Construct 1) (SEQ | VFLNSLFNQEVQIPLTESYCGPCPKNWICYKNNCYQFFDESKNWYESQA |
| ID NO: 64) (N1012) | SCMSQNASLLKVYSKEDQDLLKLVKSYHWMGLVHIPTNGSWQWEDGSI |
| | LSPNLLTIIEMQKGDCALYASSFKGYIENCSTPNTYICMQRTV |
| CD8α leader-FLAG- | MALPVTALLLPLALLLHAARPDYKDDDDKQTTPGERSSLPAFYPGTSGSC |
| DAP10 (aa19-69)- | SGCGSLSLPLLAGLVAADAVASLLIVGAVFYFLGRLVPRGRGAAEAATRK |
| DAP12 (aa62-113)- | QRITETESPYQELQGQRSDVYSDLNTQRPYYKRRKRSGSGEGRGSLLTC |
| Furin cleavage- | GDVEENPGPMIHLGHILFLLLLPVAAAQTTPGERSSLPAFYPGTSGSCSGC |
| linker-T2A-DAP10 | GSLSLPLLAGLVAADAVASLLIVGAVFLCARPRRSPAQEDGKVYINMPGR |
| (aa1-93)-Furin | GRRKRSGSGATNFSLLKQAGDVEENPGPMGWIRGRRSRHSWEMSEFH |
| cleavage-linker-P2A- | NYNLDLKKSDFSTRWQKQRCPVVKSKCRENASPFFFCCFIAVAMGIRFII |
| NKG2D (aa1-216) | MVAIWSAVFLNSLFNQEVQIPLTESYCGPCPKNWICYKNNCYQFFDESK |
| (Construct 3) (SEQ | NWYESQASCMSQNASLLKVYSKEDQDLLKLVKSYHWMGLVHIPTNGS |
| ID NO: 65) | WQWEDGSILSPNLLTIIEMQKGDCALYASSFKGYIENCSTPNTYICMQRT |
| | V |
| DAP10 (aa1-93)- | MIHLGHILFLLLLPVAAAQTTPGERSSLPAFYPGTSGSCSGCGSLSLPLLA |
| DAP12 endodomain | GLVAADAVASLLIVGAVFLCARPRRSPAQEDGKVYINMPGRGYFLGRLVP |
| (aa62-113)-Furin | RGRGAAEAATRKQRITETESPYQELQGQRSDVYSDLNTQRPYYKRRKRS |
| cleavage-linker-P2A- | GSGATNFSLLKQAGDVEENPGPMGWIRGRRSRHSWEMSEFHNYNLDLK |
| NKG2D (aa1-216)- | KSDFSTRWQKQRCPVVKSKCRENASPFFFCCFIAVAMGIRFIIMVAIWSA |
| Furin cleavage- | VFLNSLFNQEVQIPLTESYCGPCPKNWICYKNNCYQFFDESKNWYESQA |
| linker-T2A-DAP10 | SCMSQNASLLKVYSKEDQDLLKLVKSYHWMGLVHIPTNGSWQWEDGSI |
| (aa1-71)-4-1BB | LSPNLLTIIEMQKGDCALYASSFKGYIENCSTPNTYICMQRTVRRKRSGS |
| endodomain (aa214- | GEGRGSLLTCGDVEENPGPMIHLGHILFLLLLPVAAAQTTPGERSSLPAFY |
| 255) (Construct 8) | PGTSGSCSGCGSLSLPLLAGLVAADAVASLLIVGAVFLCKRGRKKLLYIFK |
| (SEQ ID NO: 66) | QPFMRPVQTTQEEDGCSCRFPEEEEGGCEL |
| DAP10 (aa1-93)- | MIHLGHILFLLLLPVAAAQTTPGERSSLPAFYPGTSGSCSGCGSLSLPLLA |
| DAP12 endodomain | GLVAADAVASLLIVGAVFLCARPRRSPAQEDGKVYINMPGRGYFLGRLVP |
| (aa62-113)-Furin | RGRGAAEAATRKQRITETESPYQELQGQRSDVYSDLNTQRPYYKRRKRS |
| cleavage-linker-P2A- | GSGATNFSLLKQAGDVEENPGPMGWIRGRRSRHSWEMSEFHNYNLDLK |
| NKG2D (aa1-216)- | KSDFSTRWQKQRCPVVKSKCRENASPFFFCCFIAVAMGIRFIIMVAIWSA |
| Furin cleavage- | VFLNSLFNQEVQIPLTESYCGPCPKNWICYKNNCYQFFDESKNWYESQA |
| linker-T2A-CD8a | SCMSQNASLLKVYSKEDQDLLKLVKSYHWMGLVHIPTNGSWQWEDGSI |
| leader (aa1-21)- | LSPNLLTIIEMQKGDCALYASSFKGYIENCSTPNTYICMQRTVRRKRSGS |
| FLAG-DAP10 (aa19- | GEGRGSLLTCGDVEENPGPMALPVTALLLPLALLLHAARPDYKDDDDKQT |
| 71)-4-1BB | TPGERSSLPAFYPGTSGSCSGCGSLSLPLLAGLVAADAVASLLIVGAVFLC |
| endodomain (aa214- | KRGRKKLLYIFKQPFMRPVQTTQEEDGCSCRFPEEEEGGCEL |
| 255) (Construct 9) | |
| (SEQ ID NO: 67) | |
| DAP10 (aa1-93)- | MIHLGHILFLLLLPVAAAQTTPGERSSLPAFYPGTSGSCSGCGSLSLPLLA |
| DAP12 endodomain | GLVAADAVASLLIVGAVFLCARPRRSPAQEDGKVYINMPGRGYFLGRLVP |
| (aa62-113)-Furin | RGRGAAEAATRKQRITETESPYQELQGQRSDVYSDLNTQRPYYKRRKRS |
| cleavage-linker-P2A- | GSGATNFSLLKQAGDVEENPGPMGWIRGRRSRHSWEMSEFHNYNLDLK |
| NKG2D (aa1-216)- | KSDFSTRWQKQRCPVVKSKCRENASPFFFCCFIAVAMGIRFIIMVAIWSA |
| Furin cleavage- | VFLNSLFNQEVQIPLTESYCGPCPKNWICYKNNCYQFFDESKNWYESQA |
| linker-T2A-DAP10 | SCMSQNASLLKVYSKEDQDLLKLVKSYHWMGLVHIPTNGSWQWEDGSI |
| (aa1-71)-CD27 | LSPNLLTIIEMQKGDCALYASSFKGYIENCSTPNTYICMQRTVRRKRSGS |
| endodomain (aa213- | GEGRGSLLTCGDVEENPGPMIHLGHILFLLLLPVAAAQTTPGERSSLPAFY |
| 260) (Construct 10) | PGTSGSCSGCGSLSLPLLAGLVAADAVASLLIVGAVFLCQRRKYRSNKGE |
| (SEQ ID NO: 68) | SPVEPAEPCHYSCPREEEGSTIPIQEDYRKPEPACSP |
| DAP10 (aa1-93)- | MIHLGHILFLLLLPVAAAQTTPGERSSLPAFYPGTSGSCSGCGSLSLPLLA |
| DAP12 endodomain | GLVAADAVASLLIVGAVFLCARPRRSPAQEDGKVYINMPGRGYFLGRLVP |
| (aa62-113)-Furin | RGRGAAEAATRKQRITETESPYQELQGQRSDVYSDLNTQRPYYKRRKRS |
| cleavage-linker-P2A- | GSGATNFSLLKQAGDVEENPGPMGWIRGRRSRHSWEMSEFHNYNLDLK |
| NKG2D (aa1-216)- | KSDFSTRWQKQRCPVVKSKCRENASPFFFCCFIAVAMGIRFIIMVAIWSA |
| Furin cleavage- | VFLNSLFNQEVQIPLTESYCGPCPKNWICYKNNCYQFFDESKNWYESQA |
| linker-T2A-CD8α | SCMSQNASLLKVYSKEDQDLLKLVKSYHWMGLVHIPTNGSWQWEDGSI |
| leader (aa1-21)- | LSPNLLTIIEMQKGDCALYASSFKGYIENCSTPNTYICMQRTVRRKRSGS |
| FLAG-DAP10 (aa19- | GEGRGSLLTCGDVEENPGPMALPVTALLLPLALLLHAARPDYKDDDDKQT |
| 71)-CD27 | TPGERSSLPAFYPGTSGSCSGCGSLSLPLLAGLVAADAVASLLIVGAVFLC |
| endodomain (aa213- | QRRKYRSNKGESPVEPAEPCHYSCPREEEGSTIPIQEDYRKPEPACSP |
| 260) (Construct 11) | |
| (SEQ ID NO: 69) | |

### Genetic Modification

“Genetic modification”, as used herein, will be understood to refer to the introduction of exogenous polynucleotide(s) into a cell or organism to enable the expression of the protein(s) encoded by the exogenous polynucleotide(s) in the cell.

Genetic modification may comprise or consist of transfection or transduction. As used herein, the term “transfection” encompasses a wide variety of techniques commonly used for the introduction of exogenous DNA into a cell, e.g., electroporation, calcium-phosphate precipitation, DEAE-dextran transfection and the like.

The exogenous polynucleotide may be DNA or RNA. The polynucleotide may encode the polypeptide sequence of any one or more of SEQ ID NOs: 60-69, 87, 90, and 102.

In some embodiments, the polynucleotide has at least about 80%, at least about 85%, at least about 90%, at least about 95%, at least about 97% or at least about 99% sequence identity with a polynucleotide sequence encoding any of SEQ ID NOS: 60-69, 87, 90, and 102. Sequence identity is typically measured along the full length of the reference sequence.

The polynucleotide may have at least about 80%, at least about 85%, at least about 90%, at least about 95%, at least about 97% or at least about 99% sequence identity with any one of SEQ ID NOs: 70-79, 88, 91, and 103. In some embodiments, the polynucleotide comprises or consists of the nucleotide sequence of any one of SEQ ID NOs: 70-79, 88, 91, and 103.

Unless specifically limited herein, the term “polynucleotide” encompasses polynucleotides containing known analogues of natural nucleotides that have similar properties as the reference polynucleotide and are metabolized in a manner similar to naturally occurring nucleotides. Examples of such analogs include, without limitation, phosphorothioates, phosphoramidates, methyl phosphonates, chiral-methyl phosphorates, 2-O-methyl ribonucleotides, peptide-nucleic acids (PNAs). Unless otherwise indicated, a particular polynucleotide sequence also implicitly encompasses conservatively modified variants thereof (e.g., degenerate codon substitutions) and complementary sequences, as well as the sequence explicitly indicated. Specifically, as detailed below, degenerate codon substitutions may be achieved by generating sequences in which the third position of one or more selected (or all) codons is substituted with mixed-base and/or deoxyinosine residues (Batzer et al., 1991, Nucleic Acid Res. 19:5081; Ohtsuka et al., 1985, J. Biol. Chem. 260:2605-2608; and Rossolini et al., 1994, Mol. Cell. Probes 8:91-98).

The polynucleotide sequences can be produced by de novo solid-phase DNA synthesis or by PCR mutagenesis of an existing sequence (e.g., sequences as described in the Examples below). Direct chemical synthesis of nucleic acids can be accomplished by methods known in the art, such as the phosphotriester method of Narang et al., 1979, Meth. Enzymol. 68:90; the phosphodiester method of Brown et al., 1979, Meth. Enzymol. 68:109; the diethylphosphoramidite method of Beaucage et al., 1981, Tetra. Lett., 22:1859; and the solid support method of U.S. Pat. No. 4,458,066. Introducing mutations to a polynucleotide sequence by PCR can be performed as described in, e.g., PCR Technology: Principles and Applications for DNA Amplification, H. A. Erlich (Ed.), Freeman Press, NY, N.Y., 1992; PCR Protocols: A Guide to Methods and Applications, Innis et al. (Ed.), Academic Press, San Diego, Calif, 1990; Mattila et al., 1991, Nucleic Acids Res. 19:967; and Eckert et al., 1991, PCR Methods and Applications 1:17.

Preferably, genetic modification comprises or consists of transduction. Typically, transduction involves the introduction of exogenous polynucleotide(s) into a cell by a virus or viral vector. Thus, genetic modification may comprise the delivery of a vector encoding the NKG2D polypeptide (and optionally other polypeptides disclosed), into the cell.

Various vectors can be employed to express polynucleotides encoding the polypeptide(s) of the invention. Both viral-based and non-viral expression vectors can be used to produce the polypeptide(s) in the immune cell. Non-viral vectors and systems include plasmids, episomal vectors, typically with an expression cassette for expressing a protein or RNA, and human artificial chromosomes (see, e.g., Harrington et al., 1997, Nat Genet. 15:345). For example, non-viral vectors useful for expression of the polynucleotides and polypeptides of the invention in mammalian (e.g., human) cells include pThioHis A, B and C, pcDNA3.1/His, pEBVHis A, B and C, (Invitrogen, San Diego, Calif.), MPS V vectors, and numerous other vectors known in the art for expressing other proteins. Useful viral vectors include vectors based on retroviruses, adenoviruses, adeno-associated viruses, herpes viruses, vectors based on SV40, papilloma virus, HBP Epstein Barr virus, vaccinia virus vectors and Semliki Forest virus (SFV). See, Brent et al., supra; Smith, 1995, Annu. Rev. Microbiol. 49:807; and Rosenfeld et al., 1992, Cell 68:143. In particular, retroviral, lentiviral, adenoviral or adeno-associated viral vectors are commonly used for expression in T-cells. Examples of such vectors include the SFG retroviral expression vector (see Riviere et al., 1995, Proc. Natl. Acad. Sci. (USA) 92:6733-6737).

Preferably, genetic modification comprises the delivery of a viral vector encoding the NKG2D polypeptide (and optionally other polypeptides disclosed), into the cell. More preferably, the viral vector is a lentiviral or retroviral vector. The lentiviral vector may comprise a self-inactivating lentiviral vector (so-called SIN vectors). The retroviral vector may comprise an SFG retroviral expression vector.

The vector may comprise expression control sequences, such as an origin of replication, a promoter, and an enhancer (see, e.g., Queen, et al., 1986, Immunol. Rev. 89:49-68), and necessary processing information sites, such as ribosome binding sites, RNA splice sites, polyadenylation sites, and transcriptional terminator sequences. These vectors usually contain promoters derived from mammalian genes or from mammalian viruses. Suitable promoters may be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPS V promoter, the tetracycline-inducible CMV promoter (such as the human immediate-early CMV promoter), the constitutive CMV promoter, the EF1 alpha promoter, the phosphoglycerate kinase (PGK) promoter and promoter-enhancer combinations known in the art.

In some embodiments, the vector comprises a polynucleotide sequence having at least about 80%, at least about 85%, at least about 90%, at least about 95%, at least about 97% or at least about 99% sequence identity with a polynucleotide encoding any of SEQ ID NOS: 60-69, 87, 90, and 102. In some embodiments, the vector comprises a polynucleotide sequence encoding one or more of SEQ ID NOs: 60-69, 87, 90, and 102. In some embodiments, the vector comprises the polynucleotide sequence of any one of SEQ ID NOS: 70-79, 88, 91, and 103.

### Immune Cell

As the skilled person will appreciate, an immune cell is a cell of the immune system. In other words, an immune cell will be understood to be a cell which can respond to infectious organisms. Any such immune cell is suitable for the present invention.

Exemplary immune cells may include, but not necessarily be limited to neutrophils, eosinophils, basophils, mast cells, monocytes, macrophages, dendritic cells, natural killer cells, and lymphocytes.

As the skilled person will appreciate, lymphocytes include B cells and T-cells.

In some embodiments, the immune cell is a neutrophil, macrophage, dendritic cell, natural killer cell or lymphocyte.

In some embodiments, the immune cell is a B cell, T-cell or Natural Killer cell. Preferably, the immune cell is a T-cell or a Natural Killer (NK) cell.

More preferably, the immune cell is a T-cell. T-cells include CD4 T-cells, CD8 T-cells and NKT-cells.

In some embodiments, the T-cell is a CD4⁺ or CD8⁺ T-cell. In some embodiments, the immune cell is a CD4⁺ T-cell. In other embodiments, the immune cell is a CD8⁺ T-cell.

In some embodiments, the immune cell is an αβ or a γδ T-cell. In some embodiments, the immune cell is a αβ T-cell. In other embodiments, the immune cell is a γδ T-cell.

In some embodiments, the immune cell is a peripheral blood mononuclear cell (PBMC). Following activation and genetic modification, as described above, the PBMC may differentiate into another immune cell described above. For example, after activation and genetic modification, the PBMC may have differentiated into a T-cell.

### Exemplary Methods of the Invention

Preferably, the method is a method of making an immunoresponsive T or NK cell and comprises:
(i) activating a PBMC with anti-CD3 and anti-CD28 antibodies, or fragments thereof, conjugated to a nanomatrix; and
(ii) genetically modifying the PBMC to express the NKG2D polypeptide, a DAP10/DAP12 fusion polypeptide and a CXCR2 polypeptide, wherein genetic modification comprises transduction of the PBMC with a retroviral vector encoding the NKG2D polypeptide, a DAP10/DAP12 fusion polypeptide and a CXCR2 polypeptide.

In some embodiments, the method is a method of making an immunoresponsive T or NK cell and comprises:
(i) activating a PBMC with anti-CD3 and anti-CD28 antibodies, or fragments thereof, conjugated to a nanomatrix such that the PBMC differentiates into a T or NK cell; and
(ii) genetically modifying the T or NK cell to express the NKG2D polypeptide, a DAP10/DAP12 fusion polypeptide and a CXCR2 polypeptide, wherein genetic modification comprises transduction of the T or NK cell with a retroviral vector encoding the NKG2D polypeptide, a DAP10/DAP12 fusion polypeptide and a CXCR2 polypeptide.

In some embodiments, the method is a method of making an immunoresponsive T or NK cell and comprises:
(i) activating a PBMC with anti-CD3 and anti-CD28 antibodies, or fragments thereof, conjugated to a nanomatrix such that the PBMC differentiates into a T or NK cell, wherein the nanomatrix comprises iron oxide crystals embedded into a biocompatible polysaccharide matrix with a diameter of about 100 nm; and
(ii) genetically modifying the T or NK cell to express the NKG2D polypeptide, a DAP10/DAP12 fusion polypeptide and a CXCR2 polypeptide, wherein genetic modification comprises transduction of the T or NK cell with a retroviral vector encoding the NKG2D polypeptide, a DAP10/DAP12 fusion polypeptide and a CXCR2 polypeptide.

In some embodiments, the method is a method of making an immunoresponsive T or NK cell and comprises:
(i) activating a PBMC with anti-CD3 and anti-CD28 antibodies, or fragments thereof, conjugated to a nanomatrix such that the PBMC differentiates into a T or NK cell, wherein the nanomatrix is colloidal and comprises iron oxide crystals embedded into a biocompatible polysaccharide matrix with a diameter of about 100 nm; and
(ii) genetically modifying the T or NK cell to express the NKG2D polypeptide, a DAP10/DAP12 fusion polypeptide and a CXCR2 polypeptide, wherein genetic modification comprises transduction of the T or NK cell with a retroviral vector encoding the NKG2D polypeptide, a DAP10/DAP12 fusion polypeptide and a CXCR2 polypeptide.

According to another aspect there is provided a method of making an immunoresponsive cell, wherein the method comprises:
(a) activating an immune cell, wherein activating does not comprise phorbol 12-myristate 13-acetate (PMA), phytohaemagglutinin (PHA) or beads; and
(b) genetically modifying the immune cell to express the NKG2D polypeptide.

The order of activation and genetic modification may be as defined above. Likewise, the genetic modification and immune cell may be as defined above. For example, genetic modification may comprise transduction. The immune cell may be a PBMC. In some embodiments, the immune cell is a T-cell or neutrophil.

Genetic modification may comprise genetically modifying an immune cell to express an NKG2D polypeptide and a DAP10/DAP12 fusion polypeptide. In some embodiments genetic modification comprises genetically modifying an immune cell to express an NKG2D polypeptide and a CXCR2 polypeptide. In some embodiments genetic modification comprises genetically modifying an immune cell to express an NKG2D polypeptide, a DAP10/DAP12 fusion polypeptide and a CXCR2 polypeptide.

### Immunoresponsive Cells

The invention also provides an immunoresponsive cell obtainable by any of the methods of the invention.

According to another aspect, there is also provided an immunoresponsive cell genetically modified to express an NKG2D polypeptide; wherein the immunoresponsive cell has been activated with anti-CD3 and anti-CD28 antibodies, or fragments thereof, conjugated to a nanomatrix.

In some embodiments the immunoresponsive cell is genetically modified to express an NKG2D polypeptide and a fusion polypeptide comprising (i) a DNAX-activating 10 (DAP10) polypeptide, or a functional variant thereof and (ii) a DNAX-activating protein 12 (DAP12) polypeptide, or a functional variant thereof. The NKG2D polypeptide may be as defined above. The fusion polypeptide may be as defined above.

In some embodiments, the DAP10 polypeptide is a functional variant of DAP10 comprising an amino acid sequence having at least about 80%, at least about 85%, at least about 90%, at least about 95%, at least about 97%, at least about 98% or at least about 99% sequence identity to the DAP10 polypeptide of SEQ ID NO: 1. In some embodiments, the DAP10 polypeptide comprises or consists of an amino acid sequence having the sequence of SEQ ID NO: 1.

In some embodiments, the DAP10 polypeptide is a truncated version of DAP10 which comprises or consists of amino acids 19-93, 19-69, 1-71, 19-71, 19-48, 49-69, 49-93, or 70-93 of SEQ ID NO: 1.

In some embodiments, the DAP10 polypeptide comprises or consists of any one of SEQ ID NOs: 1-8.

In some embodiments, the DAP12 polypeptide is a functional variant of DAP12 comprising an amino acid sequence having at least about 80%, at least about 85%, at least about 90%, at least about 95%, at least about 97%, at least about 98% or at least about 99% sequence identity to the DAP12 polypeptide having the sequence of SEQ ID NO: 9. In some embodiments, the DAP12 polypeptide comprises or consists of an amino acid sequence having the sequence of SEQ ID NO: 9.

In some embodiments, the DAP12 polypeptide is a truncated version of DAP12 which comprises or consists of amino acids 22-113, 62-113, 22-61, or 41-61 of SEQ ID NO: 9.

In some embodiments, the DAP12 polypeptide comprises or consists of any one of SEQ ID NOs: 9-13.

In some embodiments the NKG2D polypeptide is a human polypeptide. In some embodiments, the NKG2D polypeptide is a functional variant of NKG2D. The functional variant of NKG2D may comprise an amino acid sequence having at least about 80%, at least about 85%, at least about 90%, at least about 95%, at least about 97%, at least about 98% or at least about 99% sequence identity to the NKG2D polypeptide having the sequence of SEQ ID NO: 14. In some embodiments, the NKG2D polypeptide comprises or consists of SEQ ID NO: 14.

The functional variant of NKG2D may comprise or consist of a truncated version of NKG2D. In some embodiments, the NKG2D polypeptide may be a truncated version of NKG2D comprising only amino acids 73-216 of SEQ ID NO: 14 (i.e., the extracellular domain. Such a sequence is referred to as SEQ ID NO: 15 herein. Other truncated versions may comprise amino acids 82-216 of SEQ ID NO: 14, such a sequence comprising part of the extracellular domain of NKG2D and referred to herein as SEQ ID NO: 16. A further truncated version comprises amino acids 52-216 of SEQ ID NO: 14 (i.e. the transmembrane and extracellular domains), referred to as SEQ ID NO: 17 herein.

In some embodiments, the functional variant of the NKG2D polypeptide is a chimeric NKG2D polypeptide. The chimeric NKG2D polypeptide may be a human-murine chimeric polypeptide.

In some embodiments, the chimeric NKG2D polypeptide comprises from N terminus to C terminus the murine NKG2D transmembrane domain or a variant thereof and a human NKG2D extracellular domain or a variant thereof.

In some embodiments, the immune cell is genetically modified to further express a CXCR2 polypeptide. Thus, in some embodiments, the immunoresponsive cell is genetically modified to express an NKG2D polypeptide and a CXCR2 polypeptide. In some embodiments the immunoresponsive cell is genetically modified to express an NKG2D polypeptide, a CXCR2 polypeptide and a DAP10/DAP12 fusion polypeptide as defined above.

The CXCR2 polypeptide may be as defined above. For example, the CXCR2 polypeptide may be a mammalian polypeptide. The CXCR2 polypeptide may be a human polypeptide.

In some embodiments, the CXCR2 polypeptide comprises a functional variant of the wildtype CXCR2 polypeptide. Thus, the CXCR2 polypeptide may be a functional variant of the CXCR2 polypeptide of SEQ ID NO: 87. The functional variant of the CXCR2 polypeptide may comprise an amino acid sequence having at least about 80%, at least about 85%, at least about 90%, at least about 95%, at least about 97%, at least about 98%, or at least about 99% sequence identity to the CXCR2 polypeptide having the sequence of SEQ ID NO: 87. In some embodiments, the CXCR2 polypeptide comprises or consists of amino acid sequence of SEQ ID NO: 87.

The immunoresponsive cell is an immune cell. The immunoresponsive cell may comprise a neutrophil, an eosinophil, a basophil, mast-cell, monocyte, macrophage, dendritic cell, natural killer cell or lymphocyte. In some embodiments, the immune cell is a neutrophil, macrophage, dendritic cell, natural killer cell or lymphocyte.

Preferably, the immunoresponsive cell is a lymphocyte or natural killer (NK) cell. Thus, the immunoresponsive cell may be a T-cell, B-cell or NK cell. Preferably, the immunoresponsive cell is a T-cell or a Natural Killer (NK) cell. In some embodiments, the immunoresponsive cell is a T-cell. In some embodiments, the T-cell is a CD4⁺ or CD8⁺ T-cell. In some embodiments, the immunoresponsive cell is a CD4⁺ T-cell. In other embodiments, the immunoresponsive cell is a CD8⁺ T-cell.

In some embodiments, the immunoresponsive cell is an αβ or a γδ T-cell. In some embodiments, the immunoresponsive cell is a αβ T-cell. In other embodiments, the immunoresponsive cell is a γδ T-cell.

In some embodiments, the immunoresponsive cell is a peripheral blood mononuclear cell (PBMC). Alternatively, the immunoresponsive cell may be derived from a PBMC. In the context of the present invention, “derived from a PBMC” will be understood to refer to a cell which has differentiated from a PBMC.

Preferably, the immunoresponsive cell comprises a population of immunoresponsive cells. More preferably, the immunoresponsive cell comprises a population T or NK cells. Most preferably, the immunoresponsive cell comprises a population of T-cells. The population of T-cells may comprise CD4⁺ T-cells. The population of T-cells may comprise CD8⁺ T-cells. In some embodiments, the population of T-cells comprises CD4⁺ T-cells and CD8⁺ T-cells.

The inventors have unexpectedly found that immunoresponsive cells activated with anti-CD3 and anti-CD28 antibodies, or fragments thereof, conjugated to the nanomatrix of the invention have greater fold expansion and thus population numbers than immunoresponsive cells activated using other activation stimuli.

Thus, in some embodiments the population comprises at least about 1×10⁵ immunoresponsive cells, at least about 5×10⁵ immunoresponsive cells, at least about 1×10⁶ immunoresponsive cells, at least about 5×10⁶ immunoresponsive cells, at least about 1×10⁷ immunoresponsive cells, at least about 5×10⁷ immunoresponsive cells, at least about 1×10⁸ immunoresponsive cells, at least about 5×10⁸ immunoresponsive cells, at least about 1×10⁹ immunoresponsive cells, at least about 5×10⁹ immunoresponsive cells, at least about 1×10¹⁰ immunoresponsive cells, at least about 5×10¹⁰ immunoresponsive cells or at least about 1×10¹¹ immunoresponsive cells.

In some embodiments the population comprises at least about 1×10⁶ immunoresponsive cells.

In some embodiments, the population of immunoresponsive cells has an expansion rate of at least about five-fold every ten days. In some embodiments, the population of immunoresponsive cells has an expansion rate of at least about ten-fold every ten days. In some embodiments, the population of immunoresponsive cells has an expansion rate of at least about 20-fold every ten days. In some embodiments, the population of immunoresponsive cells has an expansion rate of at least about 30-fold every ten days. In some embodiments, the population of immunoresponsive cells has an expansion rate of at least about 40-fold every ten days. In some embodiments, the population of immunoresponsive cells has an expansion rate of at least about 50-fold every ten days. In some embodiments, the population of immunoresponsive cells has an expansion rate of at least about 60-fold every ten days. In some embodiments, the population of immunoresponsive cells has an expansion rate of at least about 70-fold every ten days. In some embodiments, the population of immunoresponsive cells has an expansion rate of at least about 80-fold every ten days. In some embodiments, the population of immunoresponsive cells has an expansion rate of at least about 90-fold every ten days. In some embodiments, the population of immunoresponsive cells has an expansion rate of at least about 100-fold every ten days.

Preferably, the immunoresponsive cell comprises a population of T-cells having an expansion rate of at least about five-fold every ten days. More preferably, the immunoresponsive cell comprises a population of T-cells having an expansion rate of at least about ten-fold every ten days. Most preferably, the immunoresponsive cell comprises a population of T-cells having an expansion rate of at least about 20-fold every ten days.

The inventors have also found that activation of T-cells with anti-CD3 and anti-CD28 antibodies, or fragments thereof, conjugated to the nanomatrix of the invention leads to an unexpected increase in CD4⁺ T-cells, relative to T-cells activated with other stimuli. Thus, in some embodiments, the population of T-cells comprises a CD4:CD8 ratio of at least about 1:1. In some embodiments, the population of T-cells comprises a CD4:CD8 ratio of at least about 2:1. Alternatively, the population of T-cells may comprise a CD4:CD8 ratio of at least about 3:1, optionally of about 4:1.

### Pharmaceutical Compositions

Also provided by the invention is a pharmaceutical composition comprising the immunoresponsive cell obtainable by either of the methods disclosed herein or the immunoresponsive cell of the above aspect and a pharmaceutically or physiologically acceptable diluent and/or carrier.

As used herein, the term “pharmaceutical composition” refers to the combination of an active ingredient with a carrier, inert or active, making the composition especially suitable for therapeutic or diagnostic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

The carrier is generally selected to be suitable for the intended mode of administration and can include agents for modifying, maintaining, or preserving, for example, the pH, osmolarity, viscosity, clarity, colour, isotonicity, odour, sterility, stability, rate of dissolution or release, adsorption, or penetration of the composition. Typically, these carriers include aqueous or alcoholic/aqueous solutions, emulsions or suspensions, including saline and/or buffered media.

Suitable agents for inclusion in the pharmaceutical compositions include, but are not limited to, amino acids (such as glycine, glutamine, asparagine, arginine, or lysine), antimicrobials, antioxidants (such as ascorbic acid, sodium sulphite, or sodium hydrogen-sulphite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as free serum albumin, gelatin, or immunoglobulins), colouring, flavouring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as Polysorbate 20 or Polysorbate 80; Triton; tromethamine; lecithin; cholesterol or tyloxapal), stability enhancing agents (such as sucrose or sorbitol), tonicity enhancing agents (such as alkali metal halides, such as sodium or potassium chloride, or mannitol sorbitol), delivery vehicles, diluents, excipients and/or pharmaceutical adjuvants.

Parenteral vehicles include sodium chloride solution, Ringer's dextrose, dextrose and sodium chloride and lactated Ringer's. Suitable physiologically-acceptable thickeners such as carboxymethylcellulose, polyvinylpyrrolidone, gelatin and alginates may be included. Intravenous vehicles include fluid and nutrient replenishers and electrolyte replenishers, such as those based on Ringer's dextrose. In some cases one might include agents to adjust tonicity of the composition, for example, sugars, polyalcohols such as mannitol, sorbitol, or sodium chloride in a pharmaceutical composition. For example, in many cases it is desirable that the composition is substantially isotonic. Preservatives and other additives, such as antimicrobials, antioxidants, chelating agents and inert gases, may also be present. The precise formulation will depend on the route of administration. Additional relevant principle, methods and components for pharmaceutical formulations are well known (see, e.g., Allen, Loyd V. Ed, (2012) Remington's Pharmaceutical Sciences, 22nd Edition).

A pharmaceutical composition of the present invention can be administered by one or more routes of administration using one or more of a variety of methods known in the art. As will be appreciated by the skilled person, the route and/or mode of administration will vary depending upon the desired results. Routes of administration for pharmaceutical compositions of the invention include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intra-sternal injection and infusion. In one embodiment, the pharmaceutical composition is administered intratumourally. When parenteral administration is contemplated, the pharmaceutical compositions are usually in the form of a sterile, pyrogen-free, parenterally acceptable composition. A particularly suitable vehicle for parenteral injection is a sterile, isotonic solution, properly preserved. The pharmaceutical composition can be in the form of a lyophilizate, such as a lyophilized cake.

Alternatively, the pharmaceutical composition described herein can be administered by a non-parenteral route, such as a topical, epidermal or mucosal route of administration, for example, intranasally, orally, vaginally, rectally, sublingually or topically.

In some embodiments, the pharmaceutical composition is for subcutaneous administration. Suitable formulation components and methods for subcutaneous administration of polypeptide therapeutics (e.g., antibodies, fusion polypeptides and the like) are known in the art, see, for example, US2011/0044977, U.S. Pat. Nos. 8,465,739 and 8,476,239. Typically, the pharmaceutical compositions for subcutaneous administration contain suitable stabilizers (e.g, amino acids, such as methionine, and or saccharides such as sucrose), buffering agents and tonicifying agents.

Preferably, in cell therapy, the pharmaceutical composition comprising the immunoresponsive cell is administered to the subject by intravenous infusion.

Administration of the pharmaceutically useful composition of the present invention is preferably in a “therapeutically effective amount” or “prophylactically effective amount”, this being sufficient to show benefit to the individual. The actual amount administered, and rate and time-course of administration, will depend on the nature and severity of protein aggregation disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A pharmaceutical composition can be administered alone or in combination with other treatments, either simultaneously or sequentially dependent upon the condition to be treated.

As used herein, the terms “administration” and “administering” refer to the act of giving a drug, prodrug, or other agent, or therapeutic treatment (e.g., peptide) to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal or lingual), ear, rectal, vaginal, by injection (e.g., intravenously, subcutaneously, intratumourally, intraperitoneally, etc.) and the like.

### Kits

The invention also provides a kit comprising the immunoresponsive cell obtainable by the methods disclosed herein, the immunoresponsive cell of the invention or the pharmaceutical composition of the invention.

The kit may further comprise instructions for use. In some embodiments, the immunoresponsive cells and/or the pharmaceutical composition is provided in an aqueous solution in the kit, optionally buffered solution and/or at a temperature of at least −20° C.

Components or embodiments described herein for the system may be provided in a kit. For example, any of the vectors, as well as the mammalian cells, related buffers, media, triggering agents, or other components related to cell culture and virion production can be provided, with optional components frozen and packaged as a kit, alone or along with separate containers of any of the other agents and optional instructions for use. In some embodiments, the kit may comprise culture vessels, vials, tubes, or the like.

### Methods of Treatment

Also provided is a method of treating or preventing cancer in a subject, wherein the method comprises administering to the subject the immunoresponsive cell or the pharmaceutical composition of the invention.

As used herein, the term “treatment” means an approach to obtaining a beneficial or intended clinical result. The beneficial or intended clinical result can include alleviation of symptoms, a reduction in the severity of the disease, inhibiting an underlying cause of a disease or condition, steadying diseases in a non-advanced state, delaying the progress of a disease, and/or improvement or alleviation of disease conditions.

The method typically comprises administering a therapeutically effective amount or a prophylactically effective amount of the immunoresponsive cell or the pharmaceutical composition of the invention. A therapeutically effective amount is an amount which ameliorates one or more symptoms, such as all the symptoms, of the disease and/or abolishes one or more symptoms, such as all the symptoms, of the disease. The therapeutically effective amount preferably cures the disease. A prophylactically effective amount is an amount which prevents the onset of the disease and/or prevents the onset of one or more symptoms, such as all the symptoms, of the disease. The prophylactically effective amount preferably prevents the subject from developing the disease. Suitable amounts are discussed in more detail below.

The immunoresponsive cells or pharmaceutical composition of the invention may be administered to a subject that displays symptoms of disease. The immunoresponsive cells or pharmaceutical composition of the invention may be administered to a subject that is asymptomatic, i.e. does not display symptoms of disease. The immunoresponsive cells or pharmaceutical composition of the invention may be administered when the subject's disease status is unknown or the patient is expected not to have a disease. The immunoresponsive cells or pharmaceutical composition of the invention may be administered to a subject that is predisposed, such as genetically predisposed, to developing the disease.

As used herein, the term “subject” broadly refers to any animal, including but not limited to, human and non-human animals (e.g., dogs, cats, cows, horses, sheep, pigs, poultry, fish, crustaceans, etc.).

The subject may be a mammal. Optionally, the subject is a human, horse, dog or cat. In some embodiments, the subject is human. Alternatively, the subject may be a horse.

The cancer may include, but not necessarily be limited to, a solid tumour cancer, a soft tissue tumour, a metastatic lesion, and a haematological cancer. For example, the cancer can be liver cancer, lung cancer, breast cancer, prostate cancer, lymphoid cancer, colon cancer, renal cancer, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumours of childhood, lymphocytic lymphoma, cancer of the bladder, cancer of the kidney or ureter, carcinoma of the renal pelvis, neoplasm of the central nervous system (CNS), primary CNS lymphoma, tumour angiogenesis, spinal axis tumour, brain stem glioma, pituitary adenoma, Kaposi's sarcoma, epidermoid cancer, squamous cell cancer, T-cell lymphoma, myelodysplastic syndrome (MDS), chronic myelogenous leukaemia-chronic phase (CMLCP), diffuse large B-cell lymphoma (DLBCL), cutaneous T-cell lymphoma (CTCL), peripheral T-cell lymphoma (PTCL), hepatocellular carcinoma (HCC), gastrointestinal stromal tumours (GIST), non-small cell lung carcinoma (NSCLC), squamous cell carcinoma of the head and neck (SCCHN), environmentally induced cancers including those induced by asbestos, and combinations of said cancers. In particular, the cancer can be breast cancer, such as an estrogen receptor-positive (ER pos) breast cancer and/or a metastatic form of breast cancer.

The cancer may be a solid tumour cancer.

In some embodiments, the cancer is selected from the group consisting of cancer of the head and/or neck, ovarian cancer, malignant mesothelioma, breast cancer, pancreatic cancer, colorectal cancer, lung cancer, gastric cancer, bladder cancer, prostate cancer, oesophageal cancer, endometrial cancer, hepatobiliary cancer, chronic or acute leukaemia including acute myeloid leukaemia, duodenal carcinoma, thyroid carcinoma, cancer of the central nervous system or renal cell carcinoma.

In some embodiments, the cancer is selected from ovarian cancer, breast cancer, optionally triple-negative breast cancer, pancreatic cancer, chronic or acute leukaemia including acute myeloid leukaemia, malignant mesothelioma, and combinations of said cancers.

In some embodiments, the cancer is breast cancer. Breast cancer includes, but is not necessarily limited to estrogen receptor-positive (ER pos) breast cancer, estrogen receptor-negative (ER neg) breast cancer, progesterone receptor-positive (PR pos) breast cancer, progesterone receptor-negative (PR neg) breast cancer, HER2-positive (HER2 pos) breast cancer, HER2-negative (HER2 neg) breast cancer, and triple negative (TNBC) breast cancer. TNBC breast cancer is breast cancer wherein the cancer cells test negative (i.e. express no or negligible amounts) for the estrogen receptor, progesterone receptor and HER2. In some embodiments, the breast cancer comprises ER pos, ER neg, PR pos, PR neg, HER2 pos, HER2 neg, and/or combinations thereof.

In some embodiments, the breast cancer comprises triple negative breast cancer (TNBC).

In some embodiments, treatment of the cancer involves targeting of non-tumour cells, such as tumour-associated stromal cells. Example types of such tumour-associated stromal cells include pancreatic stromal cells. Other types of non-tumour cells that may be targeted include macrophages, regulatory T-cells and myeloid-derived suppressor cells. The subject may have been pre-treated with a chemotherapeutic agent.

The administration of immunoresponsive cells or pharmaceutical composition of the invention to the subject may result in a decrease in tumour size of about 10%, about 20%, about 30%, about 40%, about 50%, about 60%, about 70%, about 80%, about 90%, or even about 100%, when compared to an untreated tumour.

In embodiments administering immunoresponsive cells, the number of cells administered to the subject should take into account the route of administration, the disease being treated, the weight of the subject and/or the age of the subject. In general, from about 1×10⁶ to about 1×10¹¹ immunoresponsive cells may be administered to the subject. In some embodiments, from about 1×10⁷ to about 1×10¹⁰ immunoresponsive cells, or from about 1×10⁸ to about 1×10⁹ immunoresponsive cells are administered to the subject.

The invention also provides the immunoresponsive cell or pharmaceutical composition of the invention for use in any of the therapeutic methods described above. Thus, also provided is the immunoresponsive cell or pharmaceutical composition of the invention for use in the treatment or prevention of a disease. Preferably, the disease is cancer. This may otherwise be referred to as for use in therapy. More preferably, the invention provides the immunoresponsive cells or pharmaceutical composition of the invention for use in the treatment or prevention of breast cancer. Most preferably, the invention provides the immunoresponsive cells or pharmaceutical composition of the invention for use in the treatment or prevention of triple negative breast cancer

Also provided is use of the immunoresponsive cell or the pharmaceutical composition of the invention for the manufacture of a medicament for the treatment or prevention of a disease. Preferably, the disease is cancer, as described above. The invention also provides use of the immunoresponsive cell or the pharmaceutical composition of the invention for (i) therapy or (ii) the treatment of cancer. In some embodiments, the cancer is breast cancer. In some embodiments, the breast cancer is triple-negative breast cancer.

All publications, patents and patent applications cited herein, whether supra or infra, are hereby incorporated by reference in their entirety.

As used herein, the words “comprise” and “contain” and variations of the words, for example, “comprising” and “comprises”, mean “including but not limited to”, and do not exclude other components, integers or steps. Moreover, the singular encompasses the plural unless the context otherwise requires: in particular, where the indefinite article is used, the specification is to be understood as contemplating plurality as well as singularity, unless the context requires otherwise.

### Examples

Below are examples of specific embodiments for carrying out the present invention. The examples are offered for illustrative purposes only and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature.

### Materials and Methods

### T-Cell Isolation and Retroviral Transduction

Peripheral blood mononuclear cells (PBMCs) were isolated from blood samples from healthy volunteers through density-mediated centrifugation. T-cells were activated as described below. 1×10⁶ activated PBMC were plated onto a RetroNectin-coated plate that had been pre-treated with 3 mL retroviral supernatant. Each well was subsequently treated with 3 mL fresh viral supernatant and 100 IU/mL IL-2. Retroviral transduction was performed with viral particles produced by stable gibbon ape leukemia virus (GALV)-pseudotyped 293TVec stable packaging cells. Thereafter, T-cells were fed with 100 IU/mL in RPMI1640 media+5% normal human AB serum, with fresh media and IL-2 (100 IU/mL) provided thrice weekly.

### T-Cell Activation

T-cells were activated using paramagnetic beads coated with anti-human CD3 and anti-human CD28 antibodies (1:2 cell:bead ratio), phytohaemagglutinin (PHA) at a concentration of 5 ug/mL or 10 uL TransAct reagent per 1×10⁶ PBMCs.

### Flow Cytometry

T-cell transduction and transfection of 293T-cells was assessed by flow cytometry, making comparison, where indicated, with an appropriate isotype control. To assess the expression of the NKG2D-based constructs, cells were stained with mouse anti-human CD4-FITC, mouse anti-human NKG2D-PE and mouse anti-human CD8-APC and compensated appropriately. Due to high levels of endogenous NKG2D expression in CD8⁺ T-cells, transduction efficiency was compared against NKG2D expression in untransduced CD4⁺ T-cells. Prior to use, the transduction efficiency was normalized between constructs by spiking in the requisite proportion of untransduced T-cells. This ensured that all conditions were identical for the total number of CAR⁺ T-cells and overall T-cell concentration.

To assess T-cell differentiation after multiple rounds of stimulation, T-cells were removed from tumour cell co-cultures and stained with FITC-conjugated anti-human CD45RO, allophycocyanin (APC)-conjugated anti-human CD62L, PE-conjugated anti-human CCR7, FITC-conjugated anti-human CD4, APCCy7-conjugated anti-human CD8a and phycoerythrin cyanine7 (PECy7)-conjugated anti-human CD27 antibodies. After washing in 2 mL ice-cold PBS the cells were re-suspended in 0.5 mL ice-cold PBS and assessed by flow cytometry. Staining efficiency was assessed using T-cells stained with the appropriate isotype and fluorescence minus one (FMO) controls.

### Dose Response Assays

1×10⁴ tumour cells were plated per well (100 μL) of a 96-well plate and incubated at 37° C. and 5% CO₂ overnight. Twenty-four hours later T-cells were added at log² CAR T-cell:tumour cell ratios ranging from 1:1 to 1:64. After 72 hours, the T-cells were removed and 100 μL MTT solution (500 μg/mL) added, before the plates were incubated at 37° C. and 5% CO₂ for approximately 1 hour. Following removal of the MTT solution, the resulting formazan crystals were solubilized in DMSO (100 μL/well) and the absorbance measured at 560 nm. Tumour cell viability was calculated as follows: (Absorbance of monolayer with T-cells/absorbance of monolayer without T-cells)*100.

### Re-Stimulation Assays

1×10⁵ tumour cells were plated in triplicate wells of a 24-well plate and incubated for 24 hours at 37° C. and 5% CO₂. Twenty-four hours later, 1 mL containing 1×10⁵ CAR⁺ T-cells were added per well. After 72 hours, the T-cells were gently removed and the well was washed with 1 mL PBS. Following removal of the PBS, 1 mL of MTT (at a final concentration of 500 μg/mL) was added to each well and the plate incubated at 37° C. and 5% CO₂ for approximately 1 hour. Absorbance was measured in the appropriate wells at 560 nm and tumour cell viability calculated as detailed in the ‘dose response’ section. A re-stimulation was considered successful if the tumour cell viability was measured as less than 50%.

The T-cells that had been removed from the plate were centrifuged at 400×g for 5 minutes and the supernatant removed. The pellet was re-suspended in 3.2 mL R5 media and 1 ml added to each well of fresh tumour monolayer (1×10⁵ tumour cells per well of a 24-well plate) in triplicate. Total T-cell number was assessed by trypan blue exclusion of a small aliquot of the remaining 200 μL.

### In Vivo Studies

### Bioluminescence Imaging

1×10⁵ firefly luciferase (ffLUC)-tagged BxPC3 cells were injected into the intraperitoneal cavity of NSG mice. Twelve days after tumour inoculation, mice (n=5 per group) were treated intraperitoneally with either PBS, 4×10⁶ (N1012⁺ (N1012 (lo)), or 1×10⁷ (N1012 (hi) or NKG2D) CAR⁺ T-cells. Untransduced T-cells were used as controls.

Tumour growth was monitored by BLI, with all data presented as total flux (photons/second) or average total flux (photons/second) per treatment. Mice were monitored closely and weighed three times per week for signs of ill health.

### Tumour Volume

1×10⁵ CFPac-1 or BxPC3 cells were injected subcutaneously in 50 μl of Matrigel (1:1 PBS) into the left flank of NSG mice. Twenty-nine days after inoculation with CFPac-1 cells or fourteen days after inoculation with BxPC3 cells, mice were treated intravenously with either PBS or 1×10⁷ CAR T-cells (N1012_CXCR2, N1012, NKG2D, CYAD-01 replica, untransduced (UT)).

Tumour growth was measured weekly by caliper measurements and presented as tumour volume (mm³).

### Example 1: Evaluating Anti-Tumour Efficacy of N1012 CXCR2 T-Cells In Vivo in Mouse Models of Pancreatic Cancer

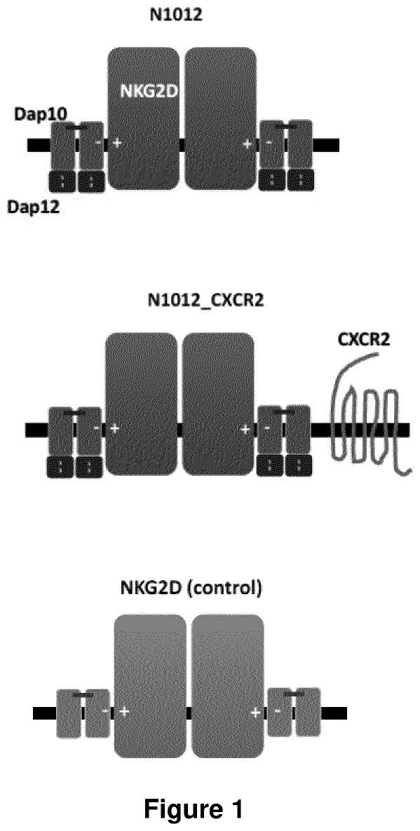

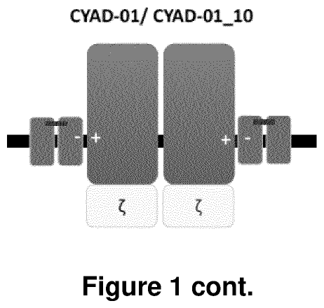

T-cells expressing a CAR construct (e.g., N1012, N1012_CXCR2, CYAD-01_10) were generated by isolating peripheral blood mononuclear cells (PBMCs), activating the T-cells, and transducing the T-cells with virus containing a polynucleotide encoding the CAR (FIG. 1).

Briefly, to generate virus, 1.65×10⁶ HEK293T-cells were seeded in a 10 cm² tissue culture dish in 10 mL of IMDM media containing 10% FBS and 2 mM L-glutamine (110 media) and incubated for 24 hours at 37° C. at 5% CO₂. The following day, transfection mix was generated for each CAR construct according to the protocol in Table 3. The HEK293T-cells were separately transfected with the following plasmids: N1012 (encoding DAP10/12 fusion protein and human NKG2D receptor (SEQ ID NO: 74)), N1012_CXCR2 (encoding DAP10/12 fusion protein, human NKG2D receptor, and CXCR2 (SEQ ID NO: 91)), CYAD-01 replica (encoding NKG2D receptor fused to CD3Z (SEQ ID NO: 93)), and CYAD-01_10 (encoding CYAD-01 replica and DAP10 (SEQ ID NO: 94).

TABLE 3
Transfection protocol for CAR constructs
(volumes per 10 cm² dish)

| Reagent | Volume/Amount | | Incubation Time |
|---|---|---|---|
| Serum-free media | 470 | μL | Incubate for 5 minutes at |
| Genejuice | 30 | μL | room temperature. This is |
| | | | Mix A |
| PeqPam-3 | 4.6875 | μg | Add all three plasmids to |
| RDF | 3.125 | μg | Mix A, gently mix and then |
| SFG vector | 4.6875 | μg | incubate for 15 minutes at |
| containing CAR | | | room temperature. This is |
| (e.g. N1012_CXCR2) | | | now Mix B |

Upon completion of the 15-minute incubation, Mix B was added dropwise to the HEK293T-cells and gently swirled. The cells were then placed back in the incubator. Supernatant was harvested 48 hours after transfection, collected into pre-chilled 50 mL Falcon tubes, and stored at 4° C.

HEK293T-cells were fed with 10 ml of fresh 110 media and returned to the incubator. After an additional 24 hours, the supernatant was harvested a second time from the HEK293T-cells and combined with the supernatant harvested 48 hours after transfection. The combined supernatant was aliquoted into pre-labelled tubes, snap frozen, and stored at −80° C.

PBMCs were isolated using standard Ficoll Paque-mediated density centrifugation. Once re-suspended at a concentration of 1×10⁶ cells/mL in RPMI+5% normal human AB serum and 2 mM L-glutamine (‘R5’ media), the T-cells were activated. Forty-eight hours after activation, 1×10⁶ T-cells were plated onto RetroNectin-coated non-tissue culture treated plates and mixed with 3 mL viral supernatant harvested from transiently transfected HEK 293T-cells. T-cells were fed with 100 IU/mL IL-2 in RPMI1640 media+5% normal human AB serum, with fresh media and IL-2 (100 IU/mL) provided thrice weekly.

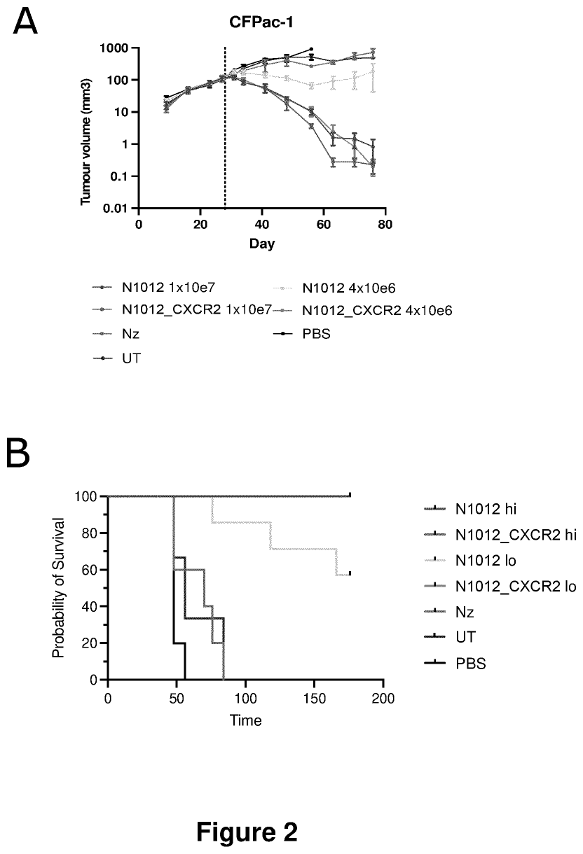

In order to evaluate efficacy of high and low doses of CAR T-cells on pancreatic tumours in vivo, CAR T-cells were injected into mice bearing CFPac-1 tumour cells. Briefly, 1×10⁵ CFPac-1 cells were injected into the intraperitoneal cavity of NSG mice. Twenty-eight days after tumour inoculation, mice were treated with either PBS [n=5], a high dose of 10×10⁶ CAR T-cells (N1012 [n=7], N1012_CXCR2 [n=7], or a low dose of 4×10⁶ CAR T-cells (N1012 [n=6], N1012_CXCR2 [n=7]). Results shown in FIG. 2A demonstrate that efficacy of treatment with a low dose of N1012_CXCR2 T-cells tracks treatment with a high dose of N1012 T-cells. FIG. 2B shows that the probability of survival was highest for N1012_CXCR2 treated mice, particularly those administered the high dose of N1012_CXCR2 cells.

### Example 2: Anti-Tumour Efficacy of N1012 CXCR2 T-Cells Against Breast Cancer Cells

To confirm the efficacy of N1012_CXCR2 T-cells against another cancer model, the restimulation and cytotoxic capacity of N1012_CXCR2 T-cells against the triple negative breast cancer cell lines MDA-MB-468 and MDA-MB-231 was compared to N1012 T-cells.

Briefly, N1012 or N1012_CXCR2 T-cells were co-cultured with fresh monolayer twice weekly until monolayer destruction was not observed. To achieve this, 1×10⁵ MDA-MB-468 or MDA-MB-231 cells were plated in triplicate wells of a 24-well plate and incubated for 24 hours at 37° C. and 5% CO₂. NKG2D⁺ T-cells were used as a control. Twenty-four hours later, 1×10⁵ CAR⁺ T-cells were added per well at a final concentration of 1×10⁵ CAR⁺/mL. After 72 hours, the T-cells were gently removed and centrifuged at 400×g for 5 minutes and the supernatant removed. The pellet was re-suspended in 3.2 mL R5 media and 1 mL added to each well of fresh tumour monolayer in triplicate. Total T-cell number was assessed by trypan blue exclusion of a small aliquot of the remaining 200 μL.

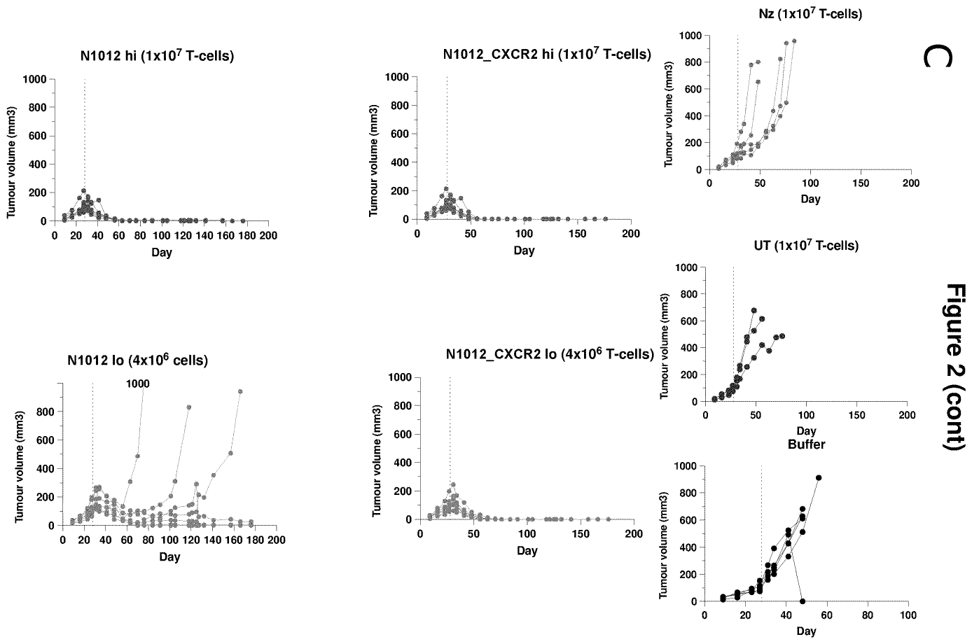

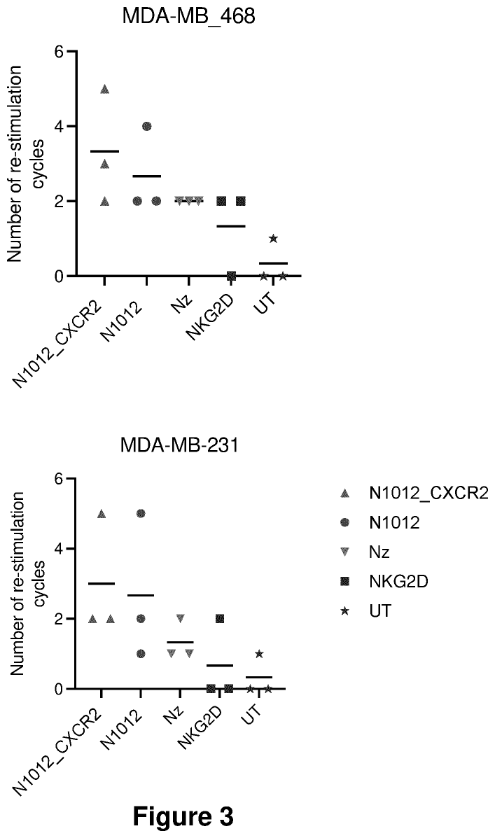

When compared to the N1012 and NKG2D T-cells, N1012_CXCR2 T-cells underwent significantly more rounds of re-stimulation upon MDA-MB-468 and MDA-MB-231 cells (FIG. 3).

### Example 3: Anti-Tumour Efficacy of N1012 CXCR2 T-Cells Activated Using Different Activation Stimuli

The anti-tumour in vivo efficacy of CAR T-cells activated using different activation stimuli was then evaluated. CAR T-cells were activated in vitro with PHA, the clinically compliant activation stimuli TransAct (Miltenyi Biotec GmbH) or the clinically compliant immobilised anti-CD3 and anti-CD28 antibodies. N1012 and non-transduced (UT) T-cells (also activated using the three different stimuli) were used for comparison.

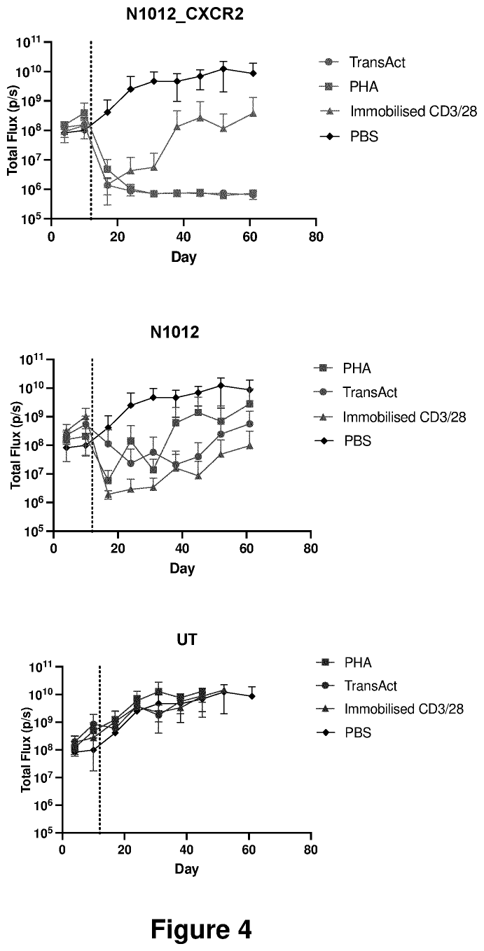

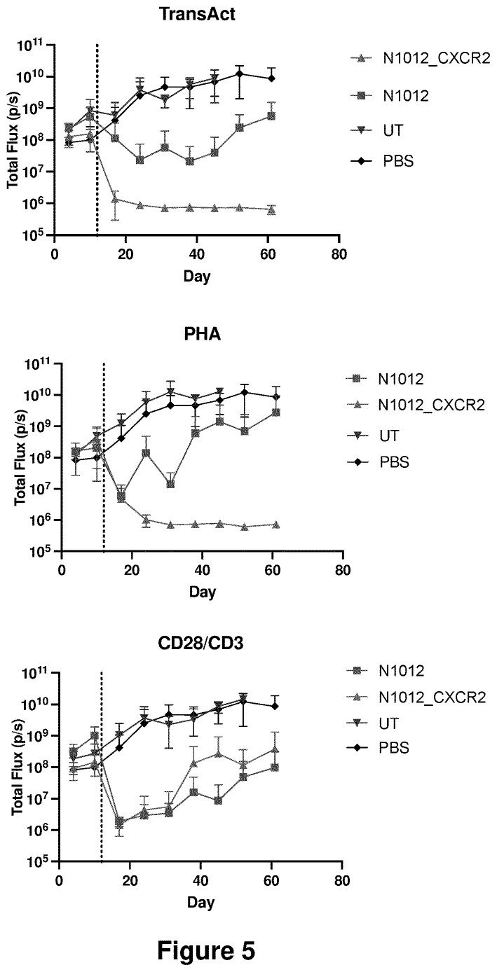

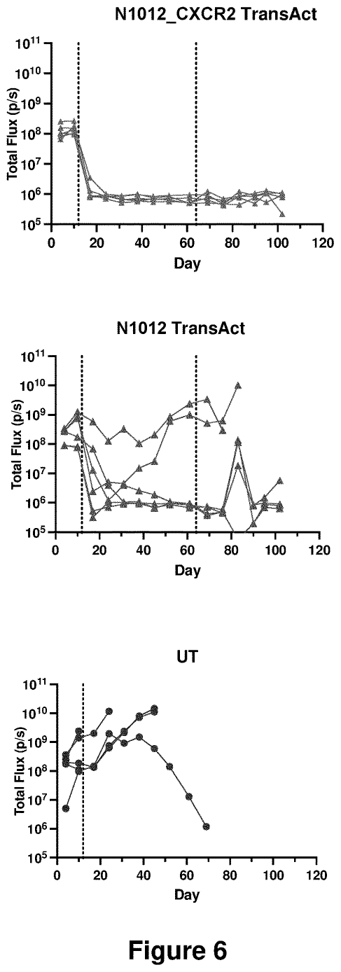

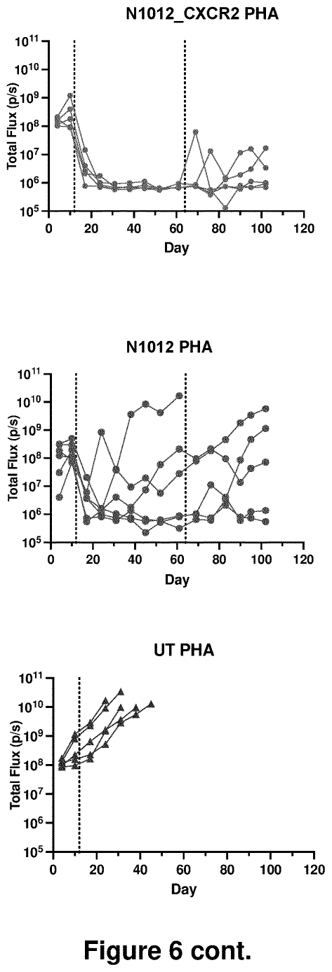

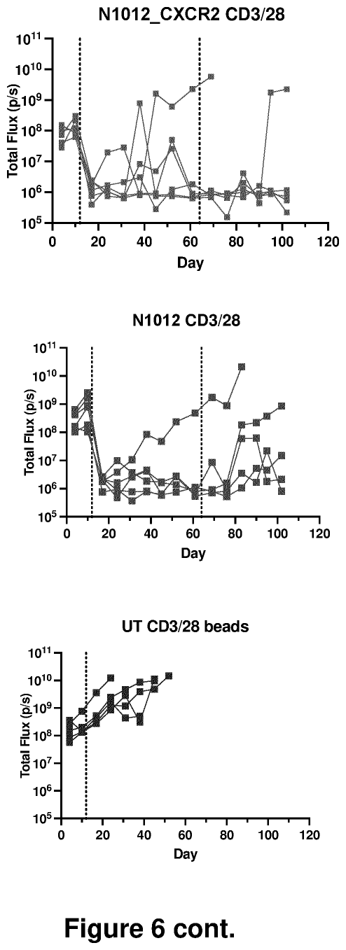

Briefly, 1×10⁵ firefly luciferase-tagged BxPC3 cells (BxPC3_LT) were administered into the peritoneal cavity of NSG mice. Twelve days post-tumour inoculation, 4×10⁶ T-cells (activated as described above) or PBS were administered to the mice. FIGS. 4 and 5 show that treatment efficacy was highest when CAR T-cells were activated with TransAct, especially N1012_CXCR2 CAR T-cells activated with TransAct. An additional rechallenge was undertaken on day 64 in mice that had achieved a complete response, as shown by the second broken vertical line in FIG. 6, which shows individual mice from FIGS. 4 and 5.

### Example 4: N1012 CXCR2 T-Cells Activated Using TransAct are Skewed Towards a CD4⁺ Profile

Structural features of the N1012_CXCR2 T-cells activated using different activation stimuli were then assessed. As in Example 3, CAR T-cells were activated in vitro with PHA or the clinically compliant activation stimuli TransAct. N1012, CYAD-01 replica cells and non-transduced (UT) T-cells (also activated using the two different stimuli) were used as comparators. The CYAD-01 CAR consists of a fusion of NKG2D to CD3Z and represents a human version of the mouse CAR originally described by Sentman et al. (Zhang et al., 2005, Blood 106:1544-1551). Although nominally a first-generation CAR, it associates with endogenous DAP10 in T-cells, meaning that both signals 1 and 2 are provided. The CYAD-01 CAR is currently undergoing clinical development by Celyad Oncology, and a replicate of this CAR is provided in these examples for the purposes of comparison only. In CYAD-01_10, a replica of Cyad-01 has been co-expressed with additional DAP10.

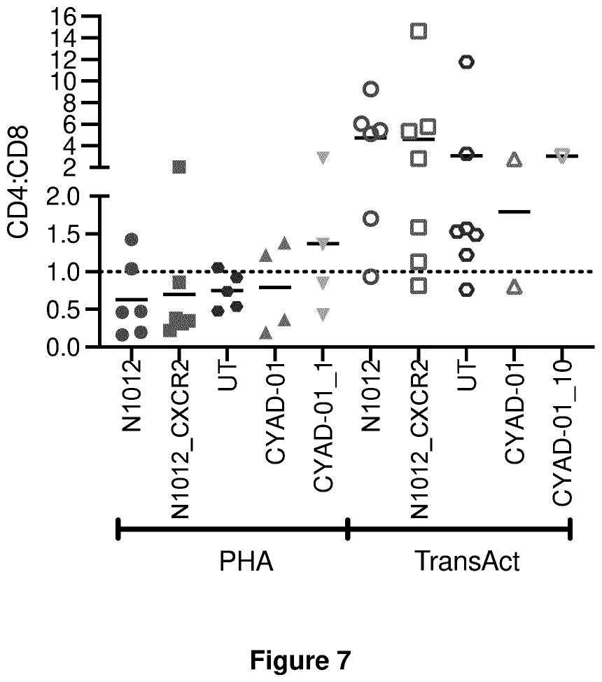

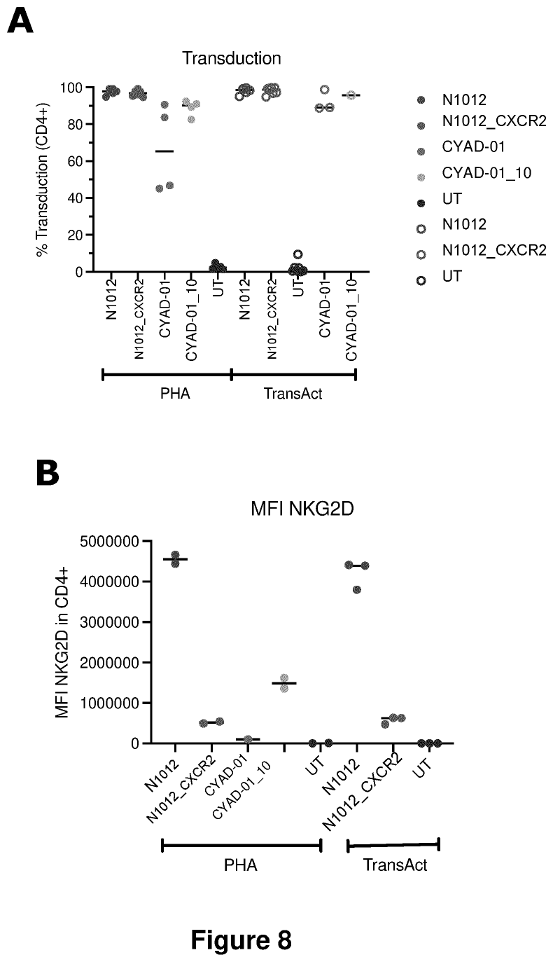

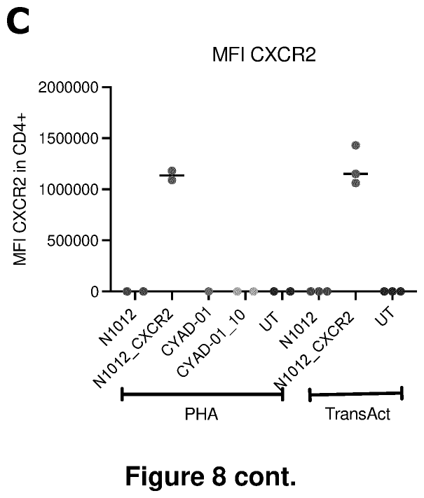

FIG. 7 shows that at the end of the in vitro culture period (day 10-12), N1012_CXCR2 T-cells activated with TransAct have a higher CD4 to CD8 ratio than N1012_CXCR2 T-cells activated with PHA. Whereas N1012_CXCR2 T-cells activated with PHA had more CD8 cells than CD4 cells, N1012_CXCR2 T-cells activated with TransAct had more CD4 than CD8 cells. This was unexpected. However, the transduction efficacy and median fluorescence intensity (MFI) of N1012_CXCR2 T-cells activated using TransAct was comparable to N1012_CXCR2 cells activated using PHA (FIG. 8).

The fold expansion of T-cells activated with either 5 ug/mL phytohaemagglutinin-L (PHA) or 10 uL TransAct reagent per 1×10⁶ PBMCs was then assessed. This was calculated by dividing the number of T-cells present at the end of the ex vivo culture period (of 10 to 12 days) by the number of T-cells initially transduced.

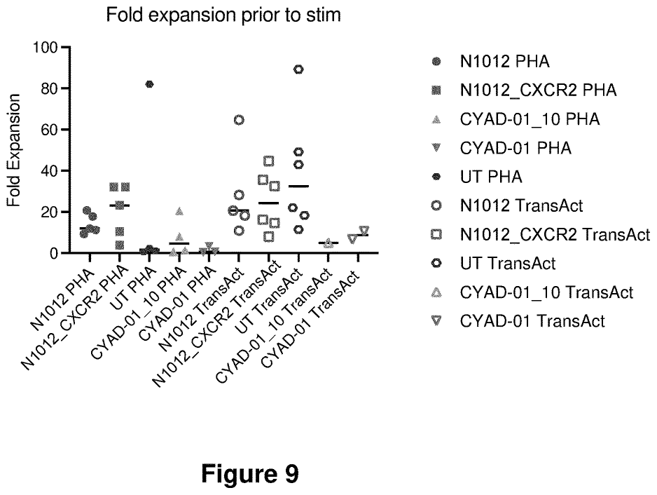

In addition to a different CD4:CD8 ratio, cells activated with TransAct had a greater fold expansion than cells activated with PHA (FIG. 9).

### Example 5: Expression of CARs in γδ T-Cells

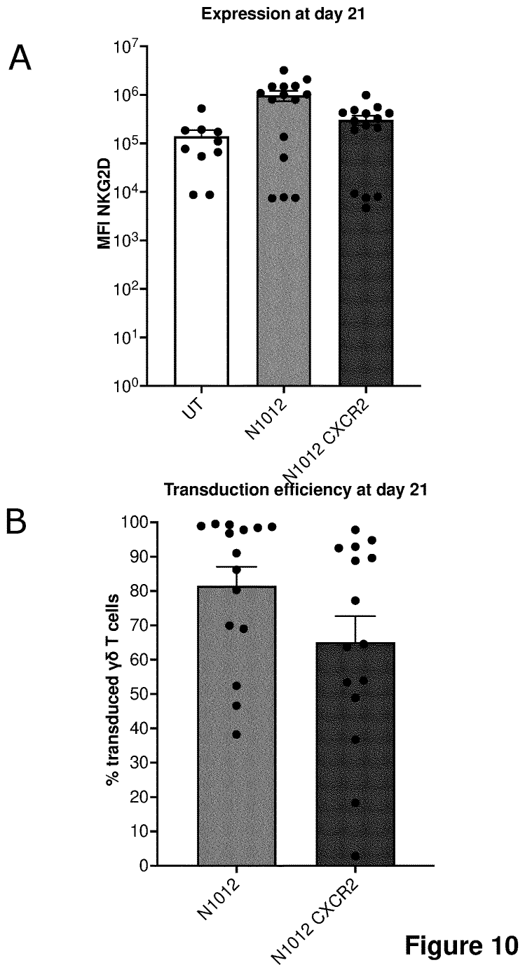

Primary human γδ T-cells were activated using an immobilised pan-gamma-delta TCR antibody (clone 11F2). Forty-eight hours after activation, T-cells were engineered by retroviral transduction to express N1012 or N1012_CXCR2. Surface expression of NKG2D was assessed by flow cytometry, co-staining for TCR and CD3 expression. The median fluorescence intensity (MFI, FIG. 10A) and percentage expression (FIG. 10B) of NKG2D was compared against untransduced T-cells. As shown, both N1012 and N1012_CXCR2 constructs are reproducibly expressed at high levels at the surface of primary human γδ T-cells.

### Example 6: Assessment of N1012 and N1012 CXCR2 γδ T-Cell Expansion and Anti-Tumour Cytotoxicity

The in vitro functionality of N1012 or N1012_CXCR2 expressing γδ T-cells was then assessed.

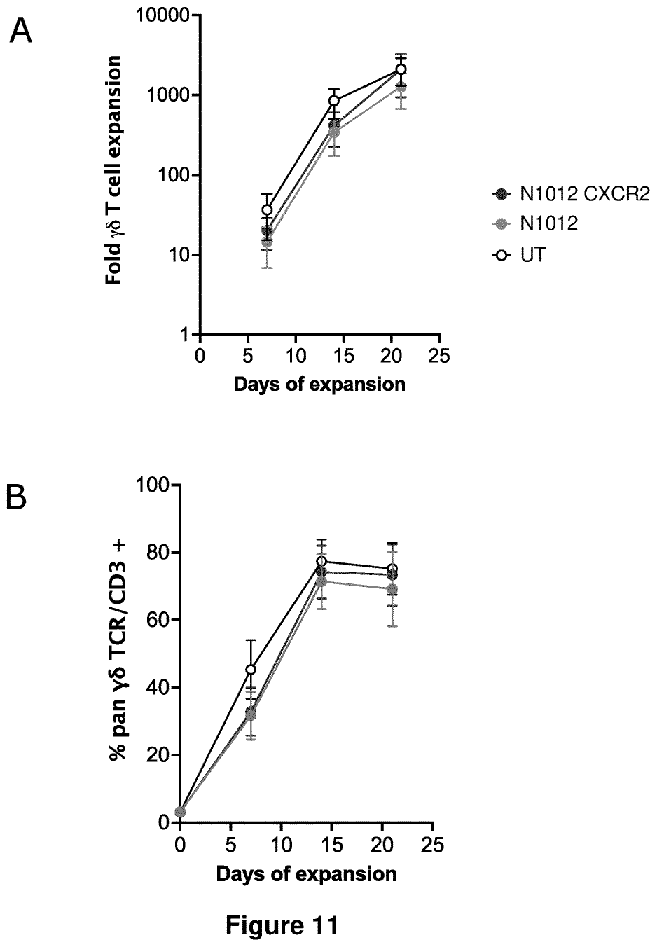

To assess fold expansion, N1012 expressing γδ T-cells or N1012_CXCR2 expressing γδ T-cells were activated as described above. Fold expansions, as shown in FIG. 11A, were calculated on the basis of % pan-γδ TCR⁺ CD3⁺ cells at day 0, day 7, day 14 and day 21 in culture. FIG. 11B shows the % of pan-γδ TCR⁺ CD3⁺ cells at day 0, day 7, day 14 and day 21 (n=11). As FIG. 11 demonstrates, both the % of cells and the fold expansion rate were similar in untransduced, N1012 and N1012_CXCR2 expressing γδ T-cells.

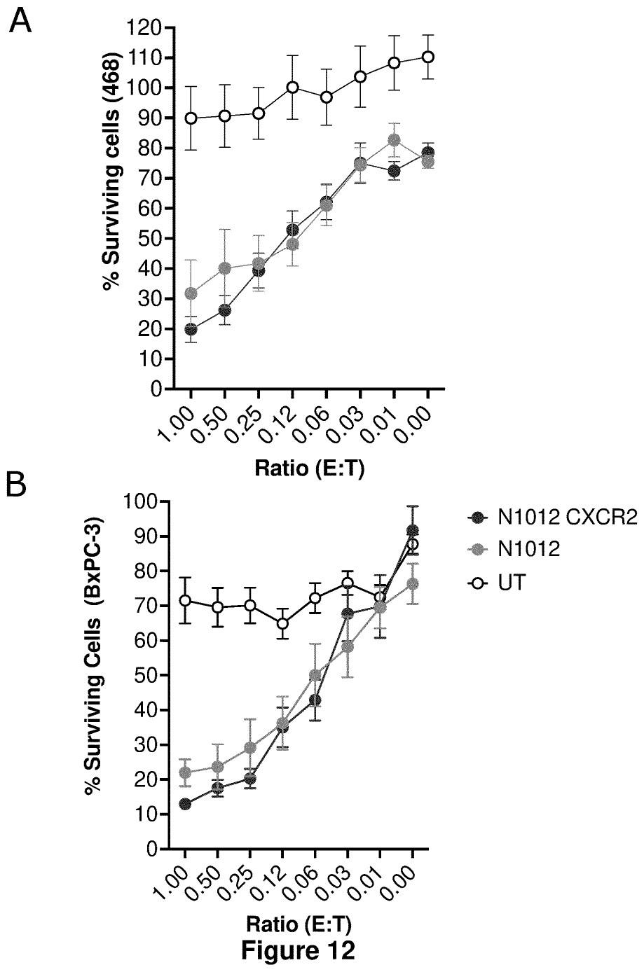

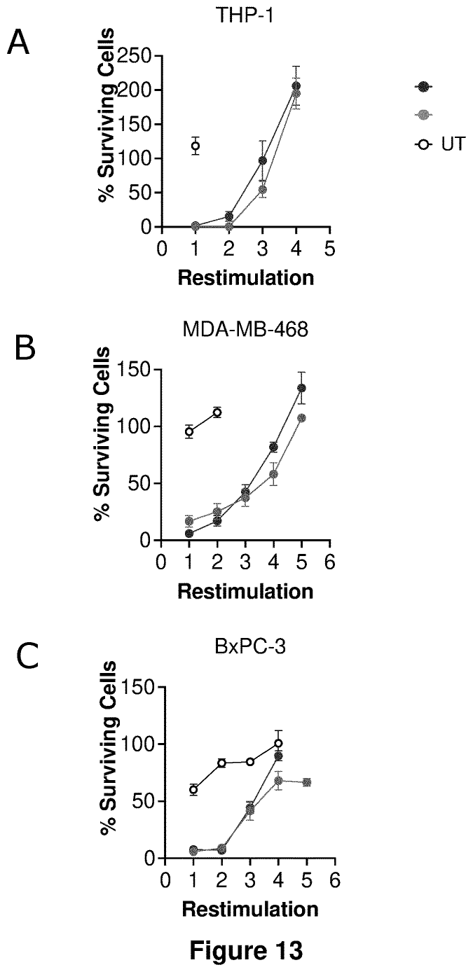

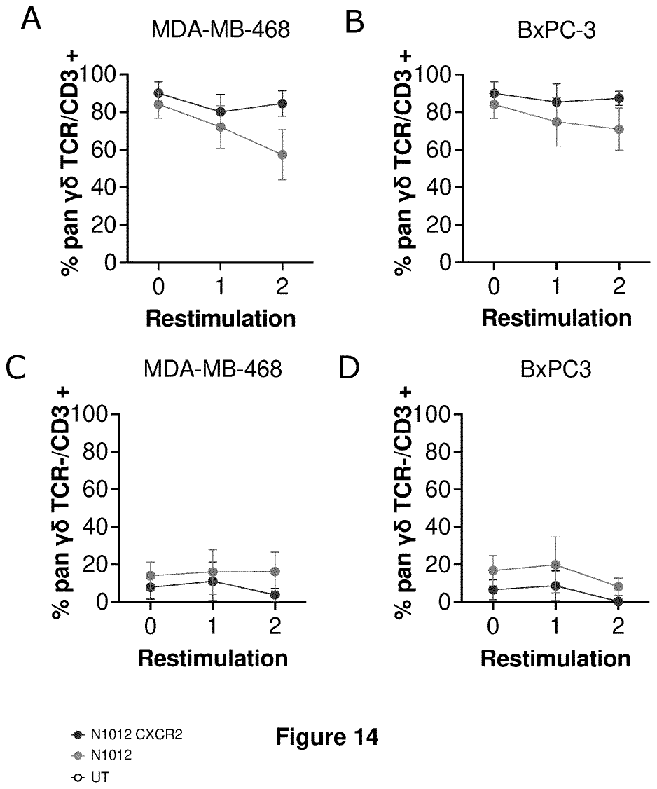

The in vitro anti-tumour functionality of the N1012 and N1012_CXCR2 expressing γδ T-cells was then considered. Dose response assays measuring the cytotoxicity of untransduced (UT) and CAR⁺ (N1012 or N1012_CXCR2) γδ T-cells against the triple negative breast cancer cell line MDA-MB-468 at a range of effector to target ratios are shown in FIG. 12A. Dose response assays measuring the cytotoxicity of untransduced (UT) and CAR⁺ (N1012 or N1012_CXCR2) γδ T-cells against the pancreatic cancer cell line BxPC-3 at a range of effector to target ratios (n=3) are shown in FIG. 12B. Both N1012 and N1012_CXCR2 γδ T-cells exhibited clear cytotoxicity against the cancer cell lines. This cytotoxicity was maintained after numerous T-cell restimulations, including against the acute myeloid leukaemia AML cell line THP-1-LT (n=2) (FIG. 13). The purity of the CAR⁺ γδ T-cells following restimulation on MDA-MB-468 cells or BxPC-3 cells is shown in FIGS. 14A and 14B, respectively. The percentage of αβ T-cells following restimulation on MDA-MB-468 or BxPC-3 cells remained low, as shown in FIGS. 14C and 14D, respectively.

### Example 7: Anti-Tumour In Vivo Efficacy of N1012 and N1012 CXCR2 γδ T-Cells

The in vivo anti-tumour efficacy of N1012 and N1012_CXCR2 γδ T-cells was then assessed.

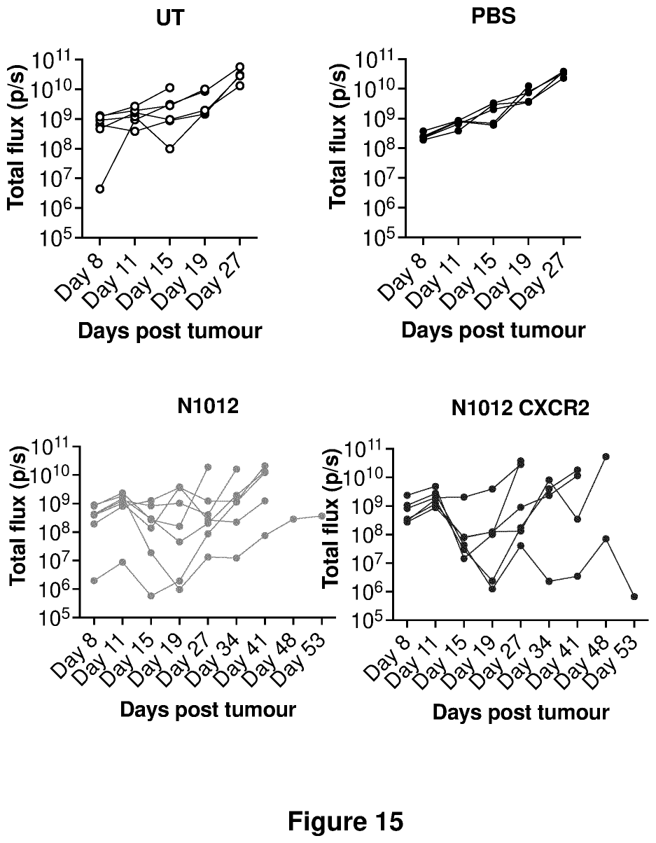

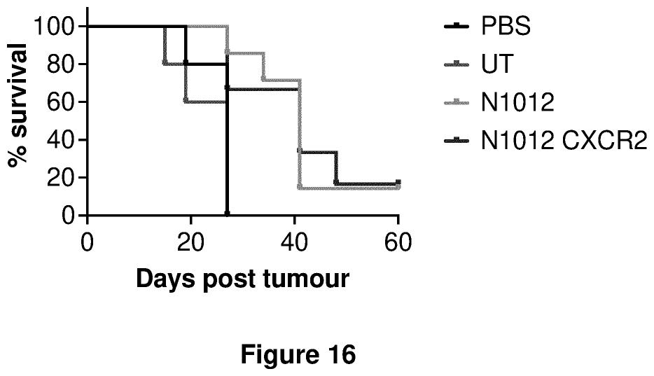

1×10⁵ firefly luciferase-tagged BxPC3 cells (BxPC3_LT) were administered into the peritoneal cavity of NSG mice. Eleven days post-tumour inoculation, 1×10⁷ of CAR⁺ γδ T-cells were administered to the mice. Non-transduced γδ T-cells and PBS were used as controls. Results are shown in FIG. 15. N1012 or N1012_CXCR2 (N1012 and N1012_CXCR2, respectively) γδ T-cells reduced tumour burden in mice (FIG. 15) and prolonged mouse survival compared to mice administered control PBS or untransduced γδ T-cells (FIG. 16).

### Example 8: N1012 CXCR2 T-Cells Exhibit Consistently Enhanced Anti-Tumour Efficacy

Survival curves from various in vivo experiments were pooled. In particular, survival curves from CFPac1 (pancreatic, two models), BxPC3 (pancreatic, two models), Kuramochi (High grade serous ovarian, one model), Ovsaho (epithelial ovarian cancer, one model), Mesothelioma (patient-derived xenograft, one model), triple-negative breast cancer (patient-derived xenograft, one model) or SKOV3 (epithelial ovarian cancer, one model) tumour xenograft-containing mice treated with PBS or 1×10⁷ CAR T-cells (N1012, N1012_CXCR2, or untransduced) were pooled.

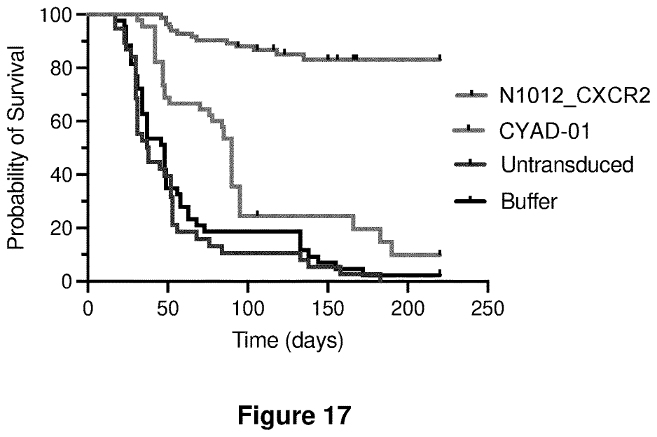

These are shown in FIG. 17. As noted above, CAR T-cells (N1012_CXCR2, CYAD-01 or PBS buffer as control) were injected into mice bearing CFPac-1 tumour cells or BxPC3 cells tumour cells.

As FIG. 17 shows, the probability of survival was markedly enhanced for mice administered N1012_CXCR2 cells, compared to other cells. This is further demonstrated by Table 4, which shows the median survival from the pooled data for each cell group.

TABLE 4
Pooled median survival

| | Median survival | Survival |
|---|---|---|
| Construct | (days) | probability |
| N1012_CXCR2 | Not yet reached | 83% |
| CYAD-01 analogue | 90 | 10% |
| Untransduced (UT) | 38 | 0% |
| PBS | 48 | 2% |

### Example 9: Efficient Transduction and Expansion of N1012 CXCR2 can be Achieved Across a Range of TransAct™ Concentrations, Including Those Substantially Below the Concentration Recommended by the Manufacturer

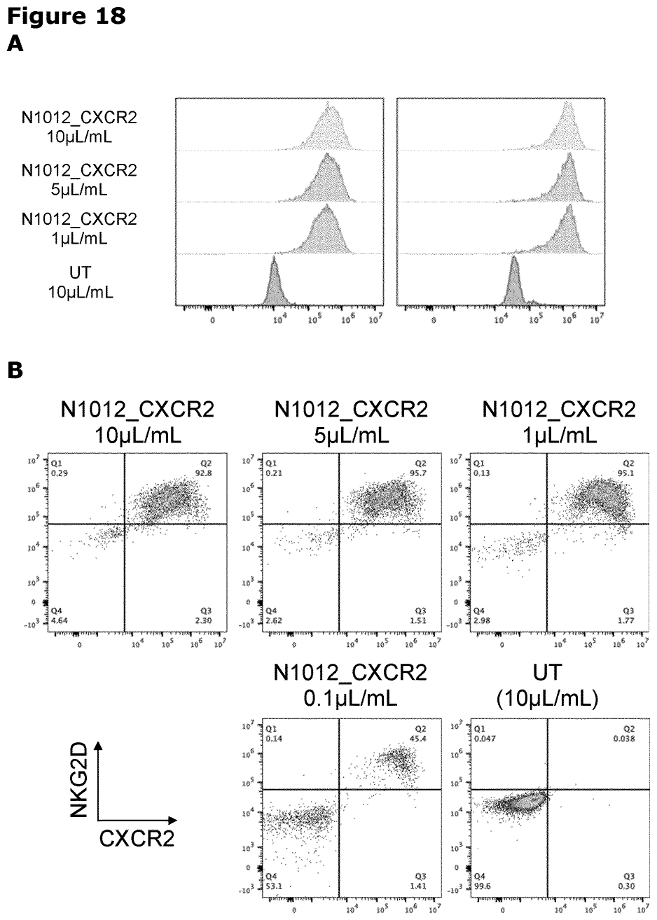

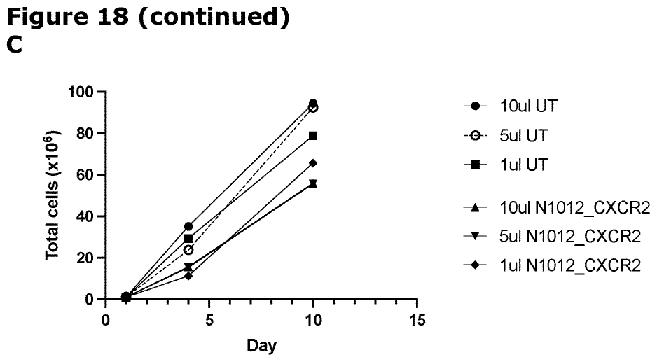

Primary human peripheral blood mononuclear cells (at a concentration of 1×10⁶/mL) were activated with TransAct™ at a range of concentrations between 0.1 μL/mL and the manufacturer's recommended concentration of 10 μL/mL. Seventy-two hours after activation, the cells were enumerated by trypan blue exclusion and 5×10⁵ either left untransduced, or transduced with N1012_CXCR2 retrovirus. Transduction efficiency, as measured by CXCR2 and/or NKG2D expression in CD4+ T-cells was assessed three and ten days post-transduction by flow cytometry, using fluorescein isothiocyanate (FITC)-conjugated anti-human CD4, allophycocyanin-cyanine7 (APC-Cy7)-conjugated anti-human CD8, phycoerythrin (PE)-conjugated anti-human NKG2D and AlexaFluor647-conjugated anti-human CXCR2 antibodies, as detailed in FIG. 18.

Expansion of the untransduced (UT) and N1012_CXCR2 T-cells was assessed three and ten days post-transduction by trypan blue exclusion. The cell concentration was multiplied by total culture volume to provide a definitive cell number.

The results show that good transduction is obtained, even when using only 10% of the manufacturer recommended concentration of TransAct™.

### Example 10: Anti-Tumour Efficacy of N1012 CXCR2 T-Cells in Metastatic Colorectal Carcinoma Xenografts Models

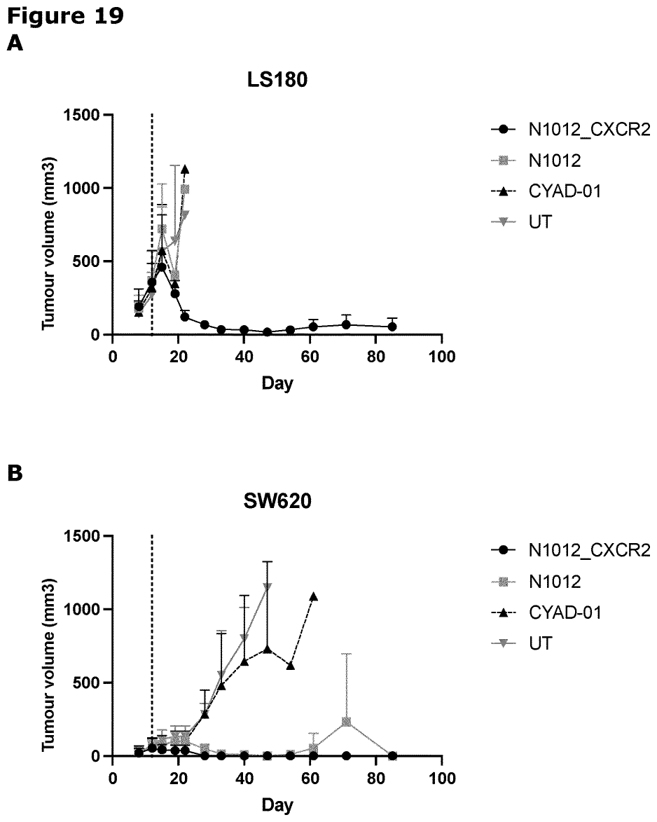

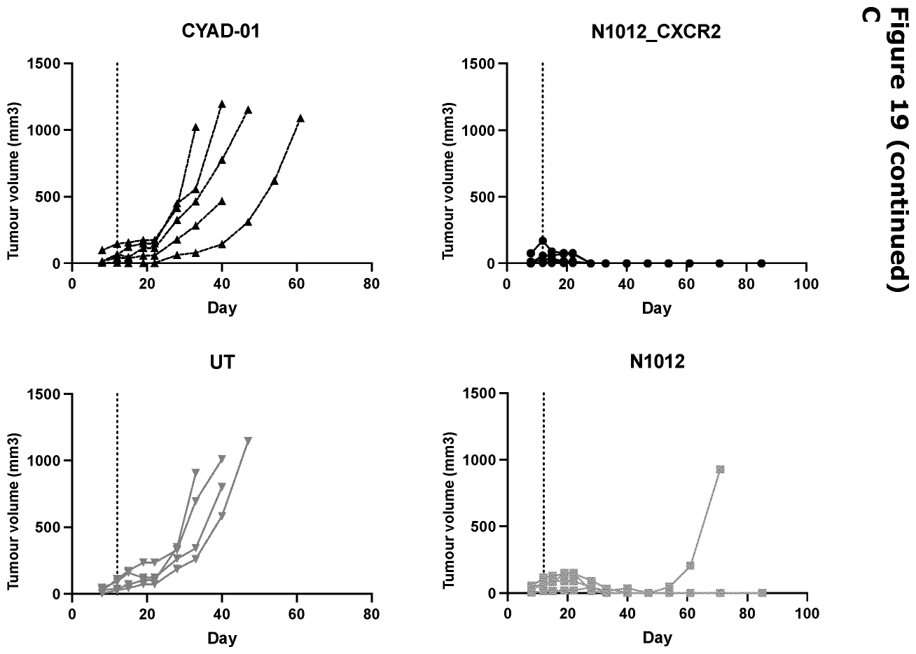

1×10⁶ LS180 or SW620 tumour cells were injected subcutaneously into the left flank of NSG mice. Tumour growth was measured using weekly caliper measurements. Twelve days post-engraftment, mice were treated with 1×10⁷ CAR⁺ T-cells or 1×10⁷ untransduced T-cells. LS180 tumour bearing mice received an intravenous dose of either N1012_CXCR2 (n=6), N1012 (n=7), CYAD-01 (n=6), or untransduced (UT) T-cells (n=5). SW620 tumour bearing mice received either N1012_CXCR2 (n=5), N1012 (n=4), CYAD-01 (n=5), or UT T-cells (n=4). Results shown in FIG. 19 demonstrate that N1012_CXCR2 achieves robust anti-tumour efficacy against both models.

SEQUENCES

SEQ ID NO: 1 (human DAP10 full sequence)
MIHLGHILFL LLLPVAAAQT TPGERSSLPA FYPGTSGSCS GCGSLSLPLL AGLVAADAVA
SLLIVGAVEL CARPRRSPAQ EDGKVYINMP GRG
SEQ ID NO: 2 (DAP10 aa19-93-lacking leader sequence)
QTTPGERSSL PAFYPGTSGS CSGCGSLSLP LLAGLVAADA VASLLIVGAV FLCARPRRSP
AQEDGKVYIN MPGRG
SEQ ID NO: 3 (DAP10 aa19-69-extracellular/transmembrane domain)
QTTPGERSSL PAFYPGTSGS CSGCGSLSLP LLAGLVAADA VASLLIVGAV F
SEQ ID NO: 4 (DAP10 aa1-71)
MIHLGHILFL LLLPVAAAQT TPGERSSLPA FYPGTSGSCS GCGSLSLPLL AGLVAADAVA
SLLIVGAVEL C
SEQ ID NO: 5 (DAP10 aa19-71)
QTTPGERSSL PAFYPGTSGS CSGCGSLSLP LLAGLVAADA VASLLIVGAV FLC
SEQ ID NO: 6 (DAP10 aa70-93-intracellular domain)
LCARPRRSPA QEDGKVYINM PGRG
SEQ ID NO: 7 (DAP10 aa49-93-transmembrane and intracellular domain)
LLAGLVAADA VASLLIVGAV FLCARPRRSP AQEDGKVYIN MPGRG
SEQ ID NO: 8 (DAP10 aa49-69-transmembrane domain)
LLAGLVAADA VASLLIVGAV F
SEQ ID NO: 9 (human DAP12 full sequence)
MGGLEPCSRL LLLPLLLAVS GLRPVQAQAQ SDCSCSTVSP GVLAGIVMGD LVLTVLIALA
VYFLGRLVPR GRGAAEAATR KQRITETESP YQELQGQRSD VYSDLNTQRP YYK
SEQ ID NO: 10 (DAP12 aa22-113-lacking leader sequence)
LRPVQAQAQS DCSCSTVSPG VLAGIVMGDL VLTVLIALAV YFLGRLVPRG RGAAEAATRK
QRITETESPY QELQGQRSDV YSDLNTQRPY YK
SEQ ID NO: 11 (DAP12 aa62-113-cytoplasmic/intracellular domain)
YFLGRLVPRG RGAAEAATRK QRITETESPY QELQGQRSDV YSDLNTQRPY YK
SEQ ID NO: 12 (DAP12 aa41-61-transmembrane domain)
GVLAGIVMGD LVLTVLIALA V
SEQ ID NO: 13 (DAP12 aa22-61-extracellular and transmembrane domains)
LRPVQAQAQS DCSCSTVSPG VLAGIVMGDL VLTVLIALAV
SEQ ID NO: 14 (human NKG2D full sequence)
MGWIRGRRSR HSWEMSEFHN YNLDLKKSDF STRWQKQRCP VVKSKCRENA SPFFFCCFIA
VAMGIRFIIM VAIWSAVFLN SLENQEVQIP LTESYCGPCP KNWICYKNNC YQFFDESKNW
YESQASCMSQ NASLLKVYSK EDQDLLKLVK SYHWMGLVHI PTNGSWQWED GSILSPNLLT
IIEMQKGDCA LYASSFKGYI ENCSTPNTYI CMQRTV
SEQ ID NO: 15 (human NKG2D aa73-216-extracellular domain)
IWSAVFLNSL FNQEVQIPLT ESYCGPCPKN WICYKNNCYQ FFDESKNWYE SQASCMSQNA
SLLKVYSKED QDLLKLVKSY HWMGLVHIPT NGSWQWEDGS ILSPNLLTII EMQKGDCALY
ASSFKGYIEN CSTPNTYICM QRTV
SEQ ID NO: 16 (human NKG2D aa82-216-extracellular domain)
LENQEVQIPL TESYCGPCPK NWICYKNNCY QFFDESKNWY ESQASCMSQN ASLLKVYSKE
DQDLLKLVKS YHWMGLVHIP TNGSWQWEDG SILSPNLLTI IEMQKGDCAL YASSFKGYIE
NCSTPNTYIC MQRTV
SEQ ID NO: 17 (human NKG2D aa52-216-transmembrane and extracellular domain)
PFFFCCFIAV AMGIRFIIMV AIWSAVELNS LFNQEVQIPL TESYCGPCPK NWICYKNNCY QFFDESKNWY
ESQASCMSQN ASLLKVYSKE DQDLLKLVKS YHWMGLVHIP TNGSWQWEDG SILSPNLLTI IEMQKGDCAL
YASSFKGYIE NCSTPNTYIC MQRTV
SEQ ID NO: 18 (linker)
GSG
SEQ ID NO: 19 (linker)
GSGGG
SEQ ID NO: 20 (linker)
GSGG
SEQ ID NO: 21 (linker)
SGGG
SEQ ID NO: 22 (linker)
GGGGS
SEQ ID NO: 23 (linker)
GGGGSGGGGSGGGGSGGGGS
SEQ ID NO: 24 (linker)
GGGGSGGGGSGGGGS
SEQ ID NO: 25 (linker)
GPPGS
SEQ ID NO: 26 (linker)
GGGS
SEQ ID NO: 27 (linker)
GGGGS
SEQ ID NO: 28 (linker)
GYS
SEQ ID NO: 29 (linker)
GS
SEQ ID NO: 30 (linker)
SGGGG
SEQ ID NO: 31 (linker)
SGGG
SEQ ID NO: 32 (linker)
SGG
SEQ ID NO: 33 (linker)
SGSG
SEQ ID NO: 34 (linker)
SG
SEQ ID NO: 35 (linker)
GGGGA
SEQ ID NO: 36 (linker)
GGGA
SEQ ID NO: 37 (linker)
EAAAK
SEQ ID NO: 38 (furin cleavage site)
RRKR
SEQ ID NO: 39 (P2A skip peptide)
ATNFSLLKQAGDVEENPGP
SEQ ID NO: 40 (T2A skip peptide)
EGRGSLLTCGDVEENPGP
SEQ ID NO: 41 (SGSG + P2A)
SGSGATNFSLLKQAGDVEENPGP
SEQ ID NO: 42 (SGSG + T2A)
SGSGEGRGSLLTCGDVEENPGP
SEQ ID NO: 43 (furin + SGSG + P2A)
RRKRSGSGATNFSLLKQAGDVEENPGP
SEQ ID NO: 44 (furin + SGSG + T2A)
RRKRSGSGEGRGSLLTCGDVEENPGP
SEQ ID NO: 45 (F2A skip peptide)
VKQTLNFDLLKLAGDVESNPGP
SEQ ID NO: 46 (E2A skip peptide)
QCTNYALLKLAGDVESNPGP
SEQ ID NO: 47 (His tag)
HHHHHH
SEQ ID NO: 48 (FLAG tag)
DYKDDDDK
SEQ ID NO: 49 (Avi tag)
GLNDIFEAQKIEWHE
SEQ ID NO: 50 (V5 tag)
GKPIPNPLLGLDST
SEQ ID NO: 51 (V5 tag)
IPNPLLGLD
SEQ ID NO: 52 (Myc tag)
EQKLISEEDL
SEQ ID NO: 53 (AHF tag)
GLNDIFEAQKIEWHEGGHHHHHHDYKDDDDK
SEQ ID NO: 54 (FHA tag)
DYKDDDDKHHHHHHGGGLNDIFEAQKIEWHE
SEQ ID NO: 55 (CD8α leader sequence)
MALPVTALLL PLALLLHAAR P
SEQ ID NO: 56 (4-1BB endodomain)
KRGRKKLLYI FKQPFMRPVQ TTQEEDGCSC RFPEEEEGGC EL
SEQ ID NO: 57 (CD27 endodomain)
QRRKYRSNKG ESPVEPAEPC HYSCPREEEG STIPIQEDYR KPEPACSP
SEQ ID NO: 58 (human IgG1 hinge-aa 218-229 of UniProt: P0DOX5)
EPKSCDKTHT CP
SEQ ID NO: 59 (truncated CD8α hinge)
TTTPAPRPPT PAPTIASQPL SLRPEACRPA AGGAVHTRGL DFACD
SEQ ID NO: 60
MIHLGHILFLLLLPVAAAQTTPGERSSLPAFYPGTSGSCSGCGSLSLPLLAGLVAADAVASLLIVGAVELCARPRRS
PAQEDGKVYINMPGRGYFLGRLVPRGRGAAEAATRKQRITETESPYQELQGQRSDVYSDLNTQRPYYK
SEQ ID NO: 61
MALPVTALLLPLALLLHAARPDYKDDDDKQTTPGERSSLPAFYPGTSGSCSGCGSLSLPLLAGLVAADAVASLLIVG
AVFYFLGRLVPRGRGAAEAATRKQRITETESPYQELQGQRSDVYSDLNTQRPYYK
SEQ ID NO: 62
MALPVTALLLPLALLLHAARPDYKDDDDKEPKSCDKTHTCPLLAGLVAADAVASLLIVGAVFLCARPRRSPAQEDGK
VYINMPGRGYFLGRLVPRGRGAAEAATRKQRITETESPYQELQGQRSDVYSDLNTQRPYYK
SEQ ID NO: 63
MALPVTALLLPLALLLHAARPDYKDDDDKTTTPAPRPPTPAPTIASQPLSLRPEACRPAAGGAVHTRGLDFACDLLA
GLVAADAVASLLIVGAVFLCARPRRSPAQEDGKVYINMPGRGYFLGRLVPRGRGAAEAATRKQRITETESPYQELQG
QRSDVYSDLNTQRPYYK
SEQ ID NO: 64 (Construct 1/N1012)
MIHLGHILFLLLLPVAAAQTTPGERSSLPAFYPGTSGSCSGCGSLSLPLLAGLVAADAVASLLIVGAVFLCARPRRS
PAQEDGKVYINMPGRGYFLGRLVPRGRGAAEAATRKQRITETESPYQELQGQRSDVYSDLNTQRPYYKRRKRSGSGA
TNFSLLKQAGDVEENPGPMGWIRGRRSRHSWEMSEFHNYNLDLKKSDFSTRWQKQRCPVVKSKCRENASPFFFCCFI
AVAMGIRFIIMVAIWSAVELNSLFNQEVQIPLTESYCGPCPKNWICYKNNCYQFFDESKNWYESQASCMSQNASLLK
VYSKEDQDLLKLVKSYHWMGLVHIPTNGSWQWEDGSILSPNLLTIIEMQKGDCALYASSFKGYIENCSTPNTYICMQ
RTV
SEQ ID NO: 65 (Construct 3)
MALPVTALLLPLALLLHAARPDYKDDDDKQTTPGERSSLPAFYPGTSGSCSGCGSLSLPLLAGLVAADAVASLLIVG
AVFYFLGRLVPRGRGAAEAATRKQRITETESPYQELQGQRSDVYSDLNTQRPYYKRRKRSGSGEGRGSLLTCGDVEE
NPGPMIHLGHILFLLLLPVAAAQTTPGERSSLPAFYPGTSGSCSGCGSLSLPLLAGLVAADAVASLLIVGAVFLCAR
PRRSPAQEDGKVYINMPGRGRRKRSGSGATNFSLLKQAGDVEENPGPMGWIRGRRSRHSWEMSEFHNYNLDLKKSDF
STRWQKQRCPVVKSKCRENASPFFFCCFIAVAMGIRFIIMVAIWSAVELNSLFNQEVQIPLTESYCGPCPKNWICYK
NNCYQFFDESKNWYESQASCMSQNASLLKVYSKEDQDLLKLVKSYHWMGLVHIPTNGSWQWEDGSILSPNLLTIIEM
QKGDCALYASSFKGYIENCSTPNTYICMQRTV
SEQ ID NO: 66 (Construct 8)
MIHLGHILFLLLLPVAAAQTTPGERSSLPAFYPGTSGSCSGCGSLSLPLLAGLVAADAVASLLIVGAVFLCARPRRS
PAQEDGKVYINMPGRGYFLGRLVPRGRGAAEAATRKQRITETESPYQELQGQRSDVYSDLNTQRPYYKRRKRSGSGA
TNFSLLKQAGDVEENPGPMGWIRGRRSRHSWEMSEFHNYNLDLKKSDESTRWQKQRCPVVKSKCRENASPFFFCCFI
AVAMGIRFIIMVAIWSAVELNSLFNQEVQIPLTESYCGPCPKNWICYKNNCYQFFDESKNWYESQASCMSQNASLLK
VYSKEDQDLLKLVKSYHWMGLVHIPTNGSWQWEDGSILSPNLLTIIEMQKGDCALYASSFKGYIENCSTPNTYICMQ
RTVRRKRSGSGEGRGSLLTCGDVEENPGPMIHLGHILFLLLLPVAAAQTTPGERSSLPAFYPGTSGSCSGCGSLSLP
LLAGLVAADAVASLLIVGAVFLCKRGRKKLLYIFKQPFMRPVQTTQEEDGCSCRFPEEEEGGCEL
SEQ ID NO: 67 (Construct 9)
MIHLGHILFLLLLPVAAAQTTPGERSSLPAFYPGTSGSCSGCGSLSLPLLAGLVAADAVASLLIVGAVELCARPRRS
PAQEDGKVYINMPGRGYFLGRLVPRGRGAAEAATRKQRITETESPYQELQGQRSDVYSDLNTQRPYYKRRKRSGSGA
TNFSLLKQAGDVEENPGPMGWIRGRRSRHSWEMSEFHNYNLDLKKSDFSTRWQKQRCPVVKSKCRENASPFFFCCFI
AVAMGIRFIIMVAIWSAVELNSLFNQEVQIPLTESYCGPCPKNWICYKNNCYQFFDESKNWYESQASCMSQNASLLK
VYSKEDQDLLKLVKSYHWMGLVHIPTNGSWQWEDGSILSPNLLTIIEMQKGDCALYASSFKGYIENCSTPNTYICMQ
RTVRRKRSGSGEGRGSLLTCGDVEENPGPMALPVTALLLPLALLLHAARPDYKDDDDKQTTPGERSSLPAFYPGTSG
SCSGCGSLSLPLLAGLVAADAVASLLIVGAVFLCKRGRKKLLYIFKQPFMRPVQTTQEEDGCSCRFPEEEEGGCEL
SEQ ID NO: 68 (Construct 10)
MIHLGHILFLLLLPVAAAQTTPGERSSLPAFYPGTSGSCSGCGSLSLPLLAGLVAADAVASLLIVGAVELCARPRRS
PAQEDGKVYINMPGRGYFLGRLVPRGRGAAEAATRKQRITETESPYQELQGQRSDVYSDLNTQRPYYKRRKRSGSGA
TNFSLLKQAGDVEENPGPMGWIRGRRSRHSWEMSEFHNYNLDLKKSDFSTRWQKQRCPVVKSKCRENASPFFFCCFI
AVAMGIRFIIMVAIWSAVELNSLFNQEVQIPLTESYCGPCPKNWICYKNNCYQFFDESKNWYESQASCMSQNASLLK
VYSKEDQDLLKLVKSYHWMGLVHIPTNGSWQWEDGSILSPNLLTIIEMQKGDCALYASSFKGYIENCSTPNTYICMQ
RTVRRKRSGSGEGRGSLLTCGDVEENPGPMIHLGHILFLLLLPVAAAQTTPGERSSLPAFYPGTSGSCSGCGSLSLP
LLAGLVAADAVASLLIVGAVFLCQRRKYRSNKGESPVEPAEPCHYSCPREEEGSTIPIQEDYRKPEPACSP
SEQ ID NO: 69 (Construct 11)
MIHLGHILFLLLLPVAAAQTTPGERSSLPAFYPGTSGSCSGCGSLSLPLLAGLVAADAVASLLIVGAVFLCARPRRS
PAQEDGKVYINMPGRGYFLGRLVPRGRGAAEAATRKQRITETESPYQELQGQRSDVYSDLNTQRPYYKRRKRSGSGA
TNFSLLKQAGDVEENPGPMGWIRGRRSRHSWEMSEFHNYNLDLKKSDFSTRWQKQRCPVVKSKCRENASPFFFCCFI
AVAMGIRFIIMVAIWSAVFLNSLFNQEVQIPLTESYCGPCPKNWICYKNNCYQFFDESKNWYESQASCMSQNASLLK
VYSKEDQDLLKLVKSYHWMGLVHIPTNGSWQWEDGSILSPNLLTIIEMQKGDCALYASSFKGYIENCSTPNTYICMQ
RTVRRKRSGSGEGRGSLLTCGDVEENPGPMALPVTALLLPLALLLHAARPDYKDDDDKQTTPGERSSLPAFYPGTSG
SCSGCGSLSLPLLAGLVAADAVASLLIVGAVFLCQRRKYRSNKGESPVEPAEPCHYSCPREEEGSTIPIQEDYRKPE
PACSP
SEQ ID NO: 70 (encoding polypeptide of SEQ ID NO: 60)
ATGATCCACCTGGGCCACATCCTGTTCCTGCTGCTGCTGCCCGTGGCCGCTGCCCAGACCACCCCTGGCGAGCGGAG
CAGCCTGCCTGCCTTCTACCCTGGCACCAGCGGCAGCTGCAGCGGCTGCGGCAGCCTGAGCCTGCCCCTGCTGGCCG
GCCTGGTGGCCGCCGACGCCGTGGCCAGCCTGCTGATCGTGGGCGCCGTGTTCCTGTGCGCCAGGCCCAGGCGGAGC
CCtGCCCAGGAGGACGGCAAGGTGTACATCAACATGCCCGGCCGGGGCTACTTCCTGGGCAGGCTGGTGCCCAGGGG
CAGGGGCGCTGCCGAGGCTGCCACCCGGAAGCAGCGGATCACCGAGACCGAGAGCCCCTACCAGGAGCTGCAGGGCC
AGCGGAGCGACGTGTACAGCGACCTGAACACCCAGAGGCCCTACTACAAG
SEQ ID NO: 71 (encoding polypeptide of SEQ ID NO: 61)
ATGGCTCTGCCTGTGACAGCTCTGCTGCTGCCTCTGGCTCTGCTGCTGCACGCCGCTAGACCCGATTATAAGGACGA
CGACGACAAGCAGACCACCCCTGGCGAGCGGAGCAGCCTGCCTGCCTTCTACCCTGGCACCAGCGGCAGCTGCAGCG
GCTGCGGCAGCCTGAGCCTGCCCCTGCTGGCtGGCCTGGTGGCCGCCGACGCCGTGGCCAGCCTGCTGATCGTGGGC
GCCGTGTTCTACTTCCTGGGCAGGCTGGTGCCCAGGGGCAGGGGCGCTGCCGAGGCTGCCACCCGGAAGCAGCGGAT
CACCGAGACCGAGAGCCCCTACCAGGAGCTGCAGGGCCAGCGGAGCGACGTGTACAGCGACCTGAACACCCAGAGGC
CCTACTACAAG
SEQ ID NO: 72 (encoding polypeptide of SEQ ID NO: 62)
ATGGCTCTGCCTGTGACAGCTCTGCTGCTGCCTCTGGCTCTGCTGCTGCACGCCGCTAGACCCGATTATAAGGACGA
CGACGACAAGGAGCCCAAGAGCTGCGACAAGACACACACATGCCCTCTTctggccggCCTGGTGGCCGCCGACGCCG
TGGCCAGCCTGCTGATCGTGGGCGCCGTGTTCCTGTGCGCCAGGCCCAGGCGGAGCCCtGCCCAGGAGGACGGCAAG
GTGTACATCAACATGCCCGGCCGGGGCTACTTCCTGGGCAGGCTGGTGCCCAGGGGCAGGGGCGCTGCCGAGGCTGC
CACCCGGAAGCAGCGGATCACCGAGACCGAGAGCCCCTACCAGGAGCTGCAGGGCCAGCGGAGCGACGTGTACAGCG
ACCTGAACACCCAGAGGCCCTACTACAAG
SEQ ID NO: 73 (encoding polypeptide of SEQ ID NO: 63)
ATGGCTCTGCCTGTGACAGCTCTGCTGCTGCCTCTGGCTCTGCTGCTGCACGCCGCTAGACCCGATTATAAGGACGA
CGACGACAAGACCACAACACCTGCTCCTAGACCTCCCACCCCTGCTCCCACCATCGCCAGCCAGCCCCTGAGCCTGA
GACCCGAGGCCTGCAGACCCGCTGCTGGCGGCGCTGTGCATACCAGAGGCCTGGATTTCGCCTGCGACCTTctggcc
ggCCTGGTGGCCGCCGACGCCGTGGCCAGCCTGCTGATCGTGGGCGCCGTGTTCCTGTGCGCCAGGCCCAGGCGGAG
CCCtGCCCAGGAGGACGGCAAGGTGTACATCAACATGCCCGGCCGGGGCTACTTCCTGGGCAGGCTGGTGCCCAGGG
GCAGGGGCGCTGCCGAGGCTGCCACCCGGAAGCAGCGGATCACCGAGACCGAGAGCCCCTACCAGGAGCTGCAGGGC
CAGCGGAGCGACGTGTACAGCGACCTGAACACCCAGAGGCCCTACTACAAG
SEQ ID NO: 74 (encoding polypeptide of SEQ ID NO: 64/construct 1/N1012)
ATGATCCACCTGGGCCACATCCTGTTCCTGCTGCTGCTGCCCGTGGCCGCTGCCCAGACCACCCCTGGCGAGCGGAG
CAGCCTGCCTGCCTTCTACCCTGGCACCAGCGGCAGCTGCAGCGGCTGCGGCAGCCTGAGCCTGCCCCTGCTGGCCG
GCCTGGTGGCCGCCGACGCCGTGGCCAGCCTGCTGATCGTGGGCGCCGTGTTCCTGTGCGCCAGGCCCAGGCGGAGC
CCtGCCCAGGAGGACGGCAAGGTGTACATCAACATGCCCGGCCGGGGCTACTTCCTGGGCAGGCTGGTGCCCAGGGG
CAGGGGCGCTGCCGAGGCTGCCACCCGGAAGCAGCGGATCACCGAGACCGAGAGCCCCTACCAGGAGCTGCAGGGCC
AGCGGAGCGACGTGTACAGCGACCTGAACACCCAGAGGCCCTACTACAAGAGGCGGAAAAGGTCTGGGAGTGGGGCT
ACCAATTTCTCTCTCCTCAAGCAAGCCGGAGACGTTGAGGAAAACCCTGGaCCcATGGGCTGGATCCGGGGACGGAG
GAGCCGGCACAGCTGGGAGATGAGCGAGTTCCACAACTACAACCTGGACCTGAAGAAGAGCGACTTCAGCACCCGGT
GGCAGAAGCAGCGGTGCCCCGTGGTGAAGAGCAAGTGCCGGGAGAACGCCAGCCCCTTCTTCTTCTGCTGCTTCATC
GCCGTGGCtATGGGCATCCGGTTCATCATCATGGTGGCCATCTGGAGCGCCGTGTTCCTGAACAGCCTGTTCAACCA
GGAGGTGCAGATCCCCCTGACCGAGAGCTACTGCGGCCCCTGCCCCAAGAACTGGATCTGCTACAAGAACAACTGCT
ACCAGTTCTTCGACGAGAGCAAGAACTGGTACGAGAGCCAGGCCAGCTGCATGAGCCAGAACGCCAGCCTGCTGAAG
GTGTACAGCAAGGAGGACCAGGACCTGCTGAAGCTGGTGAAGAGCTACCACTGGATGGGCCTGGTGCACATCCCCAC
CAACGGCAGCTGGCAGTGGGAGGACGGCAGCATCCTGAGCCCCAACCTGCTGACCATCATCGAGATGCAGAAGGGCG
ACTGCGCCCTGTACGCCAGCAGCTTCAAGGGCTACATCGAGAACTGCAGCACCCCCAACACCTACATCTGCATGCAG
CGGACCGTG
SEQ ID NO: 75 (encoding polypeptide of SEQ ID NO: 65/construct 3)
ATGGCTCTGCCTGTGACAGCTCTGCTGCTGCCTCTGGCTCTGCTGCTGCACGCCGCTAGACCCGATTATAAGGACGA
CGACGACAAGCAGACCACCCCTGGCGAGCGGAGCAGCCTGCCTGCCTTCTACCCTGGCACCAGCGGCAGCTGCAGCG
GCTGCGGCAGCCTGAGCCTGCCCCTGCTGGCtGGCCTGGTGGCCGCCGACGCCGTGGCCAGCCTGCTGATCGTGGGC
GCCGTGTTCTACTTCCTGGGCAGGCTGGTGCCCAGGGGCAGGGGCGCTGCCGAGGCTGCCACCCGGAAGCAGCGGAT
CACCGAGACCGAGAGCCCCTACCAGGAGCTGCAGGGCCAGCGGAGCGACGTGTACAGCGACCTGAACACCCAGAGGC
CCTACTACAAGCGGAGAAAGCGCtccGGCTCCGGCGAGGGCcqcGGCAGCCTGCTGACCTGCGGCGACGTGGAAGAG
AACCCCGGACCCATGATCCACCTGGGCCACATCCTGTTCCTGCTGCTGCTGCCCGTGGCCGCTGCCCAAACAACACC
CGGCGAGAGATCCTCCTTGCCCGCTTTCTATCCCGGAACATCCGGAAGCTGTtccggaTGTGGATCCCTTTCTTTGC
ctttgCTTGCTGGATTGGTCGCAGCTGACGCTGTCGCTTCCCTCCTTATTGTCGGAGCTGTCTTCCTGTGCGCCAGG
CCCAGGCGGAGCCCtGCCCAGGAGGACGGCAAGGTGTACATCAACATGCCCGGCCGGGGCAGGCGGaagcgctccGG
GAGTGGGGCTACCAATTTCTCTCTCCTCAAGCAAGCCGGAGACGTTGAGGAAAACCCTGGaCCCATGGGCTGGATCC
GGGGACGGAGGAGCCGGCACAGCTGGGAGATGAGCGAGTTCCACAACTACAACCTGGACCTGAAGAAGAGCGACTTC
AGCACCCGGTGGCAGAAGCAGCGGTGCCCCGTGGTGAAGAGCAAGTGCCGGGAGAACGCCAGCCCCTTCTTCTTCTG
CTGCTTCATCGCCGTGGCtATGGGCATCCGGTTCATCATCATGGTGGCCATCTGGAGCGCCGTGTTCCTGAACAGCC
TGTTCAACCAGGAGGTGCAGATCCCCCTGACCGAGAGCTACTGCGGCCCCTGCCCCAAGAACTGGATCTGCTACAAG
AACAACTGCTACCAGTTCTTCGACGAGAGCAAGAACTGGTACGAGAGCCAGGCCAGCTGCATGAGCCAGAACGCCAG
CCTGCTGAAGGTGTACAGCAAGGAGGACCAGGACCTGCTGAAGCTGGTGAAGAGCTACCACTGGATGGGCCTGGTGC
ACATCCCCACCAACGGCAGCTGGCAGTGGGAGGACGGCAGCATCCTGAGCCCCAACCTGCTGACCATCATCGAGATG
CAGAAGGGCGACTGCGCCCTGTACGCCAGCAGCTTCAAGGGCTACATCGAGAACTGCAGCACCCCCAACACCTACAT
CTGCATGCAGCGGACCGTG
SEQ ID NO: 76 (encoding polypeptide of SEQ ID NO: 66/Construct 8)
ATGATCCACCTGGGCCACATCCTGTTCCTGCTGCTGCTGCCCGTGGCCGCTGCCCAGACCACCCCTGGCGAGCGGAG
CAGCCTGCCTGCCTTCTACCCTGGCACCAGCGGCAGCTGCAGCGGCTGCGGCAGCCTGAGCCTGCCCCTGCTGGCCG
GCCTGGTGGCCGCCGACGCCGTGGCCAGCCTGCTGATCGTGGGCGCCGTGTTCCTGTGCGCCAGGCCCAGGCGGAGC
CCtGCCCAGGAGGACGGCAAGGTGTACATCAACATGCCCGGCCGGGGCTACTTCCTGGGCAGGCTGGTGCCCAGGGG
CAGGGGCGCTGCCGAGGCTGCCACCCGGAAGCAGCGGATCACCGAGACCGAGAGCCCCTACCAGGAGCTGCAGGGCC
AGCGGAGCGACGTGTACAGCGACCTGAACACCCAGAGGCCCTACTACAAGAGGCGGAAAAGGTCTGGGAGTGGGGCT
ACCAATTTCTCTCTCCTCAAGCAAGCCGGAGACGTTGAGGAAAACCCTGGaCCCATGGGCTGGATCCGGGGACGGAG
GAGCCGGCACAGCTGGGAGATGAGCGAGTTCCACAACTACAACCTGGACCTGAAGAAGAGCGACTTCAGCACCCGGT
GGCAGAAGCAGCGGTGCCCCGTGGTGAAGAGCAAGTGCCGGGAGAACGCCAGCCCCTTCTTCTTCTGCTGCTTCATC
GCCGTGGCtATGGGCATCCGGTTCATCATCATGGTGGCCATCTGGAGCGCCGTGTTCCTGAACAGCCTGTTCAACCA
GGAGGTGCAGATCCCCCTGACCGAGAGCTACTGCGGCCCCTGCCCCAAGAACTGGATCTGCTACAAGAACAACTGCT
ACCAGTTCTTCGACGAGAGCAAGAACTGGTACGAGAGCCAGGCCAGCTGCATGAGCCAGAACGCCAGCCTGCTGAAG
GTGTACAGCAAGGAGGACCAGGACCTGCTGAAGCTGGTGAAGAGCTACCACTGGATGGGCCTGGTGCACATCCCCAC
CAACGGCAGCTGGCAGTGGGAGGACGGCAGCATCCTGAGCCCCAACCTGCTGACCATCATCGAGATGCAGAAGGGCG
ACTGCGCCCTGTACGCCAGCAGCTTCAAGGGCTACATCGAGAACTGCAGCACCCCCAACACCTACATCTGCATGCAG
CGGACCGTGAGAAGAAAGAGAAGCGGCAGCGGCGAGGGCAGAGGCAGCCTGCTGACCTGCGGCGACGTGGAGGAGAA
CCCCGGACccATGATTCATCTCGGACATATTCTCTTTCTCTTGCTCTTGCCTGTCGCTGCCGCTCAAACAACTCCCG
GAGAAAGATCTTCTCTCCCCGCTTTTTATCCCGGAACATCTGGATCTTGTTCTGGATGTGGATCTTTGTCTCTCCCT
CTCCTCGCTGGACTCGTCGCAGCTGATGCTGTCGCTTCTCTCTTGATTGTCGGAGCTGTCTTTTTGTGTAAGAGAGG
CAGAAAGAAGCTGCTGTACATCTTCAAGCAGCCCTTCATGAGACCCGTGCAGACCACCCAGGAGGAGGACGGCTGCA
GCTGCAGATTCCCCGAGGAGGAGGAGGGCGGCTGCGAGCTG
SEQ ID NO: 77 (encoding polypeptide of SEQ ID NO: 67/construct 9)
ATGATCCACCTGGGCCACATCCTGTTCCTGCTGCTGCTGCCCGTGGCCGCTGCCCAGACCACCCCTGGCGAGCGGAG
CAGCCTGCCTGCCTTCTACCCTGGCACCAGCGGCAGCTGCAGCGGCTGCGGCAGCCTGAGCCTGCCCCTGCTGGCCG
GCCTGGTGGCCGCCGACGCCGTGGCCAGCCTGCTGATCGTGGGCGCCGTGTTCCTGTGCGCCAGGCCCAGGCGGAGC
CCtGCCCAGGAGGACGGCAAGGTGTACATCAACATGCCCGGCCGGGGCTACTTCCTGGGCAGGCTGGTGCCCAGGGG
CAGGGGCGCTGCCGAGGCTGCCACCCGGAAGCAGCGGATCACCGAGACCGAGAGCCCCTACCAGGAGCTGCAGGGCC
AGCGGAGCGACGTGTACAGCGACCTGAACACCCAGAGGCCCTACTACAAGAGGCGGAAAAGGTCTGGGAGTGGGGCT
ACCAATTTCTCTCTCCTCAAGCAAGCCGGAGACGTTGAGGAAAACCCTGGaCCCATGGGCTGGATCCGGGGACGGAG
GAGCCGGCACAGCTGGGAGATGAGCGAGTTCCACAACTACAACCTGGACCTGAAGAAGAGCGACTTCAGCACCCGGT
GGCAGAAGCAGCGGTGCCCCGTGGTGAAGAGCAAGTGCCGGGAGAACGCCAGCCCCTTCTTCTTCTGCTGCTTCATC
GCCGTGGCtATGGGCATCCGGTTCATCATCATGGTGGCCATCTGGAGCGCCGTGTTCCTGAACAGCCTGTTCAACCA
GGAGGTGCAGATCCCCCTGACCGAGAGCTACTGCGGCCCCTGCCCCAAGAACTGGATCTGCTACAAGAACAACTGCT
ACCAGTTCTTCGACGAGAGCAAGAACTGGTACGAGAGCCAGGCCAGCTGCATGAGCCAGAACGCCAGCCTGCTGAAG
GTGTACAGCAAGGAGGACCAGGACCTGCTGAAGCTGGTGAAGAGCTACCACTGGATGGGCCTGGTGCACATCCCCAC
CAACGGCAGCTGGCAGTGGGAGGACGGCAGCATCCTGAGCCCCAACCTGCTGACCATCATCGAGATGCAGAAGGGCG
ACTGCGCCCTGTACGCCAGCAGCTTCAAGGGCTACATCGAGAACTGCAGCACCCCCAACACCTACATCTGCATGCAG
CGGACCGTGAGAAGAAAGAGAAGCGGCAGCGGCGAGGGCAGAGGCAGCCTGCTGACCTGCGGCGACGTGGAGGAGAA
CCCCGGACcTatggctctgcctgtgacagctctgctgctgcctctggctctgctgctgcacgccgctagacccgatt
ataaggacgacgacgacaagCAAACAACTCCCGGAGAAAGATCTTCTCTCCCCGCTTTTTATCCCGGAACATCTGGA
TCTTGTTCTGGATGTGGATCTTTGTCTCTCCCTCTCCTCGCTGGACTCGTCGCAGCTGATGCTGTCGCTTCTCTCTT
GATTGTCGGAGCTGTCTTTTTGTGTAAGAGAGGCAGAAAGAAGCTGCTGTACATCTTCAAGCAGCCCTTCATGAGAC
CCGTGCAGACCACCCAGGAGGAGGACGGCTGCAGCTGCAGATTCCCCGAGGAGGAGGAGGGCGGCTGCGAGCTG
SEQ ID NO: 78 (encoding polypeptide of SEQ ID NO: 68/construct 10)
ATGATCCACCTGGGCCACATCCTGTTCCTGCTGCTGCTGCCCGTGGCCGCTGCCCAGACCACCCCTGGCGAGCGGAG
CAGCCTGCCTGCCTTCTACCCTGGCACCAGCGGCAGCTGCAGCGGCTGCGGCAGCCTGAGCCTGCCCCTGCTGGCCG
GCCTGGTGGCCGCCGACGCCGTGGCCAGCCTGCTGATCGTGGGCGCCGTGTTCCTGTGCGCCAGGCCCAGGCGGAGC
CCtGCCCAGGAGGACGGCAAGGTGTACATCAACATGCCCGGCCGGGGCTACTTCCTGGGCAGGCTGGTGCCCAGGGG
CAGGGGCGCTGCCGAGGCTGCCACCCGGAAGCAGCGGATCACCGAGACCGAGAGCCCCTACCAGGAGCTGCAGGGCC
AGCGGAGCGACGTGTACAGCGACCTGAACACCCAGAGGCCCTACTACAAGAGGCGGAAAAGGTCTGGGAGTGGGGCT
ACCAATTTCTCTCTCCTCAAGCAAGCCGGAGACGTTGAGGAAAACCCTGGaCCcATGGGCTGGATCCGGGGACGGAG
GAGCCGGCACAGCTGGGAGATGAGCGAGTTCCACAACTACAACCTGGACCTGAAGAAGAGCGACTTCAGCACCCGGT
GGCAGAAGCAGCGGTGCCCCGTGGTGAAGAGCAAGTGCCGGGAGAACGCCAGCCCCTTCTTCTTCTGCTGCTTCATC
GCCGTGGCtATGGGCATCCGGTTCATCATCATGGTGGCCATCTGGAGCGCCGTGTTCCTGAACAGCCTGTTCAACCA
GGAGGTGCAGATCCCCCTGACCGAGAGCTACTGCGGCCCCTGCCCCAAGAACTGGATCTGCTACAAGAACAACTGCT
ACCAGTTCTTCGACGAGAGCAAGAACTGGTACGAGAGCCAGGCCAGCTGCATGAGCCAGAACGCCAGCCTGCTGAAG
GTGTACAGCAAGGAGGACCAGGACCTGCTGAAGCTGGTGAAGAGCTACCACTGGATGGGCCTGGTGCACATCCCCAC
CAACGGCAGCTGGCAGTGGGAGGACGGCAGCATCCTGAGCCCCAACCTGCTGACCATCATCGAGATGCAGAAGGGCG
ACTGCGCCCTGTACGCCAGCAGCTTCAAGGGCTACATCGAGAACTGCAGCACCCCCAACACCTACATCTGCATGCAG
CGGACCGTGAGAAGAAAGAGAAGCGGCAGCGGCGAGGGCAGAGGCAGCCTGCTGACCTGCGGCGACGTGGAGGAGAA
CCCCGGACccATGATTCATCTCGGACATATTCTCTTTCTCTTGCTCTTGCCTGTCGCTGCCGCTCAAACAACTCCCG
GAGAAAGATCTTCTCTCCCCGCTTTTTATCCCGGAACATCTGGATCTTGTTCTGGATGTGGATCTTTGTCTCTCCCT
CTCCTCGCTGGACTCGTCGCAGCTGATGCTGTCGCTTCTCTCTTGATTGTCGGAGCTGTCTTTTTGTGTCAGAGGCG
GAAGTACCGGAGCAACAAGGGCGAGAGCCCCGTGGAGCCTGCCGAGCCCTGCCACTACAGCTGTCCCCGGGAGGAGG
AGGGCAGCACCATCCCCATCCAGGAGGACTACCGGAAGCCCGAGCCTGCCTGCAGCCCC
SEQ ID NO: 79 (encoding polypeptide of SEQ ID NO: 69/Construct 11)
ATGATCCACCTGGGCCACATCCTGTTCCTGCTGCTGCTGCCCGTGGCCGCTGCCCAGACCACCCCTGGCGAGCGGAG
CAGCCTGCCTGCCTTCTACCCTGGCACCAGCGGCAGCTGCAGCGGCTGCGGCAGCCTGAGCCTGCCCCTGCTGGCCG
GCCTGGTGGCCGCCGACGCCGTGGCCAGCCTGCTGATCGTGGGCGCCGTGTTCCTGTGCGCCAGGCCCAGGCGGAGC
CCtGCCCAGGAGGACGGCAAGGTGTACATCAACATGCCCGGCCGGGGCTACTTCCTGGGCAGGCTGGTGCCCAGGGG
CAGGGGCGCTGCCGAGGCTGCCACCCGGAAGCAGCGGATCACCGAGACCGAGAGCCCCTACCAGGAGCTGCAGGGCC
AGCGGAGCGACGTGTACAGCGACCTGAACACCCAGAGGCCCTACTACAAGAGGCGGAAAAGGTCTGGGAGTGGGGCT
ACCAATTTCTCTCTCCTCAAGCAAGCCGGAGACGTTGAGGAAAACCCTGGaCCcATGGGCTGGATCCGGGGACGGAG
GAGCCGGCACAGCTGGGAGATGAGCGAGTTCCACAACTACAACCTGGACCTGAAGAAGAGCGACTTCAGCACCCGGT
GGCAGAAGCAGCGGTGCCCCGTGGTGAAGAGCAAGTGCCGGGAGAACGCCAGCCCCTTCTTCTTCTGCTGCTTCATC
GCCGTGGCtATGGGCATCCGGTTCATCATCATGGTGGCCATCTGGAGCGCCGTGTTCCTGAACAGCCTGTTCAACCA
GGAGGTGCAGATCCCCCTGACCGAGAGCTACTGCGGCCCCTGCCCCAAGAACTGGATCTGCTACAAGAACAACTGCT
ACCAGTTCTTCGACGAGAGCAAGAACTGGTACGAGAGCCAGGCCAGCTGCATGAGCCAGAACGCCAGCCTGCTGAAG
GTGTACAGCAAGGAGGACCAGGACCTGCTGAAGCTGGTGAAGAGCTACCACTGGATGGGCCTGGTGCACATCCCCAC
CAACGGCAGCTGGCAGTGGGAGGACGGCAGCATCCTGAGCCCCAACCTGCTGACCATCATCGAGATGCAGAAGGGCG
ACTGCGCCCTGTACGCCAGCAGCTTCAAGGGCTACATCGAGAACTGCAGCACCCCCAACACCTACATCTGCATGCAG
CGGACCGTGAGAAGAAAGAGAAGCGGCAGCGGCGAGGGCAGAGGCAGCCTGCTGACCTGCGGCGACGTGGAGGAGAA
CCCCGGACcTatggctctgcctgtgacagctctgctgctgcctctggctctgctgctgcacgccgctagacccgatt
ataaggacgacgacgacaagCAAACAACTCCCGGAGAAAGATCTTCTCTCCCCGCTTTTTATCCCGGAACATCTGGA
TCTTGTTCTGGATGTGGATCTTTGTCTCTCCCTCTCCTCGCTGGACTCGTCGCAGCTGATGCTGTCGCTTCTCTCTT
GATTGTCGGAGCTGTCTTTTTGTGTCAGAGGCGGAAGTACCGGAGCAACAAGGGCGAGAGCCCCGTGGAGCCTGCCG
AGCCCTGCCACTACAGCTGTCCCCGGGAGGAGGAGGGCAGCACCATCCCCATCCAGGAGGACTACCGGAAGCCCGAG
CCTGCCTGCAGCCCC
SEQ ID NO: 80 (A20FMDV2 peptide)
NAVPNLRGDLQVLAQKVART
SEQ ID NO: 81 (CD124 signal peptide)
MGWLCSGLLFPVSCLVLLQVASSGN
SEQ ID NO: 82 (CD28 aa114-220)
IEVMYPPPYLDNEKSNGTIIHVKGKHLCPSPLFPGPSKPFWVLVVVGGVLACYSLLVTVAFIIFWVRSKRSRLLHSD
YMNMTPRRPGPTRKHYQPYAPPRDFAAYRS
SEQ ID NO: 83 (CD247 aa52-164)
RVKFSRSADAPAYQQGQNQLYNELNLGRREEYDVLDKRRGRDPEMGGKPRRKNPQEGLYNELQKDKMAEAYSEIGMK
GERRRGKGHDGLYQGLSTATKDTYDALHMQALPPR
SEQ ID NO: 84 (SEQ ID NO: 1 of WO 2019/182425)
MGWSCIILFLVATATGVHSQIQLVQSGPELKKPGETVKISCKTSGYTFTDYSMHWVNQAP
GKGLKWMGWINTETGEPTYTDDFKGRFAFSLETSASTAYLQINNLKNEDTATYFCARTAV
YWGQGTTLTVSSGSTSGSGKPGSGEGSDIQMTQSPSSLSASLGERVSLTCRASQEISGSL
SWLQQKPDGTIKRLIYAASTLNSGVPKRFSGRRSGSDYSLTISSLESEDFVDYYCLQYSS
YPWSFGGGTKLEIKEPKSPDKTHTCPPCPSHTQPLGVFLFPPKPKDQLMISRTPEVTCVV
VDVSQEDPEVQFNWYVDGVEVHNAKTKPREEQFNSTYRVVSVLTVLHQDWLNGKEYKCKV
SNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVLHEALHNHYTQKSLSL
SLGKFWVLVVVGGVLACYSLLVTVAFIIFWVARPRRSPAQEDGKVYINMPGRGGRLVPRG
RGAAEAATRKQRITETESPYQELQGQRSDVYSDLNTQRPYYKRVKFSRSADAPAYQQGQN
QLYNELNLGRREEYDVLDKRRGRDPEMGGKPQRRKNPQEGLYNELQKDKMAEAYSEIGMK
GERRRGKGHDGLYQGLSTATKDTYDALHMQALPPR
SEQ ID NO: 85 (SEQ ID NO: 9 of WO 2019/182425)
ARPRRSPAQEDGKVYINMPGRG
SEQ ID NO: 86 (SEQ ID NO: 11 of WO 2019/182425)
GRLVPRGRGAAEAATRKQRITETESPYQELQGQRSDVYSDLNTQRPYYK
SEQ ID NO: 87 (sequence of human CXCR2 polypeptide expressed in N1012_CXCR2)
MEDENMESDSFEDFWKGEDLSNYSYSSTLPPFLLDAAPCEPESLEINKYFVVIIYALVFLLSLLGNSLVMLVILYSR
VGRSVTDVYLLNLALADLLFALTLPIWAASKVNGWIFGTFLCKVVSLLKEVNFYSGILLLACISVDRYLAIVHATRT
LTQKRYLVKFICLSIWGLSLLLALPVLLFRRTVYSSNVSPACYEDMGNNTANWRMLLRILPQSFGFIVPLLIMLFCY
GFTLRTLFKAHMGQKHRAMRVIFAVVLIFLLCWLPYNLVLLADTLMRTQVIQETCERRNHIDRALDATEILGILHSC
LNPLIYAFIGQKFRHGLLKILAIHGLISKDSLPKDSRPSFVGSSSGHTSTTL
SEQ ID NO: 88 (nucleic acid sequence encoding polypeptide of SEQ ID NO: 87)
atggaggatttcaatatggagagcgactccttcgaggatttttggaagggcgaggacctgtctaactacagctatag
ctccacactgcccccttttctgctggatgccgccccttgtgagccagagtccctggagatcaacaagtacttcgtgg
tcatcatctatgccctggtgtttctgctgtctctgctgggcaatagcctggtcatgctggtcatcctgtactccagg
gtgggccgctctgtgaccgacgtgtatctgctgaatctggccctggccgatctgctgttcgcactgacactgccaat
ctgggcagcaagcaaggtgaacggctggatcttcggcacctttctgtgcaaggtggtgtctctgctgaaggaggtga
acttctacagcggcatcctgctgctggcctgtatctccgtggaccggtatctggccatcgtgcacgccaccaggaca
ctgacccagaagcggtacctggtgaagttcatctgcctgagcatctggggactgtccctgctgctggccctgcctgt
gctgctgtttaggcgcacagtgtactctagcaacgtgtctccagcctgttatgaggatatgggcaacaataccgcca
attggaggatgctgctgcgcatcctgccacagagcttcggctttatcgtgcccctgctgatcatgctgttctgctac
ggctttacactgcggaccctgttcaaggcccacatgggccagaagcaccgggccatgagagtgatcttcgccgtggt
gctgatctttctgctgtgctggctgccctataacctggtgctgctggccgacacactgatgcggacccaggtcatcc
aggagacatgcgagcggagaaaccacatcgacagagccctggatgccaccgagatcctgggcatcctgcactcctgt
ctgaatcctctgatctatgccttcatcggccagaagtttaggcacggcctgctgaagatcctggccatccacggcct
gatctccaaggactctctgcccaaggatagccgcccttccttcgtgggctcctctagcggccacacctctaccacac
tg
SEQ ID NO: 89 (nucleic acid sequence of SFG N1012_CXCR2)
gatccggattagtccaatttgttaaagacaggatatcagtggtccaggctctagttttgactcaacaatatcaccag
ctgaagcctatagagtacgagccatagataaaataaaagattttatttagtctccagaaaaaggggggaatgaaaga
ccccacctgtaggtttggcaagctagcttaagtaacgccattttgcaaggcatggaaaaatacataactgagaatag
agaagttcagatcaaggtcaggaacagatggaacagctgaatatgggccaaacaggatatctgtggtaagcagttcc
tgccccggctcagggccaagaacagatggaacagctgaatatgggccaaacaggatatctgtggtaagcagttcctg
ccccggctcagggccaagaacagatggtccccagatgcggtccagccctcagcagtttctagagaaccatcagatgt
ttccagggtgccccaaggacctgaaatgaccctgtgccttatttgaactaaccaatcagttcgcttctcgcttctgt
tcgcgcgcttctgctccccgagctcaataaaagagcccacaacccctcactcggggcgccagtcctccgattgactg
agtcgcccgggtacccgtgtatccaataaaccctcttgcagttgcatccgacttgtggtctcgctgttccttgggag
ggtctcctctgagtgattgactacccgtcagcgggggtctttcacatgcagcatgtatcaaaattaatttggttttt
tttcttaagtatttacattaaatggccatagtacttaaagttacattggcttccttgaaataaacatggagtattca
gaatgtgtcataaatatttctaattttaagatagtatctccattggctttctactttttcttttatttttttttgtc
ctctgtcttccatttgttgttgttgttgtttgtttgtttgtttgttggttggttggttaatttttttttaaagatcc
tacactatagttcaagctagactattagctactctgtaacccagggtgaccttgaagtcatgggtagcctgctgttt
tagccttcccacatctaagattacaggtatgagctatcatttttggtatattgattgattgattgattgatgtgtgt
gtgtgtgattgtgtttgtgtgtgtgattgtgtatatgtgtgtatggttgtgtgtgattgtgtgtatgtatgtttgtg
tgtgattgtgtgtgtgtgattgtgcatgtgtgtgtgtgtgattgtgtttatgtgtatgattgtgtgtgtgtgtgtgt
gtgtgtgtgtgtgtgtgtgtgtgtgtgtgttgtgtatatatatttatggtagtgagaggcaacgctccggctcaggt
gtcaggttggtttttgagacagagtctttcacttagcttggaattcactggccgtcgttttacaacgtcgtgactgg
gaaaaccctggcgttacccaacttaatcgccttgcagcacatccccctttcgccagctggcgtaatagcgaagaggc
ccgcaccgatcgcccttcccaacagttgcgcagcctgaatggcgaatggcgcctgatgcggtattttctccttacgc
atctgtgcggtatttcacaccgcatatggtgcactctcagtacaatctgctctgatgccgcatagttaagccagccc
cgacacccgccaacacccgctgacgcgccctgacgggcttgtctgctcccggcatccgcttacagacaagctgtgac
cgtctccgggagctgcatgtgtcagaggttttcaccgtcatcaccgaaacgcgcgagacgaaagggcctcgtgatac
gcctatttttataggttaatgtcatgataataatggtttcttagacgtcaggtggcacttttcggggaaatgtgcgc
ggaacccctatttgtttatttttctaaatacattcaaatatgtatccgctcatgagacaataaccctgataaatgct
tcaataatattgaaaaaggaagagtatgagtattcaacatttccgtgtcgcccttattcccttttttgcggcatttt
gccttcctgtttttgctcacccagaaacgctggtgaaagtaaaagatgctgaagatcagttgggtgcacgagtgggt
tacatcgaactggatctcaacagcggtaagatccttgagagttttcgccccgaagaacgttttccaatgatgagcac
ttttaaagttctgctatgtggcgcggtattatcccgtattgacgccgggcaagagcaactcggtcgccgcatacact
attctcagaatgacttggttgagtactcaccagtcacagaaaagcatcttacggatggcatgacagtaagagaatta
tgcagtgctgccataaccatgagtgataacactgcggccaacttacttctgacaacgatcggaggaccgaaggagct
aaccgcttttttgcacaacatgggggatcatgtaactcgccttgatcgttgggaaccggagctgaatgaagccatac
caaacgacgagcgtgacaccacgatgcctgtagcaatggcaacaacgttgcgcaaactattaactggcgaactactt
actctagcttcccggcaacaattaatagactggatggaggcggataaagttgcaggaccacttctgcgctcggccct
tccggctggctggtttattgctgataaatctggagccggtgagcgtgggtctcgcggtatcattgcagcactggggc
cagatggtaagccctcccgtatcgtagttatctacacgacggggagtcaggcaactatggatgaacgaaatagacag
atcgctgagataggtgcctcactgattaagcattggtaactgtcagaccaagtttactcatatatactttagattga
tttaaaacttcatttttaatttaaaaggatctaggtgaagatcctttttgataatctcatgaccaaaatcccttaac
gtgagttttcgttccactgagcgtcagaccccgtagaaaagatcaaaggatcttcttgagatcctttttttctgcgc
gtaatctgctgcttgcaaacaaaaaaaccaccgctaccagcggtggtttgtttgccggatcaagagctaccaactct
ttttccgaaggtaactggcttcagcagagcgcagataccaaatactgttcttctagtgtagccgtagttaggccacc
acttcaagaactctgtagcaccgcctacatacctcgctctgctaatcctgttaccagtggctgctgccagtggcgat
aagtcgtgtcttaccgggttggactcaagacgatagttaccggataaggcgcagcggtcgggctgaacggggggttc
gtgcacacagcccagcttggagcgaacgacctacaccgaactgagatacctacagcgtgagctatgagaaagcgcca
cgcttcccgaagggagaaaggcggacaggtatccggtaagcggcagggtcggaacaggagagcgcacgagggagctt
ccagggggaaacgcctggtatctttatagtcctgtcgggtttcgccacctctgacttgagcgtcgatttttgtgatg
ctcgtcaggggggcggagcctatggaaaaacgccagcaacgcggcctttttacggttcctggccttttgctggcctt
ttgctcacatgttctttcctgcgttatcccctgattctgtggataaccgtattaccgcctttgagtgagctgatacc
gctcgccgcagccgaacgaccgagcgcagcgagtcagtgagcgaggaagcggaagagcgcccaatacgcaaaccgcc
tctccccgcgcgttggccgattcattaatgcagctggcacgacaggtttcccgactggaaagcgggcagtgagcgca
acgcaattaatgtgagttagctcactcattaggcaccccaggctttacactttatgcttccggctcgtatgttgtgt
ggaattgtgagcggataacaatttcacacaggaaacagctatgaccatgattacgccaagctttgctcttaggagtt
tcctaatacatcccaaactcaaatatataaagcatttgacttgttctatgccctagggggcggggggaagctaagcc
agctttttttaacatttaaaatgttaattccattttaaatgcacagatgtttttatttcataagggtttcaatgtgc
atgaatgctgcaatattcctgttaccaaagctagtataaataaaaatagataaacgtggaaattacttagagtttct
gtcattaacgtttccttcctcagttgacaacataaatgcgctgctgagaagccagtttgcatctgtcaggatcaatt
tcccattatgccagtcatattaattactagtcaattagttgatttttatttttgacatatacatgtgaaagacccca
cctgtaggtttggcaagctagcttaagtaacgccattttgcaaggcatggaaaaatacataactgagaatagaaaag
ttcagatcaaggtcaggaacagatggaacagctgaatatgggccaaacaggatatctgtggtaagcagttcctgccc
cggctcagggccaagaacagatggaacagctgaatatgggccaaacaggatatctgtggtaagcagttcctgccccg
gctcagggccaagaacagatggtccccagatgcggtccagccctcagcagtttctagagaaccatcagatgtttcca
gggtgccccaaggacctgaaatgaccctgtgccttatttgaactaaccaatcagttcgcttctcgcttctgttcgcg
cgcttctgctccccgagctcaataaaagagcccacaacccctcactcggcgcgccagtcctccgattgactgagtcg
cccgggtacccgtgtatccaataaaccctcttgcagttgcatccgacttgtggtctcgctgttccttgggagggtct
cctctgagtgattgactacccgtcagcgggggtctttcatttgggggctcgtccgggatcgggagacccctgcccag
ggaccaccgacccaccaccgggaggtaagctggccagcaacttatctgtgtctgtccgattgtctagtgtctatgac
tgattttatgcgcctgcgtcggtactagttagctaactagctctgtatctggcggacccgtggtggaactgacgagt
tcggaacacccggccgcaaccctgggagacgtcccagggacttcgggggccgtttttgtggcccgacctgagtccta
aaatcccgatcgtttaggactctttggtgcaccccccttagaggagggatatgtggttctggtaggagacgagaacc
taaaacagttcccgcctccgtctgaatttttgctttcggtttgggaccgaagccgcgccgcgcgtcttgtctgctgc
agcatcgttctgtgttgtctctgtctgactgtgtttctgtatttgtctgaaaatatgggcccgggctagactgttac
cactcccttaagtttgaccttaggtcactggaaagatgtcgagcggatcgctcacaaccagtcggtagatgtcaaga
agagacgttgggttaccttctgctctgcagaatggccaacctttaacgtcggatggccgcgagacggcacctttaac
cgagacctcatcacccaggttaagatcaaggtcttttcacctggcccgcatggacacccagaccaggtcccctacat
cgtgacctgggaagccttggcttttgacccccctccctgggtcaagccctttgtacaccctaagcctccgcctcctc
ttcctccatccgccccgtctctcccccttgaacctcctcgttcgaccccgcctcgatcctccctttatccagccctc
actccttctctaggcgcccccatatggccatatgagatcttatatggggcacccccgccccttgtaaacttccctga
ccctgacatgacaagagttactaacagcccctctctccaagctcacttacaggctctctacttagtccagcacgaag
tctggagacctctggcggcagcctaccaagaacaactggaccgaccggtggtacctcacccttaccgagtcggcgac
acagtgtgggtccgccgacaccagactaagaacctagaacctcgctggaaaggaccttacacagtcctgctgaccac
ccccaccgccctcaaagtagacggcatcgcagcttggatacacgccgcccacgtgaaggctgccgaccccgggggtg
gaccatcctctagactgccatgatccacctgggccacatcctgttcctgctgctgctgcccgtggccgctgcccaga
ccacccctggcgagcggagcagcctgcctgccttctaccctggcaccagcggcagctgcagcggctgcggcagcctg
agcctgcccctgctggccggcctggtggccgccgacgccgtggccagcctgctgatcgtgggcgccgtgttcctgtg
cgccaggcccaggcggagccctgcccaggaggacggcaaggtgtacatcaacatgcccggccggggctacttcctgg
gcaggctggtgcccaggggcaggggcgctgccgaggctgccacccggaagcagcggatcaccgagaccgagagcccc
taccaggagctgcagggccagcggagcgacgtgtacagcgacctgaacacccagaggccctactacaagaggcggaa
aaggtctgggagtggggctaccaatttctctctcctcaagcaagccggagacgttgaggaaaaccctggacccatgg
gctggatccggggacggaggagccggcacagctgggagatgagcgagttccacaactacaacctggacctgaagaag
agcgacttcagcacccggtggcagaagcagcggtgccccgtggtgaagagcaagtgccgggagaacgccagcccctt
cttcttctgctgcttcatcgccgtggctatgggcatccggttcatcatcatggtggccatctggagcgccgtgttcc
tgaacagcctgttcaaccaggaggtgcagatccccctgaccgagagctactgcggcccctgccccaagaactggatc
tgctacaagaacaactgctaccagttcttcgacgagagcaagaactggtacgagagccaggccagctgcatgagcca
gaacgccagcctgctgaaggtgtacagcaaggaggaccaggacctgctgaagctggtgaagagctaccactggatgg
gcctggtgcacatccccaccaacggcagctggcagtgggaggacggcagcatcctgagccccaacctgctgaccatc
atcgagatgcagaagggcgactgcgccctgtacgccagcagcttcaagggctacatcgagaactgcagcacccccaa
cacctacatctgcatgcagcggacagtgcggagaaagagatccggatctggagagggaagaggaagcctgctgacct
gcggcgacgtggaggagaacccaggacccatggaggatttcaatatggagagcgactccttcgaggatttttggaag
ggcgaggacctgtctaactacagctatagctccacactgcccccttttctgctggatgccgccccttgtgagccaga
gtccctggagatcaacaagtacttcgtggtcatcatctatgccctggtgtttctgctgtctctgctgggcaatagcc
tggtcatgctggtcatcctgtactccagggtgggccgctctgtgaccgacgtgtatctgctgaatctggccctggcc
gatctgctgttcgcactgacactgccaatctgggcagcaagcaaggtgaacggctggatcttcggcacctttctgtg
caaggtggtgtctctgctgaaggaggtgaacttctacagcggcatcctgctgctggcctgtatctccgtggaccggt
atctggccatcgtgcacgccaccaggacactgacccagaagcggtacctggtgaagttcatctgcctgagcatctgg
ggactgtccctgctgctggccctgcctgtgctgctgtttaggcgcacagtgtactctagcaacgtgtctccagcctg
ttatgaggatatgggcaacaataccgccaattggaggatgctgctgcgcatcctgccacagagcttcggctttatcg
tgcccctgctgatcatgctgttctgctacggctttacactgcggaccctgttcaaggcccacatgggccagaagcac
cgggccatgagagtgatcttcgccgtggtgctgatctttctgctgtgctggctgccctataacctggtgctgctggc
cgacacactgatgcggacccaggtcatccaggagacatgcgagcggagaaaccacatcgacagagccctggatgcca
ccgagatcctgggcatcctgcactcctgtctgaatcctctgatctatgccttcatcggccagaagtttaggcacggc
ctgctgaagatcctggccatccacggcctgatctccaaggactctctgcccaaggatagccgcccttccttcgtggg
ctcctctagcggccacacctctaccacactgtgacagccactcgag
SEQ ID NO: 90 (protein encoded by SEQ ID NO: 89 (includes: (i) fusion of full length
DAP10/DAP12 intracellular domain; (ii) furin cleavage site (RRKR); (iii) SGSG linker;
(iv) P2A skip sequence; (v) NKG2D; (vi) furin cleavage site (RRKR); (vii) SGSG linker;
(viii) T2A skip sequence; (ix) CXCR2)
MIHLGHILFLLLLPVAAAQTTPGERSSLPAFYPGTSGSCSGCGSLSLPLLAGLVAADAVASLLIVGAVELCARPRRS
PAQEDGKVYINMPGRGYFLGRLVPRGRGAAEAATRKQRITETESPYQELQGQRSDVYSDLNTQRPYYKRRKRSGSGA
TNFSLLKQAGDVEENPGPMGWIRGRRSRHSWEMSEFHNYNLDLKKSDFSTRWQKQRCPVVKSKCRENASPFFFCCFI
AVAMGIRFIIMVAIWSAVELNSLFNQEVQIPLTESYCGPCPKNWICYKNNCYQFFDESKNWYESQASCMSQNASLLK
VYSKEDQDLLKLVKSYHWMGLVHIPTNGSWQWEDGSILSPNLLTIIEMQKGDCALYASSFKGYIENCSTPNTYICMQ
RTVRRKRSGSGEGRGSLLTCGDVEENPGPMEDFNMESDSFEDFWKGEDLSNYSYSSTLPPFLLDAAPCEPESLEINK
YFVVIIYALVFLLSLLGNSLVMLVILYSRVGRSVTDVYLLNLALADLLFALTLPIWAASKVNGWIFGTFLCKVVSLL
KEVNFYSGILLLACISVDRYLAIVHATRTLTQKRYLVKFICLSIWGLSLLLALPVLLFRRTVYSSNVSPACYEDMGN
NTANWRMLLRILPQSFGFIVPLLIMLFCYGFTLRTLFKAHMGQKHRAMRVIFAVVLIFLLCWLPYNLVLLADTLMRT
QVIQETCERRNHIDRALDATEILGILHSCLNPLIYAFIGQKFRHGLLKILAIHGLISKDSLPKDSRPSFVGSSSGHT
STTL
SEQ ID NO: 91 (encoding the polypeptide of SEQ ID NO: 90)
ATGATCCACCTGGGCCACATCCTGTTCCTGCTGCTGCTGCCCGTGGCCGCTGCCCAG
ACCACCCCTGGCGAGCGGAGCAGCCTGCCTGCCTTCTACCCTGGCACCAGCGGCAGCTGCAGCGGCTGCGGCAGCCTGAG
CCTGCCCCTGCTGGCCGGCCTGGTGGCCGCCGACGCCGTGGCCAGCCTGCTGATCGTGGGCGCCGTGTTCCTGTGCGCCA
GGCCCAGGCGGAGCCCtGCCCAGGAGGACGGCAAGGTGTACATCAACATGCCCGGCCGGGGCTACTTCCTGGGCAGGCTG
GTGCCCAGGGGCAGGGGCGCTGCCGAGGCTGCCACCCGGAAGCAGCGGATCACCGAGACCGAGAGCCCCTACCAGGAGCT
GCAGGGCCAGCGGAGCGACGTGTACAGCGACCTGAACACCCAGAGGCCCTACTACAAGAGGCGGAAAAGGTCTGGGAGTG
GGGCTACCAATTTCTCTCTCCTCAAGCAAGCCGGAGACGTTGAGGAAAACCCTGGaCCcATGGGCTGGATCCGGGGACGG
AGGAGCCGGCACAGCTGGGAGATGAGCGAGTTCCACAACTACAACCTGGACCTGAAGAAGAGCGACTTCAGCACCCGGTG
GCAGAAGCAGCGGTGCCCCGTGGTGAAGAGCAAGTGCCGGGAGAACGCCAGCCCCTTCTTCTTCTGCTGCTTCATCGCCG
TGGCtATGGGCATCCGGTTCATCATCATGGTGGCCATCTGGAGCGCCGTGTTCCTGAACAGCCTGTTCAACCAGGAGGTG
CAGATCCCCCTGACCGAGAGCTACTGCGGCCCCTGCCCCAAGAACTGGATCTGCTACAAGAACAACTGCTACCAGTTCTT
CGACGAGAGCAAGAACTGGTACGAGAGCCAGGCCAGCTGCATGAGCCAGAACGCCAGCCTGCTGAAGGTGTACAGCAAGG
AGGACCAGGACCTGCTGAAGCTGGTGAAGAGCTACCACTGGATGGGCCTGGTGCACATCCCCACCAACGGCAGCTGGCAG
TGGGAGGACGGCAGCATCCTGAGCCCCAACCTGCTGACCATCATCGAGATGCAGAAGGGCGACTGCGCCCTGTACGCCAG
CAGCTTCAAGGGCTACATCGAGAACTGCAGCACCCCCAACACCTACATCTGCATGCAGCGGACAGTGCGGAGAAAGAGAT
CCGGATCTGGAGAGGGAAGAGGAAGCCTGCTGACCTGCGGCGACGTGGAGGAGAACCCAGGACCCATGGAGGATTTCAAT
ATGGAGAGCGACTCCTTCGAGGATTTTTGGAAGGGCGAGGACCTGTCTAACTACAGCTATAGCTCCACACTGCCCCCTTT
TCTGCTGGATGCCGCCCCTTGTGAGCCAGAGTCCCTGGAGATCAACAAGTACTTCGTGGTCATCATCTATGCCCTGGTGT
TTCTGCTGTCTCTGCTGGGCAATAGCCTGGTCATGCTGGTCATCCTGTACTCCAGGGTGGGCCGCTCTGTGACCGACGTG
TATCTGCTGAATCTGGCCCTGGCCGATCTGCTGTTCGCACTGACACTGCCAATCTGGGCAGCAAGCAAGGTGAACGGCTG
GATCTTCGGCACCTTTCTGTGCAAGGTGGTGTCTCTGCTGAAGGAGGTGAACTTCTACAGCGGCATCCTGCTGCTGGCCT
GTATCTCCGTGGACCGGTATCTGGCCATCGTGCACGCCACCAGGACACTGACCCAGAAGCGGTACCTGGTGAAGTTCATC
TGCCTGAGCATCTGGGGACTGTCCCTGCTGCTGGCCCTGCCTGTGCTGCTGTTTAGGCGCACAGTGTACTCTAGCAACGT
GTCTCCAGCCTGTTATGAGGATATGGGCAACAATACCGCCAATTGGAGGATGCTGCTGCGCATCCTGCCACAGAGCTTCG
GCTTTATCGTGCCCCTGCTGATCATGCTGTTCTGCTACGGCTTTACACTGCGGACCCTGTTCAAGGCCCACATGGGCCAG
AAGCACCGGGCCATGAGAGTGATCTTCGCCGTGGTGCTGATCTTTCTGCTGTGCTGGCTGCCCTATAACCTGGTGCTGCT
GGCCGACACACTGATGCGGACCCAGGTCATCCAGGAGACATGCGAGCGGAGAAACCACATCGACAGAGCCCTGGATGCCA
CCGAGATCCTGGGCATCCTGCACTCCTGTCTGAATCCTCTGATCTATGCCTTCATCGGCCAGAAGTTTAGGCACGGCCTG
CTGAAGATCCTGGCCATCCACGGCCTGATCTCCAAGGACTCTCTGCCCAAGGATAGCCGCCCTTCCTTCGTGGGCTCCTC
TAGCGGCCACACCTCTACCACACTGTGACAGCCACTCGAG
SEQ ID NO: 92 (amino acid sequence of a replica of the Cyad-01 CAR (NKG2D fused to the
intracellular domain of CD3ζ)
MRVKFSRSADAPAYQQGQNQLYNELNLGRREEYDVLDKRRGRDPEMGGKPRRKNPQEGLYNELQKDKMAEAYSEIGM
KGERRRGKGHDGLYQGLSTATKDTYDALHMQALPPRMGWIRGRRSRHSWEMSEFHNYNLDLKKSDFSTRWQKQRCPV
VKSKCRENASPFFFCCFIAVAMGIRFIIMVAIWSAVELNSLFNQEVQIPLTESYCGPCPKNWICYKNNCYQFFDESK
NWYESQASCMSQNASLLKVYSKEDQDLLKLVKSYHWMGLVHIPTNGSWQWEDGSILSPNLLTIIEMQKGDCALYASS
FKGYIENCSTPNTYICMQRTV
SEQ ID NO: 93 (nucleic acid sequence encoding polypeptide of SEQ ID NO: 92)
ATGAGAGTGAAGTTCAGCAGGAGCGCAGACGCCCCcGCGTACCAGCAGGGCCAGAACCAGCTCTATAACGAGCTCAA
TCTAGGACGAAGAGAGGAGTACGATGTTTTGGACAAGAGACGTGGCCGGGACCCTGAGATGGGGGGAAAGCCGAGAA
GGAAGAACCCTCAGGAAGGCCTGTACAATGAACTGCAGAAAGATAAGATGGCGGAGGCCTACAGTGAGATTGGGATG
AAAGGCGAGCGCCGGAGGGGCAAGGGGCACGATGGCCTTTACCAGGGTCTCAGTACAGCCACCAAGGACACCTACGA
TGCATTGCACATGCAGGCCCTGCCCCCTCGCATGGGCTGGATCCGCGGCCGCAGGAGCCGGCACAGCTGGGAGATGA
GCGAGTTCCACAACTACAACCTGGACCTGAAGAAGAGCGACTTCAGCACCCGGTGGCAGAAGCAGCGGTGCCCCGTG
GTGAAGAGCAAGTGCCGGGAGAACGCCAGCCCCTTCTTCTTCTGCTGCTTCATCGCCGTGGCtATGGGCATCCGGTT
TATAATCATGGTGGCCATCTGGAGCGCCGTGTTCCTGAACAGCCTGTTCAACCAGGAGGTGCAGATCCCCCTGACCG
AGAGCTACTGCGGCCCCTGCCCCAAGAACTGGATCTGCTACAAGAACAACTGCTACCAGTTCTTCGACGAGAGCAAG
AACTGGTACGAGAGCCAGGCCAGCTGCATGAGCCAGAACGCCAGCCTGCTGAAGGTGTACAGCAAGGAGGACCAGGA
CCTGCTGAAGCTGGTGAAGAGCTACCACTGGATGGGCCTGGTGCACATCCCCACCAACGGCAGCTGGCAGTGGGAGG
ACGGCAGCATCCTGAGCCCCAACCTGCTGACCATCATCGAGATGCAGAAGGGCGACTGCGCCCTGTACGCCAGCAGC
TTCAAGGGCTACATCGAGAACTGCAGCACCCCCAACACCTACATCTGCATGCAGCGGACCGTGtaa
SEQ ID NO: 94 (nucleic acid sequence encoding CYAD-01_10-polypeptides of SEQ ID NO:
92 and SEQ ID NO: 1)
ATGATCCACCTGGGCCACATCCTGTTCCTGCTGCTGCTGCCCGTGGCCGCTGCCCAGACCACCCCTGGCGAGCGGAG
CAGCCTGCCTGCCTTCTACCCTGGCACCAGCGGCAGCTGCAGCGGCTGCGGCAGCCTGAGCCTGCCCCTGCTGGCCG
GCCTGGTGGCCGCCGACGCCGTGGCCAGCCTGCTGATCGTGGGCGCCGTGTTCCTGTGCGCCAGGCCCAGGCGGAGC
CCtGCCCAGGAGGACGGCAAGGTGTACATCAACATGCCCGGCCGGGGCAGGCGGAAAAGGTCTGGGAGTGGGGCTAC
CAATTTCTCTCTCCTCAAGCAAGCCGGAGACGTTGAGGAAAACCCTGGaCCCATGAGAGTGAAGTTCAGCAGGAGCG
CAGACGCCCCCGCGTACCAGCAGGGCCAGAACCAGCTCTATAACGAGCTCAATCTAGGACGAAGAGAGGAGTACGAT
GTTTTGGACAAGAGACGTGGCCGGGACCCTGAGATGGGGGGAAAGCCGAGAAGGAAGAACCCTCAGGAAGGCCTGTA
CAATGAACTGCAGAAAGATAAGATGGCGGAGGCCTACAGTGAGATTGGGATGAAAGGCGAGCGCCGGAGGGGCAAGG
GGCACGATGGCCTTTACCAGGGTCTCAGTACAGCCACCAAGgacacctacgatgcaTTGCACATGCAGGCCCTGCCC
CCTCGCATGGGCTGGATCCGCGGCCGCAGGAGCCGGCACAGCTGGGAGATGAGCGAGTTCCACAACTACAACCTGGA
CCTGAAGAAGAGCGACTTCAGCACCCGGTGGCAGAAGCAGCGGTGCCCCGTGGTGAAGAGCAAGTGCCGGGAGAACG
CCAGCCCCTTCTTCTTCTGCTGCTTCATCGCCGTGGCtATGGGCATCCGGTTTATAATCATGGTGGCCATCTGGAGC
GCCGTGTTCCTGAACAGCCTGTTCAACCAGGAGGTGCAGATCCCCCTGACCGAGAGCTACTGCGGCCCCTGCCCCAA
GAACTGGATCTGCTACAAGAACAACTGCTACCAGTTCTTCGACGAGAGCAAGAACTGGTACGAGAGCCAGGCCAGCT
GCATGAGCCAGAACGCCAGCCTGCTGAAGGTGTACAGCAAGGAGGACCAGGACCTGCTGAAGCTGGTGAAGAGCTAC
CACTGGATGGGCCTGGTGCACATCCCCACCAACGGCAGCTGGCAGTGGGAGGACGGCAGCATCCTGAGCCCCAACCT
GCTGACCATCATCGAGATGCAGAAGGGCGACTGCGCCCTGTACGCCAGCAGCTTCAAGGGCTACATCGAGAACTGCA
GCACCCCCAACACCTACATCTGCATGCAGCGGACCGTGtaa
SEQ ID NO: 95 (human extracellular NKG2D domain)
LFNQEVQIPLTESYCGPCPKNWICYKNNCYQFFDESKNWYESQASCMSQNASLLKVYSKEDQDLLKLVKSYHWMGLV
HIPTNGSWQWEDGSILSPNLLTIIEMQKGDCALYASSFKGYIENCSTPNTYICMQRTV
SEQ ID NO: 96 (human extracellular NKG2D domain)
IWSAVFLNSLFNQEVQIPLTESYCGPCPKNWICYKNNCYQFFDESKNWYESQASCMSQNASLLKVYSKEDQDLLKLV
KSYHWMGLVHIPTNGSWQWEDGSILSPNLLTIIEMQKGDCALYASSFKGYIENCSTPNTYICMQRTV
SEQ ID NO: 97 (SEQ ID NO: 95 minus 8 most N-terminal amino acids)
PLTESYCGPCPKNWICYKNNCYQFFDESKNWYESQASCMSQNASLLKVYSKEDQDLLKLVKSYHWMGLVHIPTNGSW
QWEDGSILSPNLLTIIEMQKGDCALYASSFKGYIENCSTPNTYICMQRTV
SEQ ID NO: 98 (mouse NKG2D TM domain; UniProt accession no: 054709)
VVRVLAIALAIRFTLNTLMWLAI
SEQ ID NO: 99 (mouse NKG2D TM domain)
KISPMFVVRVLAIALAIRFTLNTLMWLAIFKETFQPV
SEQ ID NO: 100 (rat NKG2D)
MSKCHNYDLKPAKWDTSQEHQKQRSALPTSRPGENGIIRRRSSIEELKISPLFVVRVLVA
AMTIRFTVITLTWLAVFITLLCNKEVSVSSREGYCGPCPNDWICHRNNCYQFFNENKAWN
QSQASCLSQNSSLLKIYSKEEQDFLKLVKSYHWMGLVQSPANGSWQWEDGSSLSPNELTL
VKTPSGTCAVYGSSFKAYTEDCSNPNTYICMKRAV
SEQ ID NO: 101 (rat NKG2D TM domain; UniProt accession no: 070215 aa 52-74)
LFVVRVLVAAMTIRFTVITLTWL
SEQ ID NO: 102 (N5 polypeptide)
MALPVTALLLPLALLLHAARPDYKDDDDKLRPVQAQAQSDCSCSTVSPGVLAGIVMGDLVLTVLIALAVYFLGRLVP
RGRGAAEAATRKQRITETESPYQELQGQRSDVYSDLNTQRPYYKRRKRSGSGEGRGSLLTCGDVEENPGPMIHLGHI
LFLLLLPVAAAQTTPGERSSLPAFYPGTSGSCSGCGSLSLPLLAGLVAADAVASLLIVGAVELCARPRRSPAQEDGK
VYINMPGRGRRKRSGSGATNFSLLKQAGDVEENPGPMKISPMFVVRVLAIALAIRFTLNTLMWLAIFKETFQPVLEN
QEVQIPLTESYCGPCPKNWICYKNNCYQFFDESKNWYESQASCMSQNASLLKVYSKEDQDLLKLVKSYHWMGLVHIP
TNGSWQWEDGSILSPNLLTIIEMQKGDCALYASSFKGYIENCSTPNTYICMQRTV
SEQ ID NO: 103 (nucleic acid encoding polypeptide of SEQ ID NO: 102)
ATGGCTCTGCCTGTGACAGCTCTGCTGCTGCCTCTGGCTCTGCTGCTGCACGCCGCTAGACCCGATTATAAGGACGA
CGACGACAAGCTGAGACCCGTGCAGGCCCAGGCCCAGAGCGACTGCAGCTGCAGCACCGTGAGCCCCGGCGTGCTGG
CCGGCATCGTGATGGGCGACCTGGTGCTGACCGTGCTCATCGCCCTTGCCGTGTACTTCCTGGGCAGACTGGTCCCC
AGGGGCAGAGGAGCTGCCGAGGCCGCTACCAGAAAGCAGAGGATCACCGAGACAGAGAGCCCCTACCAGGAGCTGCA
GGGCCAGAGATCCGACGTGTACAGCGACCTCAACACCCAGAGACCCTATTACAAGAGGCGGAAGCGCTCCGGCTCCG
GCGAGGGCCGCGGCAGCCTGCTGACCTGCGGCGACGTGGAAGAGAACCCCGGACCCATGATCCACCTGGGCCACATC
CTGTTCCTGCTGCTGCTGCCCGTGGCCGCTGCCCAAACAACACCCGGCGAGAGATCCTCCTTGCCCGCTTTCTATCC
CGGAACATCCGGAAGCTGTTCCGGATGTGGATCCCTTTCTTTGCCTTTGCTTGCTGGATTGGTCGCAGCTGACGCTG
TCGCTTCCCTCCTTATTGTCGGAGCTGTCTTCCTGTGCGCCAGGCCCAGGCGGAGCCCTGCCCAGGAGGACGGCAAG
GTGTACATCAACATGCCCGGCCGGGGCAGGCGGAAGCGCTCCGGGAGTGGGGCTACCAATTTCTCTCTCCTCAAGCA
AGCCGGAGACGTTGAGGAAAACCCTGGACCCATGAAAATATCTCCAATGTTCGTTGTTCGAGTCCTTGCTATAGCCT
TGGCAATTCGATTCACCCTTAACACATTGATGTGGCTTGCCATTTTCAAAGAGACGTTTCAGCCAGTACTGTTCAAC
CAGGAGGTGCAGATCCCCCTGACCGAGAGCTACTGCGGCCCCTGCCCAAAAAATTGGATCTGCTACAAGAACAACTG
CTACCAGTTCTTCGACGAGAGCAAGAACTGGTACGAGAGCCAGGCCAGCTGCATGAGCCAGAACGCCAGCCTGCTGA
AGGTGTACAGCAAGGAGGACCAGGACCTGCTGAAGCTGGTGAAGAGCTACCACTGGATGGGCCTGGTGCACATCCCC
ACCAACGGCAGCTGGCAGTGGGAGGACGGCAGCATCCTGAGCCCCAACCTGCTGACCATCATCGAGATGCAGAAGGG
CGACTGCGCCCTGTACGCCAGCAGCTTCAAGGGCTACATCGAGAACTGCAGCACCCCCAACACCTACATCTGCATGC
AGCGGACCGTG
SEQ ID NO: 104 (human IgG1 hinge-aa 218-232 of UniProt: P0DOX5)
EPKSCDKTHTCPPCP
SEQ ID NO: 105 (amino acid sequence of CYAD-01_10 (NKG2D-CD37 + ribosomal skip
peptide + DAP10))
MIHLGHILFLLLLPVAAAQTTPGERSSLPAFYPGTSGSCSGCGSLSLPLLAGLVAADAVASLLIVGAVELCARPRRS
PAQEDGKVYINMPGRGRRKRSGSGATNFSLLKQAGDVEENPGPMRVKFSRSADAPAYQQGQNQLYNELNLGRREEYD
VLDKRRGRDPEMGGKPRRKNPQEGLYNELQKDKMAEAYSEIGMKGERRRGKGHDGLYQGLSTATKDTYDALHMQALP
PRMGWIRGRRSRHSWEMSEFHNYNLDLKKSDFSTRWQKQRCPVVKSKCRENASPFFFCCFIAVAMGIRFIIMVAIWS
AVFLNSLFNQEVQIPLTESYCGPCPKNWICYKNNCYQFFDESKNWYESQASCMSQNASLLKVYSKEDQDLLKLVKSY
HWMGLVHIPTNGSWQWEDGSILSPNLLTIIEMQKGDCALYASSFKGYIENCSTPNTYICMQRTV

1. A method of making an immunoresponsive cell, wherein the method comprises:
(a) genetically modifying an immune cell to express an NKG2D polypeptide; wherein the immune cell has been activated with anti-CD3 and anti-CD28 antibodies, or fragments thereof, conjugated to a nanomatrix; or
(b) activating an immune cell, wherein the immune cell has been genetically modified to express an NKG2D polypeptide and wherein activation is with anti-CD3 and anti-CD28 antibodies, or fragments thereof, conjugated to a nanomatrix.

2. The method of claim 1, wherein the method comprises:
(i) activating the immune cell with anti-CD3 and anti-CD28 antibodies, or fragments thereof, conjugated to a nanomatrix; and
(ii) genetically modifying the immune cell to express the NKG2D polypeptide.

3. The method of claim 1 or claim 2, wherein the immune cell is genetically modified to further express a DAP10/DAP12 fusion polypeptide.

4. The method of claim 3, wherein the fusion polypeptide has the formula, from N-terminus to C-terminus:
A-B-C-D-E,
wherein
A is an optional N-terminal sequence;
B is a DAP10 polypeptide;
C is an optional linker sequence;
D is a DAP12 polypeptide; and
E is an optional C-terminal sequence.

5. The method of claim 3 or claim 4, wherein the DAP10 polypeptide is a functional variant of DAP10 comprising an amino acid sequence having at least about 80%, at least about 85%, at least about 90%, at least about 95%, at least about 97%, at least about 98%, or at least about 99% sequence identity to the DAP10 polypeptide of SEQ ID NO: 1.

6. The method of any one of claims 3 to 5, wherein the DAP10 polypeptide is a functional variant of a DAP10 polypeptide which is a truncated version of the polypeptide having the amino acid sequence of SEQ ID NO: 1.

7. The method of claim 6, wherein the truncated version of DAP10 comprises or consists of amino acids 19-93, 19-69, 1-71, 19-71, 19-48, 49-69, 49-93, or 70-93 of SEQ ID NO: 1.

8. The method of any one of claims 3 to 7, wherein the DAP10 polypeptide comprises or consists of any one of SEQ ID NOs: 1-8.

9. The method of any one of claims 3 to 8, wherein the DAP12 polypeptide is a functional variant of DAP12 comprising an amino acid sequence having at least about 80%, at least about 85%, at least about 90%, at least about 95%, at least about 97%, at least about 98%, or at least about 99% sequence identity to the DAP12 polypeptide of SEQ ID NO: 9.

10. The method of any one of claims 3 to 9, wherein the DAP12 polypeptide is a functional variant of a DAP12 polypeptide which is a truncated version of the polypeptide having the amino acid sequence of SEQ ID NO: 9.

11. The method of claim 10, wherein the truncated version of DAP12 comprises or consists of amino acids 22-113, 62-113, 22-61, or 41-61 of SEQ ID NO: 9.

12. The method of any one of claims 3 to 11, wherein the DAP12 polypeptide comprises or consists of any one of SEQ ID NOs: 9-13.

13. The method of any one of claims 3 to 12, wherein the DAP10 polypeptide and the DAP12 polypeptide are joined by a linker.

14. The method of claim 13, wherein the linker comprises the amino acid sequence recited in any of SEQ ID NOs: 18-46.

15. The method of any one of claims 3 to 14, wherein the fusion polypeptide comprises the sequence of any one of SEQ ID NOs: 60 to 63.

16. The method of any one of the preceding claims, wherein the NKG2D polypeptide is a mammalian polypeptide, optionally a human polypeptide.

17. The method of any one of the preceding claims, wherein the amino acid sequence of the NKG2D polypeptide has at least about 85% sequence identity to the sequence of SEQ ID NO: 14.

18. The method of any one of the preceding claims, wherein the NKG2D polypeptide is a functional variant of the polypeptide having the amino acid sequence of SEQ ID NO: 14.

19. The method of claim 18, wherein the functional variant is a chimeric NKG2D polypeptide.

20. The method of any one of the preceding claims, wherein the immune cell is genetically modified to express a CXCR2 polypeptide.

21. The method of claim 20, wherein the CXCR2 polypeptide is a mammalian polypeptide, optionally a human polypeptide.

22. The method of claim 20 or claim 21, wherein the amino acid sequence of the CXCR2 polypeptide has at least about 85% sequence identity to the sequence of SEQ ID NO: 87.

23. The immunoresponsive cell of claim 22, wherein the CXCR2 polypeptide has the amino acid sequence of SEQ ID NO: 87.

24. The method of any one of the preceding claims, wherein the nanomatrix comprises a matrix of mobile polymer chains conjugated to the anti-CD3 and anti-CD28 antibodies or fragments thereof.

25. The method of claim 24, wherein the polymer chains comprise polysaccharides.

26. The method of any one of the preceding claims, wherein the nanomatrix has a diameter of from about 1 to about 500 nm.

27. The method of any one of the preceding claims, wherein the nanomatrix has a diameter of from about 50 nm to about 200 nm.

28. The method of any one of the preceding claims, wherein the method does not comprise phorbol 12-myristate 13-acetate (PMA), phytohaemagglutinin (PHA) or beads.

29. The method of any one of the preceding claims, wherein the immune cell is a T-cell or a Natural Killer (NK) cell.

30. The method of any one of the preceding claims, wherein the immune cell is an αβ or a γδ T-cell.

31. The method of any one of the preceding claims, wherein the immune cell is a CD4⁺ T-cell.

32. A method of making an immunoresponsive cell, wherein the method comprises:
(a) activating an immune cell, wherein activating does not comprise phorbol 12-myristate 13-acetate (PMA), phytohaemagglutinin (PHA) or beads; and
(b) genetically modifying the immune cell to express the NKG2D polypeptide.

33. An immunoresponsive cell obtainable by the method of any one of claims 1 to 32.

34. An immunoresponsive cell genetically modified to express an NKG2D polypeptide; wherein the immunoresponsive cell has been activated with anti-CD3 and anti-CD28 antibodies, or fragments thereof, conjugated to a nanomatrix.

35. The immunoresponsive cell of claim 34, wherein the immunoresponsive cell is genetically modified to express the NKG2D polypeptide and a fusion polypeptide comprising (i) a DNAX-activating 10 (DAP10) polypeptide, or a functional variant thereof and (ii) a DNAX-activating protein 12 (DAP12) polypeptide, or a functional variant thereof.

36. The immunoresponsive cell of claim 34 or claim 35, wherein the immunoresponsive cell is genetically modified to further express a CXCR2 polypeptide.

37. The immunoresponsive cell of any one of claims 34 to 36, wherein the immunoresponsive cell is a T-cell or a Natural Killer (NK) cell.

38. The immunoresponsive cell of claim 37, wherein the immunoresponsive cell comprises a population of T-cells.

39. The immunoresponsive cell of claim 38, wherein the population of T-cells comprises a CD4:CD8 ratio of at least about 1:1.

40. The immunoresponsive cell of claim 38 or claim 39, wherein the population of T-cells comprises a CD4:CD8 ratio of at least about 2:1.

41. The immunoresponsive cell of any one of claims 37 to 40, wherein the population of T-cells has an expansion rate of at least about five-fold every ten days.

42. A pharmaceutical composition comprising the immunoresponsive cell of any one of claims 33 to 41 and a pharmaceutically or physiologically acceptable diluent and/or carrier.

43. A kit comprising the immunoresponsive cell of any one of claims 33 to 41 or the pharmaceutical composition of claim 42.

44. The immunoresponsive cell of any one of claims 33 to 41 or the pharmaceutical composition of claim 42 for use in the treatment or prevention of a disease, optionally wherein the disease is cancer.

45. Use of the immunoresponsive cell of any one of claims 33 to 41 or the pharmaceutical composition of claim 42 for (i) therapy or (ii) the treatment of cancer.

46. A method of treating or preventing cancer in a subject, wherein the method comprises administering to the subject the immunoresponsive cell of any one of claims 33 to 41 or the pharmaceutical composition of claim 42.

47. The immunoresponsive cell or pharmaceutical composition for use of claim 44, use of claim 45 or method of claim 46, wherein the cancer is breast cancer.

48. The immunoresponsive cell or pharmaceutical composition for use, use, or method of claim 47, wherein the breast cancer is triple-negative breast cancer.
